(19) United States
(12) Patent Application Publication
(10) Pub. No.: US 2025/0250590 A1
(43) Pub. Date: Aug. 7, 2025

(54) CRISPR-CAS13 SYSTEM AND USE THEREOF

(71) Applicants: GUANGZHOU REFORGENE MEDICINE CO., LTD., Guangzhou, Guangdong (CN); ZHEJIANG SYNSORBIO TECHNOLOGY CO., LTD, Shaoxing, Zhejiang (CN)

(72) Inventors: Junbin Liang, Guangzhou, Guangdong (CN); Xingxiang Liang, Guangzhou, Guangdong (CN); Yang Sun, Shaoxing, Zhejiang (CN); Hui Xu, Guangzhou, Guangdong (CN); Kaiwei Si, Guangzhou, Guangdong (CN); Qiuting Li, Guangzhou, Guangdong (CN); Zhiqin Peng, Guangzhou, Guangdong (CN); Desheng Huangfu, Guangzhou, Guangdong (CN)

(21) Appl. No.: 19/194,365

(22) Filed: Apr. 30, 2025

Related U.S. Application Data
(63) Continuation of application No. 18/755,750, filed on Jun. 27, 2024, now Pat. No. 12,297,450.
Application No. 18/755,750 is a continuation of application No. PCT/CN2023/115093, filed on Aug. 25, 2023.

(30) Foreign Application Priority Data
Aug. 26, 2022 (CN) 202211035342.8
Apr. 24, 2023 (CN) 202310457880.4

(51) Int. Cl. C12N 15/90; C12N 9/22; C12N 15/11

(52) U.S. Cl. CPC C12N 15/907; C12N 9/22; C12N 15/11; C12N 2310/20; C12N 2310/531

(57) ABSTRACT

The present invention relates to a CRISPR-Cas13 system and use thereof, and also relates to a Cas13 protein, a fusion protein, and a guide polynucleotide. The Cas13 protein has at least 90% sequence identity compared to SEQ ID NO: 1. The fusion protein comprises the Cas13 protein fused to a protein domain and/or a polypeptide tag. The guide polynucleotide comprises a same-direction repetition sequence and a guide sequence that has been engineered to hybridize with the target RNA. The same-direction repetition sequence has at least 70% sequence identity to any of SEQ ID NOs: 3 and 80-87. The CRISPR-Cas13 system comprises the Cas13 protein that has at least 90% sequence identity to SEQ ID NO: 1, or a coding nucleic acid therefor, and the guide polynucleotide or a coding nucleic acid therefor.

### CROSS-REFERENCE TO RELATED APPLICATIONS

This application is a continuation application of application Ser. No. 18/755,750, filed on Jun. 27, 2024, which is a continuation of PCT/CN2023/115093, filed on Aug. 25, 2023, which claims priorities from Chinese patent application CN202211035342.8, filed on Aug. 26, 2022 and Chinese patent application CN202310457880.4, filed on Apr. 24, 2023, the entire contents of which are hereby incorporated by reference.

### REFERENCE TO SEQUENCE LISTING

The instant application contains a Sequence Listing as an XML file entitled “P25412759USD-SEQ.xml” created on Apr. 29, 2025 and having a size of 447,353 bytes.

### TECHNICAL FIELD

The present disclosure relates to the field of CRISPR gene editing, particularly relates to a CRISPR-Cas13 system and use thereof.

### BACKGROUND

CRISPR-Cas13 is an RNA targeting and editing system based on the bacterial immune system that protects bacteria from viruses. The CRISPR-Cas13 system is similar to the CRISPR-Cas9 system, but unlike Cas9 proteins which target DNA, Cas13 proteins target RNA.

CRISPR-Cas13 belongs to Type VI CRISPR-Cas13 system, which contains a single effector protein, Cas13. Currently, CRISPR-Cas13 can be divided into multiple subtypes (e.g., Cas13a, Cas13b, Cas13c, and Cas13d) according to phylogeny. However, a need remains for a novel Cas13 system that has compact size (e.g., suitable for AAV delivery), high editing efficiency in mammalian cells (e.g., RNA targeting/cleavage activity), and/or low cytotoxicity (e.g., cellular dormancy and apoptosis caused by collateral RNA degradation).

### SUMMARY

One aspect of the disclosure provided herein relates to a Cas13 protein, wherein the amino acid sequence of the Cas 13 protein has at least 90% sequence identity compared to SEQ ID NO: 1.

In some embodiments, the Cas13 protein forms a CRISPR complex with a guide polynucleotide comprising a direct repeat sequence linked to a guide sequence that is engineered to hybridize with a target RNA.

In some embodiments, the Cas13 protein can form a CRISPR complex with a guide polynucleotide, and the CRISPR complex can sequence-specifically bind to the target RNA.

In some embodiments, the Cas13 protein can form a CRISPR complex with a guide polynucleotide comprising a direct repeat sequence linked to a guide sequence that is engineered to guide the sequence-specific binding of the CRISPR complex to the target RNA.

In some embodiments, the amino acid sequence of the Cas13 protein has at least 95% sequence identity compared to SEQ ID NO: 1. In some embodiments, the amino acid sequence of the Cas13 protein has at least 96% sequence identity compared to SEQ ID NO: 1. In some embodiments, the amino acid sequence of the Cas13 protein has at least 97% sequence identity compared to SEQ ID NO: 1. In some embodiments, the amino acid sequence of the Cas13 protein has at least 98% sequence identity compared to SEQ ID NO: 1. In some embodiments, the amino acid sequence of the Cas13 protein has at least 99% sequence identity compared to SEQ ID NO: 1. In some embodiments, the amino acid sequence of the Cas13 protein has at least 99.5% sequence identity compared to SEQ ID NO: 1. In some embodiments, the amino acid sequence of the Cas13 protein is as shown in SEQ ID NO: 1.

The Cas13 protein (i.e. C13-2 protein) with the sequence of SEQ ID NO: 1 in the present disclosure was identified based on bioinformatics analysis of prokaryotic genomes and metagenomes in the CNGB database (China National Gene Bank) and, followed by subsequent activity validation. In some embodiments, the Cas13 protein in the present disclosure derives from the species comprising a genome having an average nucleotide identity (ANI) of ≥95% with the genome of No. CNA0009596 in the CNGB database.

Average nucleotide identity (ANI) is an indicator that evaluates the similarity of all orthologous protein coding genes between two genomes at the nucleic acid level. For bacteria/archaea, the threshold ANI=95% is generally used as the basis for determining whether they are the same species (Richter M, Rosselló-Móra R. Shifting the genetic gold standard for the prokaryotic species definition. Proc Natl Acad Sci USA. 2009 Nov. 10; 106 (45): 19126-31). Therefore, the present disclosure is defined based on the above threshold, and the species with an ANI value≥95% of the reference genome are considered the same species, wherein the Cas13 protein has homology and similar function with the protein claimed by the present disclosure, which belongs to the scope of the present disclosure. ANI analysis tools include programs such as FastANI, JSpecies, etc.

In some embodiments, compared to SEQ ID NO: 1, the amino acid sequence of the Cas13 protein comprises one, two, three, four, five, six, seven or more mutations, such as a single amino acid insertion, a single amino acid deletion, a single amino acid substitution, or a combination thereof.

In some embodiments, the Cas13 protein comprises one or more mutations in the catalytic domain and has reduced RNA cleavage activity.

In some embodiments, the Cas13 protein comprises one mutation in the catalytic domain and has reduced RNA cleavage activity. In some embodiments, the Cas13 protein comprises one or more mutations in one or both HEPN domains and substantially lacks RNA cleavage activity. In some embodiments, the Cas13 protein comprises one mutation in any one of both HEPN domain and substantially lacks RNA cleavage activity.

The expression “substantially lack RNA cleavage activity” refers to the retention of ≤ 50%, ≤40%, ≤30%, ≤20%, ≤10%, ≤5%, or <1% of RNA cleavage activity compared to the wild-type Cas13 protein, or the absence of detectable RNA cleavage activity.

In some embodiments, the RxxxxH motif of the Cas13 protein (x represents any amino acid, RxxxxH can also be referred to as Rx4H or R4xH) comprises one or more mutations and substantially lacks RNA cleavage activity.

In some embodiments, the Cas13 protein comprises a mutation at the position corresponding to the RxxxxH motifs at positions 210-215, 750-755, and/or 785-790 of the reference protein as shown in SEQ ID NO: 1.

In some embodiments, the Cas13 protein comprises a mutation at the position corresponding to the RxxxxH motif at positions 210-215 of the reference protein as shown in SEQ ID NO: 1. In some embodiments, the Cas13 protein comprises a mutation at the position corresponding to the RxxxxH motif at positions 750-755 of the reference protein as shown in SEQ ID NO: 1. In some embodiments, the Cas13 protein comprises a mutation at the position corresponding to the RxxxxH motif at positions 785-790 of the reference protein as shown in SEQ ID NO: 1.

In some embodiments, the Cas13 protein comprises a mutation at the position corresponding to the RxxxxH motifs at positions 210-215 and 750-755 of the reference protein as shown in SEQ ID NO: 1.

In some embodiments, the Cas13 protein comprises a mutation at the position corresponding to the RxxxxH motifs at positions 210-215 and 785-790 of the reference protein as shown in SEQ ID NO: 1.

In some embodiments, the Cas13 protein comprises a mutation at the position corresponding to the RxxxxH motifs at positions 750-755 and 785-790 of the reference protein as shown in SEQ ID NO: 1.

In some embodiments, the Cas13 protein comprises a mutation at the position corresponding to the RxxxxH motifs at positions 210-215, 750-755 and 785-790 of the reference protein as shown in SEQ ID NO: 1.

In some embodiments, the RxxxxH motif is mutated to AxxxxxH, RxxxxA, or AxxxxxA. In some embodiments, the RxxxxH motif is mutated to AxxxxxH. In some embodiments, the RxxxxH motif is mutated to RxxxxA. In some embodiments, the RxxxxH motif is mutated to AxxxxxA.

In some embodiments, the Cas13 protein comprises 1, 2, 3, 4, 5, or 6 mutations at the position corresponding to the amino acid residues R210, H215, R750, H755, R785, and/or H790 of the reference protein as shown in SEQ ID NO: 1. In some embodiments, the amino acid residues of the Cas13 protein are mutated to A (alanine) at the position corresponding to the amino acid residues R210, H215, R750, H755, R785, and/or H790 of the reference protein as shown in SEQ ID NO: 1.

In some embodiments, the amino acid sequence of the Cas13 protein comprises a mutation at the position corresponding to the amino acid residues R210 and H215 of the reference protein as shown in SEQ ID NO: 1. In some embodiments, the amino acid sequence of the Cas13 protein comprises a mutation at the position corresponding to the amino acid residues R750 and H755 of the reference protein as shown in SEQ ID NO: 1. In some embodiments, the amino acid sequence of the Cas13 protein comprises a mutation at the position corresponding to the amino acid residues R785 and H790 of the reference protein as shown in SEQ ID NO: 1.

In some embodiments, the amino acid sequence of the Cas13 protein comprises a mutation at the position corresponding to the amino acid residues R210, H215, R750 and H755 of the reference protein as shown in SEQ ID NO: 1.

In some embodiments, the Cas13 protein comprises a mutation at the position corresponding to the amino acid residues R750, H755, R785 and H790 of the reference protein as shown in SEQ ID NO: 1.

In some embodiments, the Cas13 protein comprises a mutation at the position corresponding to the amino acid residues R210, H215, R785 and H790 of the reference protein as shown in SEQ ID NO: 1.

In some embodiments, the Cas13 protein comprises a mutation at the position corresponding to the amino acid residues R210, H215, R750, H755, R785 and H790 of the reference protein as shown in SEQ ID NO: 1.

In some embodiments, the position corresponding to the amino acid residues R210, R750, or R785 is mutated to A. In some embodiments, the position corresponding to the amino acid residues H215, H755, or H790 is mutated to A. In some embodiments, the position corresponding to the amino acid residues R210, H215, R750, H755, R785, and H790 are all mutated to A.

In some embodiments, the Cas13 protein is obtained by introducing a mutation in the RxxxxH motifs at positions 210-215, 750-755, and/or 785-790 of the sequence as shown in SEQ ID NO: 1.

In some embodiments, the Cas13 protein is obtained by introducing 1, 2, 3, 4, 5, or 6 mutations at positions R210, H215, R750, H755, R785, and/or H790 of the sequence as shown in SEQ ID NO: 1. In some embodiments, the Cas13 protein is obtained by mutating the amino acid residues to A (alanine) at positions R210, H215, R750, H755, R785, and/or H790 of the sequence as shown in SEQ ID NO: 1.

In some embodiments, the Cas13 protein is obtained by mutating the amino acid residues to A at positions R210, H215, R785, and H790 of the sequence as shown in SEQ ID NO: 1. In some embodiments, the Cas13 protein is obtained by mutating the amino acid residues to A at positions R210, H215, R750, and H755 of the sequence as shown in SEQ ID NO: 1. In some embodiments, the Cas13 protein is obtained by mutating the amino acid residues to A at positions R750, H755, R785, and H790 of the sequence as shown in SEQ ID NO: 1. In some embodiments, the Cas13 protein is obtained by mutating the amino acid residues to A at positions R210, H215, R750, H755, R785, and H790 of the sequence as shown in SEQ ID NO: 1.

In some embodiments, the Cas13 protein comprises at least one mutation at the position corresponding to the amino acid residue at positions 40-91, 146-153, 158-176, 182-209, 216-253, 271-287, 341-353, 379-424, 456-477, 521-557, 575-588, 609-625, 700-721, 724-783, 796-815, 828-852 or 880-893 of the reference protein as shown in SEQ ID NO: 1.

In some embodiments, compared with the reference protein as shown in SEQ ID NO: 1, the Cas13 protein comprises any one or more mutations at the position corresponding to the following amino acid residues of the reference protein as shown in SEQ ID NO: 1: R11, N34, R35, R47, R58, R63, R64, N68, N87, N265, N274, R276, R290, R294, N299, N303, R308,
R314, R320, R328, N332, R341, N346, R358, N372, N383, N390, N394, R47+R290, R47+R314, R290+R314, R47+R290+R314, R308+N68, N394+N68, N87+N68, R308+N265, N394+N265, N87+N265, R308+N68+N265, N87+N68+N265, T7, A16, S260, A263, M266, N274, F288, M302, N303, L304, V305, 1311, D313, H324, P326, H327, N332, N346, T353, T360, E365, A373, M380, S382, K395, Y396, D402, D411, S418.

In some embodiments, compared with the reference protein as shown in SEQ ID NO: 1, the Cas13 protein comprises any one or more mutations at the position corresponding to the following amino acid residues of the reference protein as shown in SEQ ID NO: 1: R11, N34, R35, R47, R58, R63, R64, N68, N87, N265, N274, R276, R290, R294, N299, N303, R308, R314.

In some embodiments, compared with the reference protein as shown in SEQ ID NO: 1, the Cas13 protein comprises any one or more mutations at the positions corresponding to the following amino acid residues of the reference protein as shown in SEQ ID NO: 1: R47+R290, R47+R314, R290+R314, R47+R290+R314, N394+N265, N87+N265, A263, M266, N274, F288, V305, 1311, D313, H324, T360, E365, A373, M380, D402, D411, S418.

In some embodiments, the Cas13 protein comprises a mutation at the position corresponding to amino acid residue R11 of the reference protein as shown in SEQ ID NO: 1.

In some embodiments, the Cas13 protein comprises a mutation at the position corresponding to amino acid residue N34 of the reference protein as shown in SEQ ID NO: 1.

In some embodiments, the Cas13 protein comprises a mutation at the position corresponding to amino acid residue R35 of the reference protein as shown in SEQ ID NO: 1.

In some embodiments, the Cas13 protein comprises a mutation at the position corresponding to amino acid residue R47 of the reference protein as shown in SEQ ID NO: 1.

In some embodiments, the Cas13 protein comprises a mutation at the position corresponding to amino acid residue R58 of the reference protein as shown in SEQ ID NO: 1.

In some embodiments, the Cas13 protein comprises a mutation at the position corresponding to amino acid residue R63 of the reference protein as shown in SEQ ID NO: 1.

In some embodiments, the Cas13 protein comprises a mutation at the position corresponding to amino acid residue R64 of the reference protein as shown in SEQ ID NO: 1.

In some embodiments, the Cas13 protein comprises a mutation at the position corresponding to amino acid residue N68 of the reference protein as shown in SEQ ID NO: 1.

In some embodiments, the Cas13 protein comprises a mutation at the position corresponding to amino acid residue N87 of the reference protein as shown in SEQ ID NO: 1.

In some embodiments, the Cas13 protein comprises a mutation at the position corresponding to amino acid residue N265 of the reference protein as shown in SEQ ID NO: 1.

In some embodiments, the Cas13 protein comprises a mutation at the position corresponding to amino acid residue N274 of the reference protein as shown in SEQ ID NO: 1.

In some embodiments, the Cas13 protein comprises a mutation at the position corresponding to amino acid residue R276 of the reference protein as shown in SEQ ID NO: 1.

In some embodiments, the Cas13 protein comprises a mutation at the position corresponding to amino acid residue R290 of the reference protein as shown in SEQ ID NO: 1.

In some embodiments, the Cas13 protein comprises a mutation at the position corresponding to amino acid residue R294 of the reference protein as shown in SEQ ID NO: 1.

In some embodiments, the Cas13 protein comprises a mutation at the position corresponding to amino acid residue N299 of the reference protein as shown in SEQ ID NO: 1.

In some embodiments, the Cas13 protein comprises a mutation at the position corresponding to amino acid residue N303 of the reference protein as shown in SEQ ID NO: 1.

In some embodiments, the Cas13 protein comprises a mutation at the position corresponding to amino acid residue R308 of the reference protein as shown in SEQ ID NO: 1.

In some embodiments, the Cas13 protein comprises a mutation at the position corresponding to amino acid residue R314 of the reference protein as shown in SEQ ID NO: 1.

In some embodiments, the Cas13 protein comprises a mutation at the position corresponding to amino acid residue R320 of the reference protein as shown in SEQ ID NO: 1.

In some embodiments, the Cas13 protein comprises a mutation at the position corresponding to amino acid residue R328 of the reference protein as shown in SEQ ID NO: 1.

In some embodiments, the Cas13 protein comprises a mutation at the position corresponding to amino acid residue N332 of the reference protein as shown in SEQ ID NO: 1.

In some embodiments, the Cas13 protein comprises a mutation at the position corresponding to amino acid residue R341 of the reference protein as shown in SEQ ID NO: 1.

In some embodiments, the Cas13 protein comprises a mutation at the position corresponding to amino acid residue N346 of the reference protein as shown in SEQ ID NO: 1.

In some embodiments, the Cas13 protein comprises a mutation at the position corresponding to amino acid residue R358 of the reference protein as shown in SEQ ID NO: 1.

In some embodiments, the Cas13 protein comprises a mutation at the position corresponding to amino acid residue N372 of the reference protein as shown in SEQ ID NO: 1.

In some embodiments, the Cas13 protein comprises a mutation at the position corresponding to amino acid residue N383 of the reference protein as shown in SEQ ID NO: 1.

In some embodiments, the Cas13 protein comprises a mutation at the position corresponding to amino acid residue N390 of the reference protein as shown in SEQ ID NO: 1.

In some embodiments, the Cas13 protein comprises a mutation at the position corresponding to amino acid residue N394 of the reference protein as shown in SEQ ID NO: 1.

In some embodiments, the Cas13 protein comprises mutations at positions corresponding to amino acid residues R47 and R290 of the reference protein as shown in SEQ ID NO: 1.

In some embodiments, the Cas13 protein comprises mutations at positions corresponding to amino acid residues R47 and R314 of the reference protein as shown in SEQ ID NO: 1.

In some embodiments, the Cas13 protein comprises mutations at positions corresponding to amino acid residues R290 and R314 of the reference protein as shown in SEQ ID NO: 1.

In some embodiments, the Cas13 protein comprises mutations at positions corresponding to amino acid residues R47, R290 and R314 of the reference protein as shown in SEQ ID NO: 1.

In some embodiments, the Cas13 protein comprises mutations at positions corresponding to amino acid residues R308 and N68 of the reference protein as shown in SEQ ID NO: 1.

In some embodiments, the Cas13 protein comprises mutations at positions corresponding to amino acid residues N394 and N68 of the reference protein as shown in SEQ ID NO: 1.

In some embodiments, the Cas13 protein comprises mutations at positions corresponding to amino acid residues N87 and N68 of the reference protein as shown in SEQ ID NO: 1.

In some embodiments, the Cas13 protein comprises mutations at positions corresponding to amino acid residues R308 and N265 of the reference protein as shown in SEQ ID NO: 1.

In some embodiments, the Cas13 protein comprises mutations at positions corresponding to amino acid residues N394 and N265 of the reference protein as shown in SEQ ID NO: 1.

In some embodiments, the Cas13 protein comprises mutations at positions corresponding to amino acid residues N87 and N265 of the reference protein as shown in SEQ ID NO: 1.

In some embodiments, the Cas13 protein comprises mutations at positions corresponding to amino acid residues R308, N68 and N265 of the reference protein as shown in SEQ ID NO: 1.

In some embodiments, the Cas13 protein comprises mutations at positions corresponding to amino acid residues N87, N68 and N265 of the reference protein as shown in SEQ ID NO: 1.

In some embodiments, the Cas13 protein comprises a mutation at the position corresponding to amino acid residue T7 of the reference protein as shown in SEQ ID NO: 1.

In some embodiments, the Cas13 protein comprises a mutation at the position corresponding to amino acid residue A16 of the reference protein as shown in SEQ ID NO: 1.

In some embodiments, the Cas13 protein comprises a mutation at the position corresponding to amino acid residue S260 of the reference protein as shown in SEQ ID NO: 1.

In some embodiments, the Cas13 protein comprises a mutation at the position corresponding to amino acid residue A263 of the reference protein as shown in SEQ ID NO: 1.

In some embodiments, the Cas13 protein comprises a mutation at the position corresponding to amino acid residue M266 of the reference protein as shown in SEQ ID NO: 1.

In some embodiments, the Cas13 protein comprises a mutation at the position corresponding to amino acid residue N274 of the reference protein as shown in SEQ ID NO: 1.

In some embodiments, the Cas13 protein comprises a mutation at the position corresponding to amino acid residue F288 of the reference protein as shown in SEQ ID NO: 1.

In some embodiments, the Cas13 protein comprises a mutation at the position corresponding to amino acid residue M302 of the reference protein as shown in SEQ ID NO: 1.

In some embodiments, the Cas13 protein comprises a mutation at the position corresponding to amino acid residue N303 of the reference protein as shown in SEQ ID NO: 1.

In some embodiments, the Cas13 protein comprises a mutation at the position corresponding to amino acid residue L304 of the reference protein as shown in SEQ ID NO: 1.

In some embodiments, the Cas13 protein comprises a mutation at the position corresponding to amino acid residue V305 of the reference protein as shown in SEQ ID NO: 1.

In some embodiments, the Cas13 protein comprises a mutation at the position corresponding to amino acid residue 1311 of the reference protein as shown in SEQ ID NO: 1.

In some embodiments, the Cas13 protein comprises a mutation at the position corresponding to amino acid residue D313 of the reference protein as shown in SEQ ID NO: 1.

In some embodiments, the Cas13 protein comprises a mutation at the position corresponding to amino acid residue H324 of the reference protein as shown in SEQ ID NO: 1.

In some embodiments, the Cas13 protein comprises a mutation at the position corresponding to amino acid residue P326 of the reference protein as shown in SEQ ID NO: 1.

In some embodiments, the Cas13 protein comprises a mutation at the position corresponding to amino acid residue H327 of the reference protein as shown in SEQ ID NO: 1.

In some embodiments, the Cas13 protein comprises a mutation at the position corresponding to amino acid residue N332 of the reference protein as shown in SEQ ID NO: 1.

In some embodiments, the Cas13 protein comprises a mutation at the position corresponding to amino acid residue N346 of the reference protein as shown in SEQ ID NO: 1.

In some embodiments, the Cas13 protein comprises a mutation at the position corresponding to amino acid residue T353 of the reference protein as shown in SEQ ID NO: 1. In some embodiments, the Cas13 protein comprises a mutation at the position corresponding to amino acid residue T360 of the reference protein as shown in SEQ ID NO: 1.

In some embodiments, the Cas13 protein comprises a mutation at the position corresponding to amino acid residue E365 of the reference protein as shown in SEQ ID NO: 1.

In some embodiments, the Cas13 protein comprises a mutation at the position corresponding to amino acid residue A373 of the reference protein as shown in SEQ ID NO: 1.

In some embodiments, the Cas13 protein comprises a mutation at the position corresponding to amino acid residue M380 of the reference protein as shown in SEQ ID NO: 1.

In some embodiments, the Cas13 protein comprises a mutation at the position corresponding to amino acid residue S382 of the reference protein as shown in SEQ ID NO: 1.

In some embodiments, the Cas13 protein comprises a mutation at the position corresponding to amino acid residue K395 of the reference protein as shown in SEQ ID NO: 1.

In some embodiments, the Cas13 protein comprises a mutation at the position corresponding to amino acid residue Y396 of the reference protein as shown in SEQ ID NO: 1.

In some embodiments, the Cas13 protein comprises a mutation at the position corresponding to amino acid residue D402 of the reference protein as shown in SEQ ID NO: 1.

In some embodiments, the Cas13 protein comprises a mutation at the position corresponding to amino acid residue D411 of the reference protein as shown in SEQ ID NO: 1.

In some embodiments, the Cas13 protein comprises a mutation at the position corresponding to amino acid residue S418 of the reference protein as shown in SEQ ID NO: 1.

In some embodiments, compared with the reference protein as shown in SEQ ID NO: 1, the Cas13 protein has the mutation to the same amino acid residue at the position corresponding to the mutation site in Table 24 or in Table 29 of the reference protein as shown in SEQ ID NO: 1.

In some embodiments, compared with the reference protein as shown in SEQ ID NO: 1, the Cas13 protein comprises the same mutations at the position corresponding to the mutation site in Table 24 or in Table 29 of the reference protein as shown in SEQ ID NO: 1.

In some embodiments, the Cas13 protein is obtained by introducing any one or more mutations at the following positions of the sequence as shown in SEQ ID NO: 1: R11, N34, R35, R47, R58, R63, R64, N68, N87, N265, N274, R276, R290, R294, N299, N303, R308, R314, R320, R328, N332, R341, N346, R358, N372, N383, N390, N394, R47+R290, R47+R314, R290+R314, R47+R290+R314, R308+N68, N394+N68, N87+N68, R308+N265, N394+N265, N87+N265, R308+N68+N265, N87+N68+N265, T7, A16, S260, A263, M266, N274, F288, M302, N303, L304, V305, 1311, D313, H324, P326, H327, N332, N346, T353, T360, E365, A373, M380, S382, K395, Y396, D402, D411 and S418.

In some embodiments, the Cas13 protein is obtained by introducing any one or more mutations at the following positions of the sequence as shown in SEQ ID NO: 1: N34, R64, N68, N265, R276, R294, N299, R314, R47+R290, R47+R314, R290+R314, R47+R290+R314, N394+N265, N87+N265, A263, M266, N274, F288, V305, 1311, D313, H324, T360, E365, A373, M380, D402 and D411.

In some embodiments, the Cas13 protein is obtained by introducing any one or more mutations in Table 24 or in Table 29 to the sequence as shown in SEQ ID NO: 1.

In some embodiments, the Cas13 protein is obtained by sequence deletion at the position corresponding to the amino acid residue at the position of 91-120, 141-180, 211-240, 331-360, 351-400, 431-460, 461-500, 511-550, 611-640, 631-660, 661-690, 691-760, 821-860, or 861-890 of the reference protein as shown in SEQ ID NO: 1.

In some embodiments, the Cas13 protein comprises the deletion of one or more amino acids at the position corresponding to the amino acid residue at the position of 348-350, 521-556, or 883-893 of the reference protein as shown in SEQ ID NO: 1.

In some embodiments, ≤300, ≤200, ≤150, ≤100, ≤90, ≤80, ≤70, ≤60, ≤50, ≤40, ≤30, ≤20, or ≤10 amino acid residues are deleted in the sequence deletion.

In some embodiments, the Cas13 protein is obtained by sequence deletion at the position of 91-120, 141-180, 211-240, 331-360, 351-400, 431-460, 461-500, 511-550, 611-640, 631-660, 661-690, 691-760, 821-860, or 861-890 of the sequence as shown in SEQ ID NO: 1.

The sequence deletion is a deletion of one or more amino acid residues.

Another aspect of the disclosure provided herein relates to a fusion protein.

In some embodiments of this disclosure, the fusion protein comprises the Cas13 protein described herein or a functional fragment thereof, and any one or more of the following fused to the Cas13 protein or the functional fragment thereof: a cytosine deaminase domain, an adenosine deaminase domain, a translational activation domain, a translational repression domain, an RNA methylation domain, an RNA demethylation domain, a nuclease domain, a splicing factor domain, a reporter domain, an affinity domain, a subcellular localization signal, a reporter tag, and an affinity tag.

In some embodiments of this disclosure, the fusion protein comprises the Cas13 protein described herein or a functional fragment thereof, and any one or more of the following fused to the Cas13 protein or the functional fragment thereof: a cytosine deaminase domain, an adenosine deaminase domain, a translational activation domain, a translational repression domain, an RNA methylation domain, an RNA demethylation domain, a nuclease domain, a splicing factor domain, a subcellular localization signal, a reporter tag, and an affinity tag.

In some embodiments of this disclosure, the fusion protein comprises the Cas13 protein described herein or the functional fragment thereof fused to a homologous or heterologous protein domain and/or a peptide tag. In some embodiments, the fusion does not alter the original function of the Cas13 protein or the functional fragments thereof.

The expression “do not alter the original function of the Cas13 protein or the functional fragments thereof” means that the fused protein still has the ability to recognize, bind and/or cleave a target RNA when used in combination with a gRNA. The ability of the fused protein to recognize, bind or cleave the target RNA when used in combination with the gRNA may be increased or decreased compared to the ability of the Cas13 protein to recognize, bind or cleave the target RNA when used in combination with the gRNA. As long as the fused protein can effectively recognize, bind or cleave the target RNA when used in combination with the gRNA, it belongs to the situation of “do not alter the original function of the Cas13 protein”.

In some embodiments, the fusion protein comprises the Cas13 protein described herein fused to a homologous or heterologous protein domain and/or a peptide tag. In some embodiments, the fusion protein comprises the functional fragment of the Cas13 protein described herein fused to a homologous or heterologous protein domain and/or a peptide tag. For example, in some embodiments, the functional fragment is a fragment obtained from the Cas13 protein by deleting a partial sequence of the nuclease domain. In some embodiments, the functional fragment is a fragment obtained from the Cas13 protein by deleting the sequences corresponding to HEPN-1_I, HEPN-1_II, HEPN-2, NTD, Helical-1, and/or Helical-2 of C13-2. In some embodiments, the functional fragment is a fragment obtained from the Cas13 protein by deleting the sequences corresponding to HEPN-1_I, HEPN-1_II, and/or HEPN-2 of C13-2. In some embodiments, the functional fragment is a fragment obtained from the Cas13 protein by deleting the sequence corresponding to HEPN-1_I of C13-2. In some embodiments, the functional fragment is a fragment obtained from the Cas13 protein by deleting the sequence corresponding to HEPN-1_II of C13-2. In some embodiments, the functional fragment is a fragment obtained from the Cas13 protein by deleting the sequence corresponding to HEPN-2 of C13-2. In some embodiments, the functional fragment is a fragment obtained from the Cas13 protein by deleting the sequences corresponding to NTD and HEPN-1 I of C13-2.

In some embodiments, the Cas13 protein or the functional fragment thereof is fused to any one or more selected from the following: a cytosine deaminase domain, an adenosine deaminase domain, a translational activation domain, a translational repression domain, an RNA methylation domain, an RNA demethylation domain, a nuclease domain, a splicing factor domain, a reporter domain, an affinity domain, a subcellular localization signal, a reporter tag, and an affinity tag.

In some embodiments, the Cas13 protein or the functional fragment thereof is fused to a subcellular localization signal. In some embodiments, the subcellular localization signal is optionally selected from a nuclear localization signal (NLS), a nuclear export signal (NES), a chloroplast localization signal, or a mitochondrial localization signal.

In some embodiments, the Cas13 protein or the functional fragment thereof is fused to any one or more selected from the following: a cytosine deaminase domain, an adenosine deaminase domain, a translational activation domain, a translational repression domain, an RNA methylation domain, an RNA demethylation domain, a nuclease domain, a splicing factor domain, a reporter domain, an affinity domain, a subcellular localization signal, a reporter tag, and an affinity tag.

In some embodiments, the Cas13 protein or the functional fragment thereof is fused to a subcellular localization signal. In some embodiments, the subcellular localization signal is optionally selected from a nuclear localization signal (NLS), a nuclear export signal (NES), a chloroplast localization signal, or a mitochondrial localization signal.

In some embodiments, the Cas13 protein or the functional fragment thereof is fused to a homologous or heterologous nuclear localization signal (NLS). In some embodiments, the Cas13 protein or the functional fragment thereof is fused to a homologous or heterologous nuclear export signal (NES).

In some embodiments, the Cas13 protein or the functional fragment thereof is covalently linked to a protein domain and/or a peptide tag. In some embodiments, the Cas13 protein or the functional fragment is covalently linked to a protein domain and/or a peptide tag directly. In some embodiments, the Cas13 protein or the functional fragment thereof is covalently linked to a protein domain and/or a peptide tag by a linking sequence; furthermore, in some embodiments, the linking sequence is an amino acid sequence.

In some embodiments, the Cas13 protein or the functional fragment thereof of the fusion protein is linked to a homologous or heterologous protein domain and/or a polypeptide tag by a rigid linking peptide sequence. In some embodiments, the Cas13 protein portion of the fusion protein is linked to a homologous or heterologous protein domain and/or a polypeptide tag by a flexible linking peptide sequence. In some embodiments, the rigid linking peptide sequence is A (EAAAK) 3A (SEQ ID NO: 279). In some embodiments, the flexible linking peptide sequence is (GGGGS) 3 (SEQ ID NO: 280).

In some embodiments, the fusion protein can form a CRISPR complex with a guide polynucleotide, wherein the CRISPR complex can sequence-specifically bind to the target RNA. In some embodiments, the fusion protein comprises the Cas13 protein described herein fused to a homologous or heterologous protein domain and/or a peptide tag; wherein the fusion protein can form a CRISPR complex with a guide polynucleotide, and the CRISPR complex can sequence-specifically bind to the target RNA. In some embodiments, the fusion protein comprises the functional fragment of the Cas13 protein described herein fused to a homologous or heterologous protein domain and/or a peptide tag; wherein the fusion protein can form a CRISPR complex with a guide polynucleotide, and the CRISPR complex can sequence-specifically bind to the target RNA.

In some embodiments, the fusion protein can form a CRISPR complex with a guide polynucleotide comprising a direct repeat sequence linked to a guide sequence that is engineered to guide the sequence-specific binding of the CRISPR complex to the target RNA. In some embodiments, the fusion protein comprises the Cas13 protein described herein fused to a homologous or heterologous protein domain and/or a peptide tag; wherein the fusion protein can form a CRISPR complex with a guide polynucleotide comprising a direct repeat sequence linked to a guide sequence that is engineered to guide the sequence-specific binding of the CRISPR complex to the target RNA. In some embodiments, the fusion protein comprises a functional fragment of the Cas13 protein described herein fused to a homologous or heterologous protein domain and/or a peptide tag; wherein the fusion protein can form a CRISPR complex with a guide polynucleotide comprising a direct repeat sequence linked to a guide sequence that is engineered to guide the sequence-specific binding of the CRISPR complex to the target RNA.

In some embodiments, the fusion protein can form a CRISPR complex with a guide polynucleotide, and the CRISPR complex can sequence-specifically bind to and cleave the target RNA. In some embodiments, the fusion protein comprises the Cas13 protein described herein fused to a homologous or heterologous protein domain and/or a peptide tag; wherein the fusion protein can form a CRISPR complex with a guide polynucleotide, and the CRISPR complex can sequence-specifically bind to the target RNA. In some embodiments, the fusion protein comprises the functional fragment of the Cas13 protein described herein fused to a homologous or heterologous protein domain and/or a peptide tag; wherein the fusion protein can form a CRISPR complex with a guide polynucleotide, and the CRISPR complex can sequence-specifically bind to the target RNA.

In some embodiments, the structure of the fusion protein is NLS-Cas13 protein-SV40 NLS-nucleoplastin NLS.

Another aspect of the disclosure provided herein relates to a guide polynucleotide comprising (i) a direct repeat sequence having at least 50% sequence identity compared to any one of SEQ ID NO: 3 and SEQ ID NOs: 80-87, wherein the direct repeat sequence is linked to (ii) a homologous or heterologous guide sequence engineered to hybridize with a target RNA, wherein the guide polynucleotide can form a CRISPR complex with a Cas13 protein and guide the sequence-specific binding of the CRISPR complex to the target RNA.

In some embodiments, the Cas13 protein is Cas13a, Cas13b, Cas13c, or Cas13d. In some embodiments, the amino acid sequence of the Cas13 protein has at least 90%, at least 95%, at least 98%, or at least 99% sequence identity compared to SEQ ID NO: 1.

In some embodiments, the direct repeat sequence has at least 55%, at least 60%, at least 65%, at least 70%, at least 75%, at least 80%, at least 85%, at least 90%, at least 95%, or 100% sequence identity compared to any one of SEQ ID NO: 3 and SEQ ID NOs: 80-87. In some embodiments, the direct repeat sequence has at least 80% sequence identity compared to any one of SEQ ID NO: 3 and SEQ ID NOs: 80-87. In some embodiments, the direct repeat sequence has at least 85% sequence identity compared to any one of SEQ ID NO: 3 and SEQ ID NOs: 80-87. In some embodiments, the direct repeat sequence has at least 90% sequence identity compared to any one of SEQ ID NO: 3 and SEQ ID NOs: 80-87. In some embodiments, the direct repeat sequence has at least 95% sequence identity compared to any one of SEQ ID NO: 3 and SEQ ID NOs: 80-87. In some embodiments, the direct repeat sequence has 100% sequence identity compared to any one of SEQ ID NO: 3 and SEQ ID NOs: 80-87.

In some embodiments, the direct repeat sequence has at least 50%, at least 55%, at least 60%, at least 65%, at least 70%, at least 75%, at least 80%, at least 85%, at least 90%, at least 95%, or 100% sequence identity compared to any one of SEQ ID NOs: 3, 81, 82, 84, and 87.

In some embodiments, the direct repeat sequence has at least 70%, at least 75%, at least 80%, at least 85%, at least 90%, at least 95%, or 100% sequence identity compared to any one of SEQ ID NOs: 3 to 87.

In some embodiments, the 26th base of the direct repeat sequence corresponding to SEQ ID NO: 3 is A.

In some embodiments, the direct repeat sequence is

(SEQ ID NO: 277)
GGAAGATN₁ACTCTACAAACCTGTAGN₂GN₃N₄N₅N₆N₇N₈N₉N₁₀N₁₁;

wherein N1 and N3-Nu are optionally selected from A, C, G, T; and N2 is optionally selected from A and G.

In some embodiments, the direct repeat sequence is GGAAGATN12ACTCTACAAACCTGTAGN13GN14N15N16N17N18N19N20N21N22 (SEQ ID NO: 278); wherein N12, N13, N19 and N21 are optionally selected from A and G, N14 is optionally selected from A and T, N15 and N16 are optionally selected from C and T, N17 and N18 are optionally selected from G and T, and N20 and N22 are optionally selected from C and G.

In some embodiments, the guide sequence is positioned at the 3′ end of the direct repeat sequence. In some embodiments, the guide sequence is positioned at the 5′ end of the direct repeat sequence.

In some embodiments, the guide sequence comprises 15-35 nucleotides. In some embodiments, the guide sequence hybridizes with the target RNA with no more than one nucleotide mismatch. In some embodiments, the direct repeat sequence comprises 25 to 40 nucleotides.

In some embodiments, the guide polynucleotide further comprises an aptamer sequence. In some embodiments, the aptamer sequence is inserted into a loop of the guide polynucleotide. In some embodiments, the aptamer sequence comprises an MS2 aptamer sequence, a PP7 aptamer sequence, or a QB aptamer sequence.

In some embodiments, the guide polynucleotide comprises a modified nucleotide. In some embodiments, the modified nucleotide comprises 2′-O-methyl, 2′-O-methyl-3′-phosphorothioate, or 2′-O-methyl-3′-thioPACE.

In some embodiments, the target RNA of the guide polynucleotide is located in the nucleus of a eukaryotic cell.

In some embodiments, the target RNA is optionally selected from TTR RNA, SOD1 RNA, PCSK9 RNA, VEGFA RNA, VEGFR1 RNA, PTBP1 RNA, AQp1 RNA, or ANGPTL3 RNA. Optionally, in some embodiments, the target RNA is selected from VEGFARNA, PTBP1 RNA, AQp1 RNA, or ANGPTL3 RNA. Furthermore, the guide sequence is optionally selected from the sequences as shown in SEQ ID NO: 5, SEQ ID NO: 6, and SEQ ID NOs: 42-49 (used for targeting AQp1 RNA, PTBP1 RNA, and ANGPTL3 RNA, respectively). Preferably, the guide sequence is optionally selected from the sequences as shown in SEQ ID NO: 5, SEQ ID NO: 6, SEQ ID NO: 43, and SEQ ID NOs: 45-47.

In some embodiments, the Cas13 protein is Cas13a, Cas13b, Cas13c, or Cas13d. In some embodiments, the Cas13 protein is Cas13d. In some embodiments, the Cas13 protein has at least 90%, at least 95%, at least 98%, or at least 99% sequence identity compared to SEQ ID NO: 1.

Another aspect of the disclosure provided herein relates to a CRISPR-Cas13 system comprising: the Cas13 protein or the fusion protein described herein, or a nucleic acid encoding the Cas13 protein or the fusion protein, and a guide polynucleotide or a nucleic acid encoding the guide polynucleotide; wherein the guide polynucleotide comprises a direct repeat sequence linked to a guide sequence that is engineered to hybridize with the target RNA; and the guide polynucleotide can form a CRISPR complex with the Cas13 protein or the fusion protein and guide the sequence-specific binding of the CRISPR complex to the target RNA.

In some embodiments, the target RNA is optionally selected from TTR RNA, SOD1 RNA, PCSK9 RNA, VEGFA RNA, VEGFR1 RNA, PTBP1 RNA, AQp1 RNA, or ANGPTL3 RNA. Optionally, in some embodiments, the target RNA is optionally selected from VEGFA RNA, PTBP1 RNA, AQp1 RNA, or ANGPTL3 RNA. In some embodiments, the guide sequence is optionally selected from the sequences as shown in SEQ ID NO: 5, SEQ ID NO: 6, and SEQ ID NOs: 42-49 (used for targeting AQp1 RNA, PTBP1 RNA, and ANGPTL3 RNA, respectively). In some embodiments, the guide sequence is optionally selected from the sequences as shown in SEQ ID NO: 5, SEQ ID NO: 6, SEQ ID NO: 43, and SEQ ID NOs: 45-47.

In some embodiments, the direct repeat sequence has at least 70%, at least 75%, at least 80%, at least 85%, at least 90%, at least 95%, or 100% sequence identity compared to any one of SEQ ID NOs: 3, 81, 82, 84, and 87.

In some embodiments, the fusion protein comprises the Cas13 protein described herein or the functional fragment thereof fused to a homologous or heterologous protein domain and/or a peptide tag.

In some embodiments, the portion of the Cas13 protein or the functional fragment thereof of the fusion protein is fused to a homologous or heterologous nuclear localization signal (NLS). In some embodiments, the portion of the Cas13 protein or the functional fragment thereof of the fusion protein is fused to a homologous or heterologous nuclear export signal (NES).

In some embodiments, the Cas13 protein comprises a mutation in a catalytic domain and has reduced RNA cleavage activity. In some embodiments, the Cas13 protein comprises a mutation in one or both HEPN domains and substantially lacks RNA cleavage activity. In some embodiments, the “substantially lack RNA cleavage activity” refers to the retention of ≤50%, ≤40%, ≤30%, ≤20%, ≤10%, ≤5%, or ≤1% of RNA cleavage activity compared to wild-type Cas13 protein, or the absence of detectable RNA cleavage activity.

In some embodiments, the Cas13 protein or the functional fragments thereof of the fusion protein is covalently linked to a homologous or heterologous protein domain and/or a peptide tag directly. In some embodiments, the Cas 13 protein part of the fusion protein is linked to a homologous or heterologous protein domain and/or a peptide tag by a peptide sequence.

In some embodiments, the protein domain comprises a cytosine deaminase domain, an adenosine deaminase domain, a translational activation domain, a translational repression domain, an RNA methylation domain, an RNA demethylation domain, a nuclease domain, or a splicing factor domain. In some embodiments, the Cas13 protein is covalently linked to an affinity tag or a reporter tag.

In some embodiments, the Cas13 protein has at least 95% sequence identity compared to SEQ ID NO: 1. In some embodiments, the Cas13 protein has at least 97% sequence identity compared to SEQ ID NO: 1. In some embodiments, the Cas13 protein has at least 98% sequence identity compared to SEQ ID NO: 1. In some embodiments, the Cas13 protein has at least 99% sequence identity compared to SEQ ID NO: 1. In some embodiments, the Cas13 protein has at least 99.5% sequence identity compared to SEQ ID NO: 1. In some embodiments, the Cas13 protein comprises the sequence as shown in SEQ ID NO: 1.

In some embodiments, the Cas13 protein derives from the species comprising a genome that has an average nucleotide identity (ANI) of ≥95% with the genome no. CNA0009596 in the CNGB database.

In some embodiments, the Cas13 protein does not exhibit a protospacer flanking sequence (PFS) requirement for RNA cleavage.

In some embodiments, the guide sequence is positioned at the 3′ end of the direct repeat sequence. In some embodiments, the guide sequence is positioned at the 5′ end of the direct repeat sequence.

In some embodiments, the guide sequence comprises 15-35 nucleotides. In some embodiments, the guide sequence hybridizes with the target RNA with no more than one nucleotide mismatch.

In some embodiments, the direct repeat sequence comprises 25 to 40 nucleotides.

In some embodiments, the direct repeat sequence has at least 80% sequence identity compared to any one of SEQ ID NO: 3 and SEQ ID NOs: 80-87. In some embodiments, the direct repeat sequence has at least 90% sequence identity compared to any one of SEQ ID NO: 3 and SEQ ID NOs: 80-87. In some embodiments, the direct repeat sequence has at least 95% sequence identity compared to any one of SEQ ID NO: 3 and SEQ ID NOs: 80-87. In some embodiments, the direct repeat sequence has 100% sequence identity compared to any one of SEQ ID NO: 3 and SEQ ID NOs: 80-87. In some embodiments, the direct repeat sequence is optionally selected from SEQ ID NO: 3 and SEQ ID NOs: 80-87.

In some embodiments, the guide polynucleotide further comprises an aptamer sequence. In some embodiments, the aptamer sequence is inserted into a loop of the guide polynucleotide. In some embodiments, the aptamer sequence comprises an MS2 aptamer sequence, a PP7 aptamer sequence, or a QB aptamer sequence.

In some embodiments, the CRISPR-Cas13 system comprises a fusion protein comprising an adaptor protein and a homologous or heterologous protein domain, or a nucleic acid encoding the fusion protein; wherein the adaptor protein is capable of binding to the aptamer sequence.

In some embodiments, the adaptor protein comprises MS2 phage coat protein, PP7 phage coat protein, or QB phage coat protein. In some embodiments, the protein domain comprises a cytosine deaminase domain, an adenosine deaminase domain, a translational activation domain, a translational repression domain, an RNA methylation domain, an RNA demethylation domain, a nuclease domain, a splicing factor domain, a reporter domain, an affinity domain, a reporter tag and an affinity tag.

In some embodiments, the guide polynucleotide comprises a modified nucleotide. In some embodiments, the modified nucleotide comprises 2′-O-methyl, 2′-O-methyl-3′-phosphorothioate, or 2′-O-methyl-3′-thioPACE.

In some embodiments, the Cas13 protein or fusion protein and the guide polynucleotide do not naturally occur together.

Another aspect of the disclosure provided herein relates to a CRISPR-Cas13 system, wherein the CRISPR-Cas13 system comprises any one of the Cas13 protein, the fusion protein thereof, or the nucleic acid encoding the same, and the guide polynucleotides, or the nucleic acid encoding the same described herein.

Another aspect of the disclosure provided herein relates to a vector system comprising the CRISPR-Cas13 system described herein, wherein the vector system comprises one or more vectors comprising a polynucleotide sequence encoding the Cas13 protein or the fusion protein described herein and a polynucleotide sequence encoding the guide polynucleotide.

Another aspect of the disclosure provided herein relates to an adeno-associated viral (AAV) vector comprising the CRISPR-Cas13 system described herein, wherein the AAV vector comprises a DNA sequence encoding the Cas13 protein or the fusion protein described herein and the guide polynucleotide.

Another aspect of the disclosure provided herein relates to a lipid nanoparticle comprising the CRISPR-Cas13 system described herein, wherein the lipid nanoparticle comprises the guide polynucleotide described herein and an mRNA encoding the Cas13 protein or the fusion protein described herein.

Another aspect of the disclosure provided herein relates to a lentiviral vector comprising the CRISPR-Cas13 system described herein, wherein the lentiviral vector comprises the guide polynucleotide described herein and an mRNA encoding the Cas 13 protein or the fusion protein described herein. In some embodiments, the lentiviral vector is pseudotyped with a homologous or heterologous envelope protein such as VSV-G. In some embodiments, the mRNA encoding the Cas13 protein or fusion protein is linked to an aptamer sequence.

Another aspect of the disclosure provided herein relates to a ribonucleoprotein complex comprising the CRISPR-Cas13 system described herein, wherein the ribonucleoprotein complex is formed by the guide polynucleotide described herein and the Cas13 protein or the fusion protein described herein.

Another aspect of the disclosure provided herein relates to a viral-like particle comprising the CRISPR-Cas13 system described herein, wherein the viral-like particle comprises a ribonucleoprotein complex formed by the guide polynucleotide described herein and the Cas13 protein or the fusion protein described herein. In some embodiments, the Cas13 protein or the fusion protein is fused to a gag protein.

Another aspect of the disclosure provided herein relates to a eukaryotic cell comprising the CRISPR-Cas13 system described herein. In some embodiments, the eukaryotic cell is a mammalian cell. In some embodiments, the eukaryotic cell is a human cell.

Another aspect of the disclosure provided herein relates to a pharmaceutical composition comprising the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein.

Another aspect of the disclosure provided herein relates to a pharmaceutical composition comprising the CRISPR-Cas13 system described herein.

Another aspect of the disclosure relates to an in vitro composition comprising the CRISPR-Cas13 system described herein, and a labeled detector RNA that is not hybridizable with or targeted by the guide polynucleotide.

Another aspect of the disclosure provided herein relates to an isolated nucleic acid encoding the Cas13 protein or the fusion protein described herein.

Another aspect of the disclosure provided herein relates to an isolated nucleic acid encoding the guide polynucleotide described herein.

Another aspect of the disclosure provided herein relates to a CRISPR-Cas13 system comprising any one of the Cas13 protein or the nucleic acids encoding the same, and the guide polynucleotides or the nucleic acid encoding the same described herein.

Another aspect of the disclosure provided herein relates to a use of the CRISPR-Cas13 system described herein in the detection of a target RNA in a nucleic acid sample suspected of comprising the target RNA or in the preparation of a reagent for detecting a target RNA in a nucleic acid sample suspected of comprising the target RNA.

Another aspect of the disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in any one of the following or in the preparation of a reagent for achieving any one of the following schemes: cleaving one or more target RNA molecules or nicking one or more target RNA molecules; activating or upregulating one or more target RNA molecules; activating or inhibiting translation of one or more target RNA molecules; inactivating one or more target RNA molecules; visualizing, labeling or detecting one or more target RNA molecules; binding one or more target RNA molecules; transporting one or more target RNA molecules; and masking one or more target RNA molecules.

Another aspect of the disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in the cleavage of one or more target RNA molecules or in the preparation of a reagent for cleaving one or more target RNA molecules.

Another aspect of the disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in binding one or more target RNA molecules.

Another aspect of the disclosure provided herein relates to a use of the CRISPR-Cas13 system described herein in preparing a reagent for binding or cleaving one or more target RNA molecules.

Another aspect of the disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in cleaving or editing a target RNA in a mammalian cell; wherein the editing is base editing.

Another aspect of the disclosure provided herein relates to a use of the CRISPR-Cas13 system described herein in preparing a reagent for cleaving or editing a target RNA in a mammalian cell; wherein the editing is a base editing.

Another aspect of the present disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in activating or upregulating one or more target RNA molecules or in preparing a reagent for activating or upregulating one or more target RNA molecules.

Another aspect of the disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in inhibiting the translation of one or more target RNA molecules or in preparing a reagent for inhibiting the translation of one or more target RNA molecules.

Another aspect of the disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in inactivating one or more target RNA molecules or in preparing a reagent for inactivating one or more target RNA molecules.

Another aspect of the disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in visualizing, labeling or detecting one or more target RNA molecules or in preparing a reagent for visualizing, labeling or detecting one or more target RNA molecules.

Another aspect of the disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in transporting one or more target RNA molecules or in preparing a reagent for transporting one or more target RNA molecules.

Another aspect of the disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in masking one or more target RNA molecules or in preparing a reagent for masking one or more target RNA molecules.

Another aspect of the disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in the diagnosis, treatment, or prevention of a disease or disorder associated with the target RNA.

Another aspect of the disclosure provided herein relates to a use of the CRISPR-Cas13 system described herein in the diagnosis, treatment, or prevention of a disease or disorder associated with the target RNA.

Another aspect of the disclosure provided herein relates to a method for diagnosing, treating, or preventing a disease or disorder associated with the target RNA, wherein the method is to administer the Cas13 protein described herein, the fusion protein described herein, the guide polynucleotide described herein, the CRISPR-Cas13 system described herein, or the isolated nucleic acid described herein to a sample of a subject in need thereof, or to a subject in need thereof.

Another aspect of the disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in the manufacture of a medicament for the diagnosis, treatment, or prevention of a disease or disorder associated with the target RNA.

Another aspect of the disclosure provided herein relates to a use of the CRISPR-Cas13 system described herein in the manufacture of a medicament for the diagnosis, treatment, or prevention of a disease or disorder associated with the target RNA.

Based on common knowledge in the field, the above preferred conditions can be combined arbitrarily to obtain the preferred examples of the present disclosure.

The reagents and raw materials used in the present disclosure are commercially available.

### BRIEF DESCRIPTION OF THE DRAWINGS

FIG. 1 shows the CRISPR locus of a CRISPR-Cas13 system, including a CRISPR array and coding sequences for C13-2 protein.

FIG. 2 shows the structure of the C13-2 guide polynucleotide, which consists of a direct repeat sequence and a guide sequence. In this figure the scaffold sequence is the same as the direct repeat sequence; the guide sequence consists of a variable number of various nucleotides, with N representing any nucleotide in the figure. The secondary structure of the direct repeat sequence (SEQ ID NO: 3) is predicted and obtained by RNAfold, which is shown in the figure. The stem-loop structure could be seen, and the stem region contains various complementary base pairs.

FIG. 3 shows expression and purification of recombinant C13-2 protein.

FIG. 4 shows high activity of C13-2 in downregulating PTBP1 (Polypyrimidine Tract Binding Protein 1) RNA in 293T cells.

FIG. 5 shows higher activity of C13-2 compared to CasRx and shRNA in downregulating AQp1 (Aquaporin 1) RNA in 293T cells.

FIG. 6 shows higher activity of C13-2 compared to C13-113 and C13-114 in downregulating PTBP1 RNA in 293T cells.

FIG. 7 shows downregulation of ANGPTL3 (Angiopoietin-like 3) RNA in 293T cells by C13-2.

FIG. 8 shows in silico predicted C13-2 domain; in the figure, positions 1-95 are NTD domain, positions 96-255 are HEPN-1_I domain, positions 256-417 are Helical-1 domain, positions 418-504 are HEPN-1_II domain, positions 505-651 are Helical-2 domain, and positions 652-893 are HEPN-2 domain.

FIG. 9 shows the editing effect targeting VEGFA RNA when using different DRs.

FIG. 10 shows the editing effect targeting PTBP1 RNA when using different DRs.

FIG. 11 shows the effect comparison of C13-2 with known Cas13 tools targeting VEGFA RNA.

FIG. 12 shows the effect comparison of C13-2 with known Cas13 tools targeting PTBP1 RNA.

FIG. 13 shows the sequencing peak map of dC13-2 after single-base editing; the sequence shown is SEQ ID NO: 284, and the corresponding amino acid sequence is as shown in SEQ ID NO: 283.

FIG. 14 shows the VEGFA RNA levels tested by qPCR after targeting editing of the mutants.

FIG. 15 shows the VEGFA RNA levels tested by RNAseq after editing.

FIG. 16 shows the editing efficiency of the mutants tested by qPCR on AR RNA.

FIG. 17 shows the AR RNA levels tested by RNAseq after editing.

FIG. 18 shows the sequence alignment results of the direct repeat sequences DRrc (SEQ ID NO: 281), DR-hf2 (SEQ ID NO: 87), and DR2rc (SEQ ID NO: 81).

FIG. 19 shows the RNA secondary structure of the direct repeat sequence DR-hf2 (SEQ ID NO: 282) predicted by RNAfold.

### DETAILED DESCRIPTION

The present disclosure is further illustrated by the following embodiments, but does not thereby limit the present disclosure within the scope of these embodiments. Experimental methods for which specific conditions are not indicated in the following embodiments shall be selected in accordance with conventional methods and conditions, or in accordance with product instructions.

As used herein, the term “sequence identity” (identity or percent identity) is used to refer to the matching situation of sequences between two polypeptides or between two nucleic acids.

When a position in two sequences being compared is occupied by the same base or amino acid monomer subunit (e.g., a position in each of two DNA molecules is occupied by adenine, or a position in each of two polypeptides is occupied by lysine), then the molecules are identical at such position. The “percent identity” between two sequences is a function of the number of matched positions shared by the two sequences divided by the number of positions being compared ×100%. For example, if there are 6 matches in 10 positions of two sequences, the two sequences have 60% sequence identity. Typically, the comparison is made when aligning two sequences to produce maximum sequence identity. Such a comparison can be made by using disclosed and commercially available alignment algorithms and programs, such as but not limited to Clustal 22, MAFFT, Probcons, T-Coffee, Probalign, BLAST, which can be reasonably selected to use by a person skilled in the art. Those skilled in the art can determine suitable parameters for sequence alignment, such as any algorithms needed to achieve a superior alignment or optimal alignment for the full length of the compared sequences, and any algorithms needed to achieve a topical superior alignment or optimal alignment for the compared sequences.

As used herein, the term “guide polynucleotide” is used to refer to a molecule in the CRISPR-Cas system that forms a CRISPR complex with a Cas protein and guides the CRISPR complex to a target sequence. Typically, the guide polynucleotide comprises a scaffold sequence linked to the guide sequence, which can hybridize with the target sequence. The scaffold sequence usually comprises direct repeat sequences, and sometimes can also comprises a tracrRNA sequences. When the scaffold sequence does not comprise a tracrRNA sequence, the guide polynucleotide comprises both the guide sequence and the direct repeat sequence, in which case the guide polynucleotide can also be referred to as a crRNA.

### CRISPR-Cas13 System

Class 2 CRISPR-Cas systems endow microbes with diverse mechanisms for adaptive immunity.

Provided herein is an analysis of prokaryotic genomes and metagenomes to identify a previously uncharacterized RNA-guided, RNA-targeting CRISPR-Cas13 systems comprising C13-2 (also referred to as CasRfg.4), which is classified as a Type VI system. Engineered CRISPR-Cas13 systems based on C13-2 have robust activity in human cells. As a compact single effector Cas13 enzyme, C13-2 can also be flexibly packaged into an AAV vector. The results herein present C13-2 as a programmable RNA-binding module for efficient targeting of cellular RNA, thereby providing a general platform for transcriptome engineering and therapeutic and diagnostic methods.

As described in Example 1, a CRISPR-Cas13 system comprising C13-2 was identified based on bioinformatic analysis of prokaryotic genomes and metagenomes in NCBI GenBank and CNGB database, followed by experimental validation of targeted RNA cleavage activity in human cells.

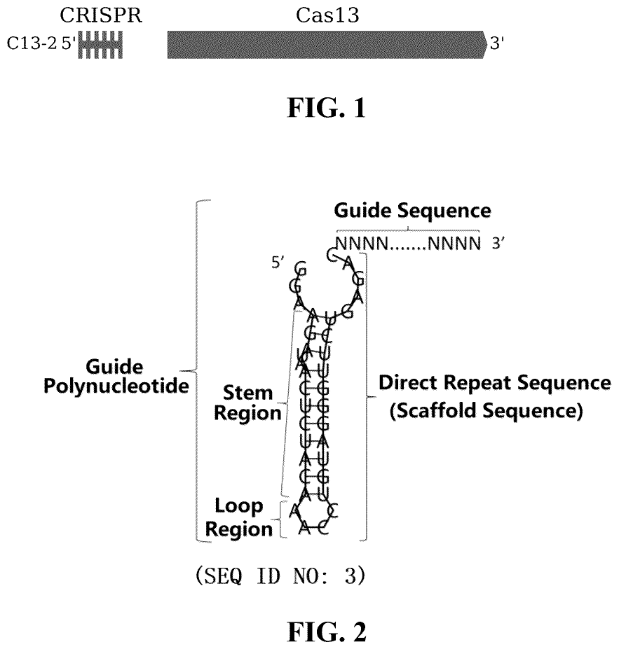

The CRISPR-Cas13 system comprising C13-2 is a Type-VI CRISPR-Cas system. FIG. 1 shows the CRISPR locus of the CRISPR-Cas13 system comprising C13-2. The protein sequence of the wild-type C13-2 is provided as SEQ ID NO: 1. The DNA coding sequence of the wild-type C13-2 is provided as SEQ ID NO: 9. FIG. 8 shows the in silico predicted structural domains of C13-2, which includes NTD, HEPN-1_I, HEPN-1_II, and HEPN-2, wherein NTD is the N terminus domain. The Helical-1 and Helical-2 domains are positioned between HEPN-1 I and HEPN-1_II, and between HEPN-1_II and HEPN-2, respectively. The direct repeat sequence of the C13-2 guide polynucleotide is provided as SEQ ID NO: 3. FIG. 2 shows the RNA secondary structure of the direct repeat sequence of the C13-2 guide polynucleotide as predicted by RNAfold. An engineered CRISPR-Cas13 system described herein can efficiently knockdown endogenous target RNAs in human cells, paving the way for RNA-targeting applications as part of a transcriptome engineering toolbox. In some embodiments, C13-2-mediated knockdown is capable of achieving higher efficiency and/or specificity relative to the knockdown mediated by CasRx, PspCas13b, Cas13X.1 and/or Cas13Y.1 across diverse endogenous transcripts.

Accordingly, one aspect of the disclosure provided herein relates to a CRISPR-Cas13 system, composition, or kit, comprising: a Cas13 protein or a fusion protein having at least 90% sequence identity compared to SEQ ID NO: 1, or a nucleic acid encoding the Cas13 protein, and a guide polynucleotide or a nucleic acid encoding the guide polynucleotide; wherein the guide polynucleotide comprises a direct repeat sequence linked to a guide sequence that is engineered to hybridize with the target RNA; and the guide polynucleotide is capable of forming a CRISPR complex with the Cas13 protein and guiding the sequence-specific binding of the CRISPR complex to the target RNA.

In some embodiments, the guide polynucleotide comprising a direct repeat sequence linked to a guide sequence that is engineered to hybridize with the target RNA; wherein the guide polynucleotide is capable of forming a CRISPR complex with the Cas13 protein and guiding the CRISPR complex to sequence-specifically bind to and cleave the target RNA.

In some embodiments, the polynucleotide sequence encoding the Cas13 protein or the fusion protein and/or the polynucleotide sequence encoding the guide polynucleotide is operably linked to a regulatory sequence. In some embodiments, the polynucleotide sequence encoding the Cas13 protein or the fusion protein is operably linked to a regulatory sequence. In some embodiments, the polynucleotide sequence encoding the guide polynucleotide is operably linked to a regulatory sequence. In some embodiments, the regulatory sequence of the polynucleotide sequence encoding the Cas13 protein or the fusion protein and the regulatory sequence of the polynucleotide sequence encoding the guide polynucleotide are the same or different.

In some embodiments, the Cas13 protein described herein comprises at least 90%, at least 91%, at least 92%, at least 93%, at least 94%, at least 95%, at least 96%, at least 97%, at least 98%, at least 99%, or at least 99.5% sequence identity compared to SEQ ID NO: 1. In some embodiments, the Cas13 protein described herein comprises at least 90%, at least 91%, at least 92%, at least 93%, at least 94%, at least 95%, at least 96%, at least 97%, at least 98%, or at least 99% sequence identity compared to the protein sequence encoded by SEQ ID NO: 9. When the CRISPR-Cas13 system comprises a fusion protein comprising the Cas13 protein, and a protein domain and/or a peptide tag, the percentage of sequence identity is calculated between the Cas13 portion of the fusion protein and the reference sequence.

In some embodiments, the Cas13 protein described herein derives from the species comprising a genome that has an average nucleotide identity (ANI) of ≥95% with the genome no.CNA0009596 in the CNGB database.

In some embodiments, the Cas 13 protein described herein comprises one or more (e.g., 1 or 2) native HEPN domains, each comprising an RX4H amino acid motif (wherein X indicates any amino acid, and the subscript “4” represents four consecutive amino acids). In some embodiments, a first catalytic RX4H motif is located at amino acid positions 210-215 of SEQ ID NO: 1, and a second catalytic RX4H motif is located at amino acid positions 785-790 of SEQ ID NO: 1, and a third RX4H motif is located at amino acid positions 750-755 of SEQ ID NO: 1. In some embodiments, the Cas13 protein described herein comprises one or more mutated HEPN domains. In some embodiments, the mutated Cas13 protein can process its guide polynucleotide, but cannot cleave the target RNA.

In some embodiments, the Cas13 protein described herein does not exhibit a protospacer flanking sequence (PFS) requirement for RNA cleavage.

The CRISPR-Cas13 system described herein can be introduced into cells (or to a cell-free system) in multiple non-limiting ways: (i) as Cas13 mRNA and guide polynucleotide, (ii) as part of a single vector or plasmid, or divided into multiple vectors or plasmids, (iii) as a separate Cas13 protein and guide polynucleotide, or (iv) as an RNP complex of the Cas13 protein and guide polynucleotide.

In some embodiments, the CRISPR-Cas13 system, composition, or kit comprises a nucleic acid molecule encoding the Cas13 protein, wherein the coding sequence is codon optimized for expression in a eukaryotic cell. In some embodiments, the CRISPR-Cas13 system, composition, or kit comprises a nucleic acid molecule encoding the Cas13 protein, wherein the coding sequence is codon optimized for expression in a mammalian cell. In some embodiments, the CRISPR-Cas13 system, composition, or kit comprises a nucleic acid molecule encoding the Cas13 protein, wherein the coding sequence is codon optimized for expression in a human cell.

In some embodiments, the nucleic acid molecule encoding the Cas13 protein is a plasmid. In some embodiments, the nucleic acid molecule encoding the Cas13 protein is part of the genome of a viral vector, such as the DNA genome of an AAV vector flanked by ITRs. In some embodiments, the nucleic acid molecule encoding the Cas13 protein is an mRNA.

### Guide Polynucleotide

In some embodiments, the guide polynucleotide of the CRISPR-Cas13 system is a guide RNA. In some embodiments, the guide polynucleotide is a chemically-modified guide polynucleotide. In some embodiments, the guide polynucleotide comprises at least one chemically-modified nucleotide. In some embodiments, the guide polynucleotide is a hybrid RNA-DNA guide. In some embodiments, the guide polynucleotide is a hybrid RNA-LNA (locked nucleic acid) guide.

In some embodiments, the guide polynucleotide comprises at least one guide sequence (also referred to as spacer sequence) linked to at least one direct repeat sequence (direct repeat, DR). In some embodiments, the guide sequence is positioned at the 3′ end of the direct repeat sequence. In some embodiments, the guide sequence is positioned at the 5′ end of the direct repeat sequence.

In some embodiments, the guide sequence comprises at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides. In some embodiments, the guide sequence comprises no more than 60 nucleotides, no more than 55 nucleotides, no more than 50 nucleotides, no more than 45 nucleotides, no more than 40 nucleotides, no more than 35 nucleotides, or no more than 30 nucleotides. In some embodiments, the guide sequence comprises 15-20 nucleotides, 20-25 nucleotides, 25-30 nucleotides, 30-35 nucleotides, or 35-40 nucleotides.

In some embodiments, the guide sequence has sufficient complementarity with the target RNA sequence to hybridize with the target RNA and guide the sequence-specific binding of the CRISPR-Cas13 complex to the target RNA. In some embodiments, the guide sequence has 100% complementarity with the target RNA (or region of the RNA to be targeted), but the guide sequence can have less than 100% complementarity with the target RNA, such as at least 80%, at least 85%, at least 90%, at least 95%, at least 98%, or at least 99% complementarity with the target RNA.

In some embodiments, the guide sequence is engineered to hybridize with the target RNA with no more than two mismatches. In some embodiments, the guide sequence is engineered to hybridize with the target RNA with no more than one mismatch. In some embodiments, the guide sequence is engineered to hybridize with the target RNA with or without a mismatch.

In some embodiments, the direct repeat sequence comprises at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, or at least 36 nucleotides. In some embodiments, the direct repeat comprises no more than 60 nucleotides, no more than 55 nucleotides, no more than 50 nucleotides, no more than 45 nucleotides, no more than 40 nucleotides, or no more than 35 nucleotides. In some embodiments, the direct repeat sequence comprises 20-25 nucleotides, 25-30 nucleotides, 30-35 nucleotides, or 35-40 nucleotides.

In some embodiments, the direct repeat sequence is modified to substitute at least one complementary base pair in the stem region shown in FIG. 2 with a different complementary base pair. In some embodiments, the direct repeat sequence is modified to vary the number of complementary base pairs in the stem region shown in FIG. 2. In some embodiments, the direct repeat sequence is modified to vary the number of nucleotides in the loop region shown in FIG. 2 (e.g., 3, 4, or 5 nucleotides in the loop). In some embodiments, the direct repeat sequence is modified to vary the nucleotide sequence in the loop region. In some embodiments, an aptamer sequence is inserted within or appended to the end of the direct repeat sequence. In some embodiments, the direct repeat sequence comprises at least 60%, at least 70%, at least 80%, at least 85%, at least 90%, at least 91%, at least 92%, at least 93%, at least 94%, at least 95%, at least 96%, at least 97%, at least 98%, at least 99%, or at least 100% sequence identity compared to any one of SEQ ID NO: 3 and SEQ ID NOs: 80-87.

In some embodiments, the CRISPR-Cas13 system, composition, or kit comprises at least 2, at least 3, at least 4, at least 5, at least 10, or at least 20 different guide polynucleotides. In some embodiments, the guide polynucleotides target at least 2, at least 3, at least 4, at least 5, at least 10, or at least 20 different target RNA molecules, or target at least 2, at least 3, at least 4, at least 5, at least 10, or at least 20 different regions of one or more target RNA molecules.

In some embodiments, the guide polynucleotide includes a constant direct repeat sequence positioned upstream of a variable guide sequence. In some embodiments, a plurality of guide polynucleotides are part of an array (which can be part of a vector, such as a viral vector or plasmid). For example, a guide array including the sequence DR-spacer-DR-spacer-DR-spacer, may include three unique unprocessed guide polynucleotides (one for each DR-spacer sequence). Once introduced into a cell or cell-free system, the array is processed by the Cas13 protein into three individual mature guide polynucleotides. This allows for multiplexing, e.g. the delivery of multiple guide polynucleotides to a cell or system to target multiple target RNAs or multiple regions within a single target RNA.

The ability of a guide polynucleotide to guide sequence-specific binding of a CRISPR complex to a target RNA may be assessed by any suitable assay. For example, the components of a CRISPR system sufficient to form a CRISPR complex, including the guide polynucleotide to be tested, may be provided to a host cell having the corresponding target RNA molecule, such as by transfection with vectors encoding the components of a CRISPR complex, followed by an assessment of preferential cleavage within the target sequence. Similarly, cleavage of a target RNA sequence may be evaluated in a test tube by providing the target RNA, components of a CRISPR complex, including the guide polynucleotide to be tested and a control guide polynucleotide different from the test guide polynucleotide, and comparing the binding ability or rate of cleavage at the target RNA between the test and control guide polynucleotide.

### Cas13 Mutants

In some embodiments, the Cas13 protein provided herein comprises one or more mutations, such as a single amino acid insertion, a single amino acid deletion, a single amino acid substitution, or combinations thereof, compared to wild-type C13-2 protein (SEQ ID NO: 1).

In some examples, the Cas13 protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 amino acid changes (such as insertion, deletion or substitution), compared to wild-type C13-2 protein (SEQ ID NO: 1), but retains the ability to bind target RNA molecules complementary to the guide sequence of the guide polynucleotide, and/or retains the ability to process an guide array RNA transcripts into guide polynucleotide molecules. In some examples, the Cas13 protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 amino acid changes (such as insertion, deletion or substitution), compared to wild-type C13-2 protein (SEQ ID NO: 1), but retains the ability to bind target RNA molecules complementary with the guide sequence of the guide polynucleotide.

In some examples, the Cas13 protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid changes (such as insertion, deletion or substitution), compared to wild-type C13-2 protein (SEQ ID NO: 1), but retains the ability to bind target RNA molecules complementary with the guide sequence of the guide polynucleotide, and/or retains the ability to process a guide array RNA transcript into guide polynucleotide molecules.

In some embodiments, the Cas13 protein comprises one or more mutations in the catalytic domain and has reduced RNA cleavage activity. In some embodiments, the Cas13 protein comprises one mutation in the catalytic domain and has reduced RNA cleavage activity. In some embodiments, the Cas13 protein comprises one or more mutations in one or both HEPN domains and substantially lacks RNA cleavage activity. In some embodiments, the Cas13 protein comprises a mutation in one or both HEPN domains and substantially lacks RNA cleavage activity. In some embodiments, the “substantially lack RNA cleavage activity” refers to the retention of ≤50%, ≤40%, ≤30%, ≤20%, ≤10%, ≤5%, or ≤1% of RNA cleavage activity compared to wild-type Cas13 protein, or the absence of detectable RNA cleavage activity.

In some embodiments, the Cas13 protein comprises at least 90%, at least 91%, at least 92%, at least 93%, at least 94%, at least 95%, at least 96%, at least 97%, at least 98%, or at least 99% sequence identity compared to SEQ ID NO: 1. In some embodiments, the Cas13 protein comprises at least 90%, at least 91%, at least 92%, at least 93%, at least 94%, at least 95%, at least 96%, at least 97%, at least 98%, or at least 99% sequence identity compared to the protein sequence encoded by SEQ ID NO: 9. When the CRISPR-Cas13 system comprises a fusion protein of the Cas13 protein with a protein domain and/or a peptide tag, the percentage of sequence identity is calculated between the Cas13 portion of the fusion protein and the reference sequence.

In some embodiments, the Cas13 protein can form a CRISPR complex with a guide polynucleotide, and the CRISPR complex is capable of sequence-specifically binding to the target RNA.

In some embodiments, the Cas13 protein is capable of forming a CRISPR complex with a guide polynucleotide comprising a direct repeat sequence linked to a guide sequence that is engineered to guide the sequence-specific binding of the CRISPR complex to the target RNA.

One type of modification or mutation includes the substitution of amino acids for amino acid residues having a similar biochemical property, i.e., a conservative substitution (such as conservative substitutions of 1-4, 1-8, 1-10, or 1-20 amino acids). Typically, the conservative substitution has little to no impact on the activity of the resulting protein or peptide.

For example, a conservative substitution is an amino acid substitution in a Cas13 protein that does not substantially affect the binding of the Cas13 protein to a target RNA molecule complementary with the guide sequence of the gRNA molecule, and/or the processing of the guide array RNA transcript into gRNA molecules. An alanine scan can be used to identify which amino acid residues in a Cas13 protein, can tolerate an amino acid substitutions. In one example, when an alanine or other conservative amino acid is substituted for 1-4, 1-8, 1-10, or 1-20 native amino acids, the ability of a variant Cas13 protein to modify gene expression in a CRISPR-Cas system is altered by no more than 25%, e.g., no more than 20%, e.g., no more than 10%. Examples of amino acids that may be substituted and are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

More substantial changes can be made by using substitutions that are less conservative, e.g., selecting residues that differ more significantly in maintaining the following effects: (a) the structure of the polypeptide backbone in the area of the substitution, e.g., as a helical or a sheet conformation; (b) the charge or hydrophobicity of the area interacting with the target site; or (c) the bulk of the side chain. The substitutions that in general are expected to produce the greatest changes in polypeptide function are: (a) a hydrophilic residue, e.g., serine or threonine, is substituted for (or by) a hydrophobic residue, e.g., leucine, isoleucine, phenylalanine, valine or alanine (b) a cysteine or proline is substituted for (or by) any other residue; (c) a residue having an electropositive side chain, e.g., lysine, arginine, or histidine, is substituted for (or by) an electronegative residue, e.g., glutamic acid or aspartic acid; or (d) a residue having a bulky side chain, e.g., phenylalanine, is substituted for (or by) one not having a side chain, e.g., glycine.

In some embodiments, the Cas13 protein comprises one or more mutations at amino acid positions 40-91 of SEQ ID NO: 1 (i.e., the region from the 40th amino acid to the 91st amino acid in the sequence of SEQ ID NO: 1, including the 40th amino acid and the 91st amino acid). In some embodiments, the Cas13 protein comprises one or more mutations at amino acid positions 146-153 of SEQ ID NO: 1. In some embodiments, the Cas13 protein comprises one or more mutations at amino acid positions 158-176 of SEQ ID NO: 1. In some embodiments, the Cas13 protein comprises one or more mutations at amino acid positions 182-209 of SEQ ID NO: 1. In some embodiments, the Cas13 protein comprises one or more mutations at amino acid positions 216-253 of SEQ ID NO: 1. In some embodiments, the Cas13 protein comprises one or more mutations at amino acid positions 271-287 of SEQ ID NO: 1. In some embodiments, the Cas13 protein comprises one or more mutations at amino acid positions 341-353 of SEQ ID NO: 1. In some embodiments, the Cas13 protein comprises one or more mutations at amino acid positions 379-424 of SEQ ID NO: 1. In some embodiments, the Cas13 protein comprises one or more mutations at amino acid positions 456-477 of SEQ ID NO: 1. In some embodiments, the Cas13 protein comprises one or more mutations at amino acid positions 521-557 of SEQ ID NO: 1. In some embodiments, the Cas13 protein comprises one or more mutations at amino acid positions 575-588 of SEQ ID NO: 1. In some embodiments, the Cas13 protein comprises one or more mutations at amino acid positions 609-625 of SEQ ID NO: 1. In some embodiments, the Cas13 protein comprises one or more mutations at amino acid positions 700-721 of SEQ ID NO: 1. In some embodiments, the Cas13 protein comprises one or more mutations at amino acid positions 724-783 of SEQ ID NO: 1. In some embodiments, the Cas13 protein comprises one or more mutations at amino acid positions 796-815 of SEQ ID NO: 1. In some embodiments, the Cas13 protein comprises one or more mutations at amino acid positions 828-852 of SEQ ID NO: 1. In some embodiments, the Cas13 protein comprises one or more mutations at amino acid positions 880-893 of SEQ ID NO: 1.

In some embodiments, the Cas13 protein comprises deletion of one or more amino acids at amino acid positions 348-350 of SEQ ID NO: 1 (i.e., the 348th amino acid, the 349th amino acid, and the 350th amino acid in the sequence of SEQ ID NO: 1). In some embodiments, the Cas13 protein comprises deletion of one or more amino acids at amino acid positions 521-556 of SEQ ID NO: 1. In some embodiments, the Cas13 protein comprises deletion of one or more amino acids at amino acid positions 883-893 of SEQ ID NO: 1.

In some embodiments, the RxxxxH motif of the Cas13 protein (x represents any amino acid, RxxxxH can also be referred to as Rx4H or R4xH) comprises one or more mutations and substantially lacks RNA cleavage activity.

In some embodiments, the Cas13 protein comprises a mutation at the position corresponding to the RxxxxH motifs at positions 210-215, 750-755, and/or 785-790 of the reference protein as shown in SEQ ID NO: 1.

In some embodiments, the Cas13 protein comprises a mutation at the position corresponding to the RxxxxH motif at positions 210-215 of the reference protein as shown in SEQ ID NO: 1. In some embodiments, the Cas13 protein comprises a mutation at the position corresponding to the RxxxxH motif at positions 750-755 of the reference protein as shown in SEQ ID NO: 1. In some embodiments, the Cas13 protein comprises a mutation at the position corresponding to the RxxxxH motif at positions 785-790 of the reference protein as shown in SEQ ID NO: 1.

In some embodiments, the Cas13 protein comprises a mutation at the position corresponding to the RxxxxH motifs at positions 210-215 and 750-755 of the reference protein as shown in SEQ ID NO: 1.

In some embodiments, the Cas13 protein comprises a mutation at the position corresponding to the RxxxxH motifs at positions 210-215 and 785-790 of the reference protein as shown in SEQ ID NO: 1.

In some embodiments, the Cas13 protein comprises a mutation at the position corresponding to the RxxxxH motifs at positions 750-755 and 785-790 of the reference protein as shown in SEQ ID NO: 1.

In some embodiments, the Cas13 protein comprises a mutation at the position corresponding to the RxxxxH motifs at positions 210-215, 750-755 and 785-790 of the reference protein as shown in SEQ ID NO: 1.

In some embodiments, the RxxxxH motif is mutated to AxxxxxH, RxxxxA, or AxxxxxA. In some embodiments, the RxxxxH motif is mutated to AxxxxxH. In some embodiments, the RxxxxH motif is mutated to RxxxxA. In some embodiments, the RxxxxH motif is mutated to AxxxxxA.

In some embodiments, the Cas13 protein comprises 1, 2, 3, 4, 5, or 6 mutations at the position corresponding to the amino acid residues R210, H215, R750, H755, R785, and/or H790 of the reference protein as shown in SEQ ID NO: 1. In some embodiments, the amino acid residues of the Cas13 protein at the position corresponding to the amino acid residues R210, H215, R750, H755, R785, and/or H790 of the reference protein as shown in SEQ ID NO: 1 is mutated to A (alanine).

In some embodiments, the Cas13 protein comprises a mutation at the position corresponding to the amino acid residues R210 and H215 of the reference protein as shown in SEQ ID NO: 1. In some embodiments, the Cas13 protein comprises a mutation at the position corresponding to the amino acid residues R750 and H755 of the reference protein as shown in SEQ ID NO: 1. In some embodiments, the Cas13 protein comprises a mutation at the position corresponding to the amino acid residues R785 and H790 of the reference protein as shown in SEQ ID NO: 1.

In some embodiments, the Cas13 protein comprises a mutation at the position corresponding to the amino acid residues R210, H215, R750 and H755 of the reference protein as shown in SEQ ID NO: 1.

In some embodiments, the Cas13 protein comprises a mutation at the position corresponding to the amino acid residues R750, H755, R785 and H790 of the reference protein as shown in SEQ ID NO: 1.

In some embodiments, the Cas13 protein comprises a mutation at the position corresponding to the amino acid residues R210, H215, R785 and H790 of the reference protein as shown in SEQ ID NO: 1.

In some embodiments, the Cas13 protein comprises a mutation at the position corresponding to the amino acid residues R210, H215, R750, H755, R785 and H790 of the reference protein as shown in SEQ ID NO: 1.

In some embodiments, the position corresponding to the amino acid residue R210, R750, or R785 is mutated to A. In some embodiments, the position corresponding to the amino acid residue H215, H755, or H790 is mutated to A. In some embodiments, the positions corresponding to the amino acid residues R210, H215, R750, H755, R785, and H790 are all mutated to A.

In some embodiments, the Cas13 protein is obtained by introducing a mutation in the RxxxxH motifs at the position 210-215, 750-755, and/or 785-790 of the sequence as shown in SEQ ID NO: 1.

In some embodiments, the Cas13 protein is obtained by introducing 1, 2, 3, 4, 5, or 6 mutations at the position R210, H215, R750, H755, R785, and/or H790 of the sequence as shown in SEQ ID NO: 1. In some embodiments, the Cas13 protein is obtained by mutating the amino acid residues at the position R210, H215, R750, H755, R785, and/or H790 of the sequence as shown in SEQ ID NO: 1 to A (alanine).

In some embodiments, the Cas13 protein is obtained by mutating the amino acid residues at the position R210, H215, R785, and H790 of the sequence as shown in SEQ ID NO: 1 to A. In some embodiments, the Cas13 protein is obtained by mutating the amino acid residues at the position R210, H215, R750, and H755 of the sequence as shown in SEQ ID NO: 1 to A. In some embodiments, the Cas13 protein is obtained by mutating the amino acid residues at the position R750, H755, R785, and H790 of the sequence as shown in SEQ ID NO: 1 to A. In some embodiments, the Cas13 protein is obtained by mutating the amino acid residues at the position R210, H215, R750, H755, R785, and H790 of the sequence as shown in SEQ ID NO: 1 to A.

In some embodiments, the Cas13 protein comprises at least one mutation at the position corresponding to amino acid residue positions 40-91, 146-153, 158-176, 182-209, 216-253, 271-287, 341-353, 379-424, 456-477, 521-557, 575-588, 609-625, 700-721, 724-783, 796-815, 828-852 or 880-893 of the reference protein as shown in SEQ ID NO: 1. In some embodiments, the Cas13 protein comprises deletion of one or more amino acids at the position corresponding to amino acid residue positions 348-350, 521-556, or 883-893 of the reference protein as shown in SEQ ID NO: 1.

In some embodiments, compared with the reference protein as shown in SEQ ID NO: 1, the Cas13 protein comprises any one or more mutations at the position corresponding to the following amino acid residues of the reference protein as shown in SEQ ID NO: 1: R11, N34, R35, R47, R58, R63, R64, N68, N87, N265, N274, R276, R290, R294, N299, N303, R308, R314, R320, R328, N332, R341, N346, R358, N372, N383, N390 N394, R47+R290, R47+R314, R290+R314, R47+R290+R314, R308+N68, N394+N68, N87+N68, R308+N265, N394+N265, N87+N265, R308+N68+N265, N87+N68+N265, T7, A16, S260, A263, M266, N274, F288, M302, N303, L304, V305, 1311, D313, H324, P326, H327, N332, N346, T353, T360, E365, A373, M380, S382, K395, Y396, D402, D411, S418.

In some embodiments, compared with the reference protein as shown in SEQ ID NO: 1, the position of the Cas13 protein corresponding to the mutation sites in Table 24 of the reference protein as shown in SEQ ID NO: 1 is mutated to the same amino acid residue. In some embodiments, compared with the reference protein as shown in SEQ ID NO: 1, the Cas13 protein comprises the same mutation at the positions corresponding to the mutation sites in Table 24 of the reference protein as shown in SEQ ID NO: 1.

In some embodiments, the Cas13 protein is obtained by introducing any one or more mutations at the following positions of the sequence as shown in SEQ ID NO: 1: R11, N34, R35, R47, R58, R63, R64, N68, N87, N265, N274, R276, R290, R294, N299, N303, R308, R314, R320, R328, N332, R341, N346, R358, N372, N383, N390, N394, R47+R290, R47+R314, R290+R314, R47+R290+R314, R308+N68, N394+N68, N87+N68, R308+N265, N394+N265, N87+N265, R308+N68+N265, N87+N68+N265, T7, A16, S260, A263, M266, N274, F288, M302, N303, L304, V305, 1311, D313, H324, P326, H327, N332, N346, T353, T360, E365, A373, M380, S382, K395, Y396, D402, D411, S418.

In some embodiments, the Cas13 protein is obtained by introducing any one or more mutations in Table 24 to the sequence as shown in SEQ ID NO: 1.

In some embodiments, the Cas13 protein is obtained by sequence deletion at the position corresponding to amino acid residue positions 91-120, 141-180, 211-240, 331-360, 351-400, 431-460, 461-500, 511-550, 611-640, 631-660, 661-690, 691-760, 821-860, or 861-890 of the reference protein as shown in SEQ ID NO: 1.

In some embodiments, ≤300, ≤200, ≤150, ≤100, ≤90, ≤80, ≤70, ≤60, ≤50, ≤40, ≤30, ≤20, or ≤10 amino acid residues are deleted in the sequence deletion.

In some embodiments, the Cas13 protein is obtained by sequence deletion at positions 91-120, 141-180, 211-240, 331-360, 351-400, 431-460, 461-500, 511-550, 611-640, 631-660, 661-690, 691-760, 821-860, or 861-890 of the sequence as shown in SEQ ID NO: 1.

Subcellular Localization Signal (or referred to as Localization Signal)

In some embodiments, the Cas13 protein or the functional fragment thereof is fused to at least one homologous or heterologous subcellular localization signal. Exemplary subcellular localization signals include organelle localization signals, such as a nuclear localization signal (NLS), a nuclear export signal (NES) or a mitochondrial localization signal.

In some embodiments, the Cas13 protein or the functional fragment thereof is fused to at least one homologous or heterologous NLS. In some embodiments, the Cas13 protein or the functional fragment thereof is fused to at least two NLSs. In some embodiments, the Cas13 protein or the functional fragment thereof is fused to at least three NLSs. In some embodiments, the Cas13 protein or the functional fragment thereof is fused to at least one N-terminus NLS and at least one C-terminus NLS. In some embodiments, the Cas13 protein or the functional fragment thereof is fused to at least two C-terminus NLSs. In some embodiments, the Cas13 protein or the functional fragment thereof is fused to at least two N-terminus NLSs.

In some embodiments, the NLS is independently selected from SPKKKRKVEAS (SEQ ID NO: 53), GPKKKRKVAAA (SEQ ID NO: 54), PKKKRKV (SEQ ID NO: 55), KRPAATKKAGQAKKKK (SEQ ID NO: 56), PAAKRVKLD (SEQ ID NO: 57), RQRRNELKRSP (SEQ ID NO: 58) NQSSNFGPMKGGNFGGRSSGPYGGGGQYFAKPRNQGGY (SEQ ID NO: 59), RMRIZFKNKGKDTAELRRRRVEVSVELRKAKKDEQILKRRNV (SEQ ID NO: 60), VSRKRPRP (SEQ ID NO: 61), PPKKARED (SEQ ID NO: 62), POPKKKPL (SEQ ID NO: 63), SALIKKKKKMAP (SEQ ID NO: 64), DRLRR (SEQ ID NO: 65), PKQKKRK (SEQ ID NO: 66), RKLKKKIKKL (SEQ ID NO: 67), REKKKFLKRR (SEQ ID NO: 68), KRKGDEVDGVDEVAKKKSKK (SEQ ID NO: 69), or RKCLQAGMNLEARKTKK (SEQ ID NO: 70), and PAAKKKKLD (SEQ ID NO: 71).

In some embodiments, the Cas13 protein or the functional fragment thereof is fused to at least one homologous or heterologous NES. In some embodiments, the Cas13 protein or the functional fragment thereof is fused to at least two NESs. In some embodiments, the Cas13 protein or the functional fragment thereof is fused to at least three NESs. In some embodiments, the Cas13 protein or the functional fragment thereof is fused to at least one N-terminus NES and at least one C-terminus NES. In some embodiments, the Cas13 protein or the functional fragment thereof is fused to at least two C-terminus NESs. In some embodiments, the Cas13 protein or the functional fragment thereof is fused to at least two N-terminus NESs.

In some embodiments, the NES is independently selected from an adenovirus type 5 E1B NES, an HIV Rev NES, a MAPK NES, and a PTK2 NES.

In some embodiments, the Cas13 protein or the functional fragment thereof is fused to homologous or heterologous NLS and NES, wherein a cleavable linker is positioned between the NLS and the NES. In some embodiments, the NES facilitates the production of delivery particles (e.g., viral-like particles) comprising the Cas13 protein or the functional fragment thereof in a production cell line. In some embodiments, cleavage of the linker in a target cell may expose the NLS and facilitate nuclear localization of the Cas13 protein or the functional fragment thereof in the target cell.

### Protein Domain and Polypeptide Tag

In some embodiments, the Cas13 protein or the functional fragment thereof is covalently linked or fused to a homologous or heterologous protein domain and/or a polypeptide tag. In some embodiments, the Cas13 protein or the functional fragment thereof is fused to a homologous or heterologous protein domain and/or a polypeptide tag.

In some embodiments, the protein domain and polypeptide tag are optionally selected from: a cytosine deaminase domain, an adenosine deaminase domain, a translational activation domain, a translational repression domain, an RNA methylation domain, an RNA demethylation domain, a ribonuclease domain, a splicing factor domain, a reporter domain, an affinity domain, a subcellular localization signal, a reporter tag, and an affinity tag.

In some embodiments, the protein domain comprises a cytosine deaminase domain, an adenosine deaminase domain, a translational activation domain, a translational repression domain, an RNA methylation domain, an RNA demethylation domain, a ribonuclease domain, a splicing factor domain, a reporter domain, and an affinity domain. In some embodiments, the polypeptide tag comprises a reporter tag and an affinity tag.

In some embodiments, the length of the amino acid sequence of the protein domain is ≥40 amino acids, ≥50 amino acids, ≥60 amino acids, ≥70 amino acids, ≥80 amino acids, ≥90 amino acids, ≥100 amino acids, ≥150 amino acids, ≥200 amino acids, ≥250 amino acids, ≥300 amino acids, ≥350 amino acids, or ≥400 amino acids.

Exemplary protein domains include those can cleave RNA (e.g., a PIN endonuclease domain, an NYN domain, an SMR domain from SOT1, or an RNase domain from Staphylococcal nuclease), those can affect RNA stability (e.g., tristetraprolin (TTP) or domains from UPF1, EXOSC5, and STAU1), those can edit a nucleotide or ribonucleotide (e.g., a cytidine deaminase, PPR protein, adenosine deaminase, ADAR family protein, or APOBEC family protein), those can activate translation (e.g., eIF4E and other translation initiation factors, a domain of the yeast poly(A)-binding protein or GLD2), those can inhibit translation (e.g., Pumilio or FBF PUF proteins, deadenylases, CAF1, Argonaute proteins), those can methylate RNA (e.g., domains from m6A methyltransferase factors such as METTL14, METTL3, or WTAP), those can demethylate RNA (e.g., human alkylation repair homolog 5), those can affect splicing (e.g., the RS-rich domain of SRSF1, the Gly-rich domain of hnRNP A1, the alanine-rich motif of RBM4, or the proline-rich motif of DAZAP1), those can enable affinity purification or immunoprecipitation, and those can enable proximity-based protein labeling and identification (e.g., a biotin ligase (such as BirA) or a peroxidase (such as APEX2) in order to biotinylate proteins that interact with the target RNA).

In some embodiments, the protein domain comprises an adenosine deaminase domain. In some embodiments, a Cas13 protein described herein with a mutated HEPN domain, a catalytically inactive Cas13 protein, or the functional fragment thereof is covalently linked or fused to an adenosine deaminase domain to guide A-to-I deaminase activity on RNA transcripts in mammalian cells. Adenosine deaminase domains engineered based on ADAR2 for targeting A-to-I RNA editing are described in Cox et al., Science 358 (6366): 1019-1027 (2017), which is incorporated herein by reference in its entirety. In other embodiments, the adenosine deaminase domain is covalently linked or fused to an adaptor protein which is capable of binding to an aptamer sequence inserted within or appended to the guide polynucleotide, thereby allowing the adenosine deaminase domain to be non-covalently linked to the Cas13 protein or the functional fragment thereof that is complexed with the guide polynucleotide.

In some embodiments, the protein domain comprises a cytosine deaminase domain. In some embodiments, a Cas13 protein described herein with a mutated HEPN domain, a catalytically inactive Cas13 protein, or the functional fragment thereof is covalently linked or fused to a cytosine deaminase domain to guide C-to-U deaminase activity on RNA transcripts in mammalian cells. Cytosine deaminase domains evolved from ADAR2 for targeting C-to-U RNA editing are described in Abudayyeh et al., Science 365 (6451): 382-386 (2019), which is incorporated herein by reference in its entirety. In other embodiments, the cytosine deaminase domain is covalently linked or fused to an adaptor protein which is capable of binding to an aptamer sequence inserted within or appended to the guide polynucleotide, thereby allowing the cytosine deaminase domain to be non-covalently linked to the Cas13 protein or the functional fragment thereof that is complexed with the guide polynucleotide.

In some embodiments, the protein domain comprises a splicing factor domain. In some embodiments, a Cas13 protein described herein with a mutated HEPN domain, a catalytically inactive Cas13 protein, or the functional fragment thereof is covalently linked or fused to a splicing factor domain to guide alternative splicing of the target RNA in mammalian cells. Splicing factor domains for targeting selective splicing are described in Konermann et al., Cell 173 (3): 665-676 (2018), which is incorporated herein by reference in its entirety. Non-limiting examples of the splicing factor domain include RS-rich domain of SRSF1, Gly-rich domain of hnRNPA1, alanine-rich motif of RBM4, or proline-rich motif of DAZAP1. In other embodiments, the splicing factor domain is covalently linked or fused to an adaptor protein which is capable of binding to an aptamer sequence inserted within or appended to the guide polynucleotide, thereby allowing the splicing factor domain to be non-covalently linked to the Cas13 protein or the functional fragment thereof that is complexed with the guide polynucleotide.

In some embodiments, the protein domain comprises a translational activation domain. In some embodiments, a Cas13 protein described herein with a mutated HEPN domain, a catalytically inactive Cas13 protein, or the functional fragment thereof is covalently linked or fused to a translational activation domain for activating or increasing expression of the target RNA. Non-limiting examples of the translational activation domain include eIF4E and other translation initiation factors, a domain of the yeast poly(A)-binding protein or GLD2. In other embodiments, the translational activation domain is covalently linked or fused to an adaptor protein which is capable of binding to an aptamer sequence inserted within or appended to the guide polynucleotide, thereby allowing the translational activation domain to be non-covalently linked to the Cas13 protein or the functional fragment thereof that is complexed with the guide polynucleotide.

In some embodiments, the protein domain comprises a translational repression domain. In some embodiments, a Cas13 protein described herein with a mutated HEPN domain, a catalytically inactive Cas13 protein, or the functional fragment thereof is covalently linked or fused to a translational repression domain for repressing or decreasing expression of the target RNA. Non-limiting examples of the translational repression domain include Pumilio or FBF PUF proteins, deadenylases, CAF1, Argonaute proteins. In other embodiments, the translational repression domain is covalently linked or fused to an adaptor protein which is capable of binding to an aptamer sequence inserted within or appended to the guide polynucleotide, thereby allowing the translational repression domain to be non-covalently linked to the Cas13 protein or the functional fragment thereof that is complexed with the guide polynucleotide.

In some embodiments, the protein domain comprises an RNA methylation domain. In some embodiments, a Cas13 protein described herein with a mutated HEPN domain, a catalytically inactive Cas13 protein, or the functional fragment thereof is covalently linked or fused to a RNA methylation domain for methylation of the target RNA. Non-limiting examples of the RNA methylation domain include m6A domains such as METTL14, METTL3, or WTAP. In other embodiments, the RNA methylation domain is covalently linked or fused to an adaptor protein which is capable of binding to an aptamer sequence inserted within or appended to the guide polynucleotide, thereby allowing the RNA methylation domain to be non-covalently linked to the Cas13 protein or the functional fragment thereof that is complexed with the guide polynucleotide.

In some embodiments, the protein domain comprises an RNA demethylation domain. In some embodiments, a Cas13 protein described herein with a mutated HEPN domain, a catalytically inactive Cas13 protein, or the functional fragment thereof is covalently linked or fused to a RNA demethylation domain for demethylation of the target RNA. Non-limiting examples of the RNA methylation domain include human alkylation repair homolog 5 or ALKBH5. In other embodiments, the RNA demethylation domain is covalently linked or fused to an adaptor protein which is capable of binding to an aptamer sequence inserted within or appended to the guide polynucleotide, thereby allowing the RNA demethylation domain to be non-covalently linked to the Cas13 protein or the functional fragment thereof that is complexed with the guide polynucleotide.

In some embodiments, the protein domain comprises a ribonuclease domain. In some embodiments, a Cas13 protein described herein with a mutated HEPN domain, a catalytically inactive Cas13 protein, or the functional fragment thereof is covalently linked or fused to a ribonuclease domain for cleavage of the target RNA. Non-limiting examples of the ribonuclease domain include a PIN endonuclease domain, an NYN domain, an SMR domain from SOT1, or an RNase domain from Staphylococcal nuclease.

In some embodiments, the protein domain comprises an affinity domain or reporter domain. In some embodiments, a Cas13 protein or the functional fragment thereof described herein is covalently linked or fused to a reporter domain such as a fluorescent protein. Non-limiting examples of the reporter domain include GST, HRP, CAT, GFP, HcRed, DsRed, CFP, YFP, or BFP.

In some embodiments, a Cas13 protein described herein is covalently linked or fused to a polypeptide tag. In some embodiments, an example of the polypeptide tag is a small polypeptide sequence. In some embodiments, the length of the amino acid sequence of the polypeptide tag is ≤50 amino acids, <40 amino acids, ≤30 amino acids, ≤25 amino acids, ≤20 amino acids, ≤15 amino acids, ≤10 amino acids, or ≤5 amino acids. In some embodiments, a Cas13 protein described herein is covalently linked or fused to an affinity tag such as a purification tag. Non-limiting examples of the affinity tag include HA-tag, His-tag (such as 6-His), Myc-tag, E-tag, S-tag, calmodulin tag, FLAG-tag, GST-tag, MBP-tag, Halo tag, or biotin.

In some embodiments, the dead C13-2 mutant (R210A+H215A+R750A+H755A+R785A+H790A) is fused to a protein domain and/or a peptide tag.

In some embodiments, the Cas13 protein is fused to ADAR.

In some embodiments, the dead C13-2 mutant (R210A+H215A+R750A+H755A+R785A+H790A) is fused to a cytosine deaminase or an adenine deaminase.

In some embodiments, the dead C13-2 mutant (R210A+H215A+R750A+H755A+R785A+H790A) is covalently linked to a cytosine deaminase or an adenine deaminase directly, by rigid linker peptide sequence A (EAAAK) 3A, or by flexible linker peptide sequence (GGGGS) 3.

### Aptamer Sequence

In some embodiments, the guide polynucleotide further comprises an aptamer sequence.

In some embodiments, the aptamer sequence is inserted into a loop of the guide polynucleotide.

In some embodiments, the aptamer sequence is inserted into the tetra loop of the guide polynucleotide. An example of tetra loop of the guide polynucleotide is shown in FIG. 2. In some embodiments, the aptamer sequence is appended to an end of the guide polynucleotide.

Insertion of an aptamer sequence into a guide polynucleotide of the CRISPR-Cas system is described in Konermann et al., Nature 517:583-588 (2015), which is incorporated herein by reference in its entirety. In some embodiments, the aptamer sequence includes an MS2 aptamer sequence, a PP7 aptamer sequence, or a QB aptamer sequence.

### Adaptor Protein

In some embodiments, the CRISPR-Cas13 system further comprises a fusion protein comprising an adaptor protein and a homologous or heterologous protein domain and/or a polypeptide tag, or a nucleic acid encoding the fusion protein, wherein the adaptor protein is capable of binding to the aptamer sequence.

Fusion proteins of an adaptor protein and a protein domain are described in Konermann et al., Nature 517:583-588 (2015), which is incorporated herein by reference in its entirety. In some embodiments, the adaptor protein includes an MS2 phage coat protein (MCP), a PP7 phage coat protein (PCP), or a QB phage coat protein (QCP). In some embodiments, the protein domain comprises a cytosine deaminase domain, an adenosine deaminase domain, a translational activation domain, a translational repression domain, an RNA methylation domain, an RNA demethylation domain, a nuclease domain, a splicing factor domain, an affinity domain or a reporter domain.

### Modified Guide Polynucleotide

In some embodiments, the guide polynucleotide comprises a modified nucleotide. In some embodiments, the modified nucleotide comprises 2′-O-methyl, 2′-O-methyl-3′phosphorothioate, or 2′-O-methyl-3′thioPACE. In some embodiments, the guide polynucleotide is a chemically-modified guide polynucleotide. Chemically-modified guide polynucleotide is described in Hendel et al., Nat. Biotechnol. 33 (9): 985-989 (2015), which is incorporated herein by reference in its entirety.

In some embodiments, the guide polynucleotide is a hybrid RNA-DNA guide, a hybrid RNA-LNA (locked nucleic acid) guide, a hybrid DNA-LNA guide, or a hybrid DNA-RNA-LNA guide. In some embodiments, the direct repeat sequence comprises one or more ribonucleotides substituted with corresponding deoxyribonucleotides. In some embodiments, the guide sequence comprises one or more ribonucleotides substituted with corresponding deoxyribonucleotides. Hybrid RNA-DNA guide polynucleotides are described in WO2016/123230, which is incorporated herein by reference in its entirety.

### Vector System

Another aspect of the disclosure provided herein relates to a vector system comprising the CRISPR-Cas13 system described herein, wherein the vector system comprises one or more vectors comprising a polynucleotide sequence encoding the Cas13 protein and a polynucleotide sequence encoding the guide polynucleotide.

In some embodiments, the vector system comprises at least one plasmid or viral vector (e.g., retrovirus, lentivirus, adenovirus, adeno-associated virus, or herpes simplex virus). In some embodiments, the polynucleotide sequence encoding the Cas13 protein or fusion protein and the polynucleotide sequence encoding the guide polynucleotide are located on the same vector. In some embodiments, the polynucleotide sequence encoding the Cas13 protein or fusion protein and the polynucleotide sequence encoding the guide polynucleotide are located on a plurality of vectors.

In some embodiments, the polynucleotide sequence encoding the Cas13 protein or fusion protein and/or the polynucleotide sequence encoding the guide polynucleotide is operably linked to a regulatory sequence. In some embodiments, the polynucleotide sequence encoding the Cas13 protein or fusion protein is operably linked to a regulatory sequence. In some embodiments, the polynucleotide sequence encoding the guide polynucleotide is operably linked to a regulatory sequence. In some embodiments, the regulatory sequence of the polynucleotide sequence encoding a Cas13 protein or fusion protein and the regulatory sequence of the polynucleotide sequence encoding a guide polynucleotide are the same or different. In some embodiments, the regulatory sequence is optionally selected from promoters, enhancers, internal ribosomal entry sites (IRES), and other expression control elements (e.g., transcription termination signals, such as polyadenylation signals and poly-U sequences). In some embodiments, the regulatory sequence includes the regulatory sequence that enables the constitutive expression of the nucleotide sequence in many types of host cells, as well as the regulatory sequence that enables the expression of the nucleotide sequence only in certain host cells (e.g., tissue-specific regulatory sequence). A tissue-specific promoter can be directly expressed primarily in a desired tissue of interest, such as muscle, neuron, bone, skin, blood, specific organs (e.g., liver, pancreas), or particular cell types (e.g., lymphocytes). Regulatory elements can also direct expression in a temporal-dependent manner, such as in a cell-cycle dependent or developmental stage-dependent manner, which may or may not be tissue or cell-type specific. In some embodiments, the regulatory sequence is an enhancer element such as WPRE, a CMV enhancer, an R-U5′ segment in LTR of HTLV-I, a SV40 enhancer, or an intron sequence between exons 2 and 3 of rabbit β-globin.

In some embodiments, the vector comprises a pol III promoter (e.g., U6 and H1 promoters), a pol II promoter (e.g., the retroviral Rous sarcoma virus (RSV) LTR promoter (optionally with the RSV enhancer), a cytomegalovirus (CMV) promoter (optionally with the CMV enhancer), a SV40 promoter, a dihydrofolate reductase promoter, a β-actin promoter, a phosphoglycerol kinase (PGK) promoter, or an EFla promoter), or a pol III promoter and a pol II promoter.

In some embodiments, the promoter is a constitutive promoter that is continuously active and is not subject to regulation by external signals or molecules. Suitable constitutive promoters include, but are not limited to CMV, RSV, SV40, EFla, CAG, and beta-actin. In some embodiments, the promoter is an inducible promoter that is regulated by an external signal or molecule (e.g., a transcription factor).

In some embodiments, the promoter is a tissue-specific promoter, which can be used to drive tissue-specific expression of the Cas13 protein or fusion protein. Suitable muscle specific promoters include, but are not limited to CK8, MHCK7, Myoglobin promoter (Mb), Desmin promoter, muscle creatine kinase promoter (MCK) and variants thereof, and SPc5-12 synthetic promoter. Suitable immune cell specific promoters include, but are not limited to, B29 promoter (B cells), CD14 promoter (monocytic cells), CD43 promoter (leukocytes and platelets), CD68 (macrophages), and SV40/CD43 promoter (leukocytes and platelets). Suitable blood cell specific promoters include, but are not limited to, CD43 promoter (leukocytes and platelets), CD45 promoter (hematopoietic cells), INF-β (hematopoietic cells), WASP promoter (hematopoietic cells), SV40/CD43 promoter (leukocytes and platelets), and SV40/CD45 promoter (hematopoietic cells). Suitable pancreatic specific promoters include, but are not limited to, Elastase-1 promoter. Suitable endothelial cell specific promoters include, but are not limited to, Fit-1 promoter and ICAM-2 promoter. Suitable neuronal tissue/cell specific promoters include, but are not limited to, GFAP promoter (astrocytes), SYN1 promoter (neurons), and NSE/RU5′ (mature neurons). Suitable kidney specific promoters include, but are not limited to, NphsI promoter (podocytes). Suitable bone specific promoters include, but are not limited to, OG-2 promoter (osteoblasts, odontoblasts). Suitable lung specific promoters include, but are not limited to, SP-B prompter (lung). Suitable liver specific promoters include, but are not limited to, SV40/Alb promoter. Suitable heart specific promoters include, but are not limited to, α-MHC.

### AAV Vector

Another aspect of the disclosure provided herein relates to an adeno-associated viral (AAV) vector comprising the CRISPR-Cas13 system described herein, wherein the AAV vector comprises a DNA encoding the Cas13 protein or the fusion protein and the guide polynucleotide.

Delivery of a CRISPR-Cas system by an AAV vector is described in Maeder et al., Nature Medicine 25:229-233 (2019), which is incorporated herein by reference in its entirety. In some embodiments, the AAV vector comprises an ssDNA genome comprising coding sequences for the Cas13 protein or the fusion protein and the guide polynucleotide flanked by ITRs.

In some embodiments, the CRISPR-Cas13 system described herein is packaged in an AAV vector, such as AAV1, AAV2, AAV3, AAV4, AAV5, AAV6, AAV7, AAV8, AAV9, and AAVrh74. In some embodiments, the CRISPR-Cas13 system described herein is packaged in an AAV vector comprising an engineered capsid having tissue-tropism, such as an engineered muscle-tropism capsid. Engineering of AAV capsids with tissue-tropism by directed evolution is described in Tabebordbar et al., Cell 184:4919-4938 (2021), which is incorporated herein by reference in its entirety.

### Lipid Nanoparticle

Another aspect of the disclosure provided herein relates to a lipid nanoparticle (LNP) comprising the CRISPR-Cas13 system described herein, wherein the LNP comprises the guide polynucleotide described herein and an mRNA encoding the Cas13 protein or the fusion protein described herein.

LNP delivery of CRISPR-Cas systems is described in Gillmore et al., N. Engl. J. Med., 385:493-502 (2021), which is incorporated herein by reference in its entirety. In some embodiments, in addition to the RNA payload (Cas13 mRNA and guide polynucleotide), the LNP comprises four components: a cationic or ionizable lipid, cholesterol, a helper lipid, and a PEG-lipid. In some embodiments, the cationic or ionizable lipid comprises cKK-E12, C12-200, ALC-0315, DLin-MC3-DMA, DLin-KC2-DMA, FTT5, Moderna SM-102, and Intellia LP01. In some embodiments, the PEG-lipid comprises PEG-2000-C-DMG, PEG-2000-DMG, or ALC-0159. In some embodiments, the helper lipid comprises DSPC. Components of LNPs are described in Paunovska et al., Nature Reviews Genetics 23:265-280 (2022), which is incorporated herein by reference in its entirety.

### Lentiviral Vector

Another aspect of the disclosure provided herein relates to a lentiviral vector comprising the CRISPR-Cas13 system described herein, wherein the lentiviral vector comprises the guide polynucleotide described herein and the mRNA encoding the Cas13 protein or the fusion protein described herein. In some embodiments, the lentiviral vector is pseudotyped with a homologous or heterologous envelope protein such as VSV-G. In some embodiments, the mRNA encoding the Cas13 protein or the fusion protein is linked to an aptamer sequence.

### RNP Complex

Another aspect of the disclosure provided herein relates to a ribonucleoprotein complex comprising the CRISPR-Cas13 system described herein, wherein the ribonucleoprotein complex is formed by the guide polynucleotide and the Cas13 protein or the fusion protein described herein. In some embodiments, the ribonucleoprotein complex can be delivered to eukaryotic cells, mammalian cells, or human cells by microinjection or electroporation. In some embodiments, the ribonucleoprotein complex can be packaged in a viral-like particle and delivered to a mammalian or human subject in vivo.

### Viral-Like Particle

Another aspect of the disclosure provided herein relates to a viral-like particle (VLP) comprising the CRISPR-Cas13 system described herein, wherein the viral-like particle comprises the guide polynucleotide and the Cas13 protein or the fusion protein described herein; or a ribonucleoprotein complex formed by the guide polynucleotide and the Cas13 protein or the fusion protein.

Engineered VLPs are described in Banskota et al., Cell 185 (2): 250-265 (2022), Mangeot et al., Nature Communications 10 (1): 1-15 (2019), Campbell, et al., Molecular Therapy 27:151-163 (2019), Campbell, et al., Molecular Therapy, 27 (2019): 151-163, and Mangeot et al., Molecular Therapy, 19 (9): 1656-1666 (2011), which are incorporated herein by reference in its entirety. In some embodiments, the engineered VLP is pseudotyped with a homologous or heterologous envelope protein such as VSV-G. In some embodiments, the Cas13 protein is fused to a gag protein (e.g., MLVgag) by a cleavable linker, wherein the cleavage of the linker in the target cell exposes a NLS positioned between the linker and the Cas13 protein. In some embodiments, the fusion protein comprises (e.g., from 5′ to 3′) a gag protein (e.g., MLVgag), one or more NESs, a cleavable linker, one or more NLSs, and a Cas13 protein, such as described in Banskota et al. Cell 185 (2): 250-265 (2022). In some embodiments, the Cas13 protein is fused to a first dimerizable domain that is capable of dimerization or heterodimerization with a second dimerizable domain fused to a membrane protein, wherein the presence of a ligand facilitates said dimerization and enriches the Cas13 protein or the fusion protein into the VLP, such as described in Campbell, et al., Molecular Therapy 27:151-163 (2019).

### Cells

Another aspect of the disclosure provided herein relates to a cell comprising the CRISPR-Cas13 system described herein. The cell (e.g., which can be used to generate a cell-free system) can be eukaryotic or prokaryotic. Examples of such cells include, but are not limited to bacteria, archaea, plant, fungal, yeast, insect, and mammalian cells, such as Lactobacillus, Lactococcus, Bacillus (such as B. subtilis), Escherichia (such as E. coli), Clostridium, Saccharomyces or Pichia (such as S. cerevisiae or P. pastoris), Kluyveromyces lactis, Salmonella typhimurium, Drosophila cells, C. elegans cells, Xenopus cells, SF9 cells, C129 cells, 293 cells, Neurospora, and immortalized mammalian cell lines (e.g., Hela cells, myeloid cell lines, and lymphoid cell lines).

In some embodiments, the cell is a prokaryotic cell, such as a bacterial cell, such as E. coli. In some embodiments, the cell is a eukaryotic cell, such as a mammalian cell or a human cell. In some embodiments, the cell is a primary eukaryotic cell, a stem cell, a tumor/cancer cell, a circulating tumor cell (CTC), a blood cell (e.g., T cell, B cell, NK cell, Tregs, etc.), a hematopoietic stem cell, a specialized immune cell (e.g., tumor-infiltrating lymphocyte or tumor-suppressed lymphocytes), a stromal cell in the tumor microenvironment (e.g., cancer-associated fibroblasts, etc.). In some embodiments, the cell is a brain or neuronal cell of the central or peripheral nervous system (e.g., neurons, astrocytes, microglia cells, retinal ganglion cells, rod/cone cells, etc.).

### Target RNA Molecule

The CRISPR-Cas13 system, composition, or kit described herein can be used to target one or more target RNA molecules, such as the target RNA molecules present in a biological sample, an environmental sample (e.g., soil, air or water sample), etc. In some embodiments, the target RNA is a coding RNA such as a pre-mRNA or a mature mRNA. In some embodiments, the target RNA is a nuclear RNA. In some embodiments, the target RNA is an RNA transcript located in the nucleus of a eukaryotic cell. In some embodiments, the target RNA is a non-coding RNA such as functional RNA, siRNA, microRNA, snRNA, snoRNA, piRNA, scaRNA, tRNA, rRNA, lncRNA, or lincRNA.

In some embodiments, in addition to targeting the target RNA molecule, the CRISPR-Cas13 system, composition, or kit described herein performs one or more of the following functions on the target RNA: cleaving one or more target RNA molecules or nicking one or more target RNA molecules, activating or upregulating one or more target RNA molecules, activating or inhibiting translation of one or more target RNA molecules, deactivating one or more target RNA molecules, visualizing, labeling, or detecting one or more target RNA molecules, binding one or more target RNA molecules, editing one or more target RNA molecules, transporting one or more target RNA molecules, and masking one or more target RNA molecules. In some examples, the CRISPR-Cas13 system, composition, or kit described herein modifies one or more target RNA molecules, the modifying one or more target RNA molecules including one or more of the following: an RNA base substitution, an RNA base deletion, an RNA base insertion, a break in the target RNA, an RNA methylation, and an RNA demethylation. In some embodiments, the CRISPR-Cas13 system, composition, or kit described herein can target one or more target RNA molecules. In some embodiments, the CRISPR-Cas13 system, composition, or kit described herein can bind one or more target RNA molecules. In some embodiments, the CRISPR-Cas13 system, composition, or kit described herein can cleave one or more target RNA molecules. In some embodiments, the CRISPR-Cas13 system, composition, or kit described herein can activate translation of one or more target RNA molecules. In some embodiments, the CRISPR-Cas13 system, composition, or kit described herein can inhibit translation of one or more target RNA molecules. In some embodiments, the CRISPR-Cas13 system, composition, or kit described herein can detect one or more target RNA molecules. In some embodiments, the CRISPR-Cas13 system, composition, or kit described herein can edit one or more target RNA molecules.

In some embodiments, the target RNA is AQp1 RNA. Knocking-down the level of AQp1 RNA using the CRISPR-Cas13 system described herein can reduce the production of aqueous humor and lowers intraocular pressure, which can be used for treatment of diseases such as glaucoma. In some embodiments, the target RNA is AQp1 RNA, and the guide sequence of the guide polynucleotide is SEQ ID NO: 5.

In some embodiments, the target RNA is PTBP1 RNA. Knocking-down the level of PTBP1 RNA using the CRISPR-Cas13 system described herein can facilitate the transdifferentiation of brain astrocytes into neurons, which can be used for treatment of diseases such as Parkinson's disease. In some embodiments, the target RNA is PTBP1 RNA, and the guide sequence of the guide polynucleotide is SEQ ID NO: 6.

In some embodiments, the target RNA is VEGFA RNA. Knocking-down the level of VEGFA RNA using the CRISPR-Cas13 system described herein can prevent choroidal neovascularization, which can be used for treatment of diseases such as age-related macular degeneration.

In some embodiments, the target RNA is ANGPTL3 RNA. Knocking-down the level of ANGPTL3 RNA using the CRISPR-Cas13 system described herein can lower blood lipids such as low-density lipoprotein cholesterol (LDL-C), which can be used for treatment of atherosclerotic cardiovascular diseases such as hyperlipidemia and familial hypercholesterolemia. In some embodiments, the target RNA is ANGPTL3 RNA, and the guide sequence of the guide polynucleotide is selected from any one or more of SEQ ID NOs: 42-49. Therapeutic applications

A further aspect of the disclosure provided herein relates to a pharmaceutical composition comprising the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particles described herein, or the eukaryotic cells described herein. The pharmaceutical composition can comprises, for example, an AAV vector encoding the Cas13 protein or the fusion protein and the guide polypeptide described herein. The pharmaceutical composition can comprises, for example, a lipid nanoparticle comprising the guide polypeptide described herein and an mRNA encoding the Cas13 protein or the fusion protein. The pharmaceutical composition can comprises, for example, a lentiviral vector comprising the guide polypeptide described herein and an mRNA encoding the Cas13 protein or the fusion protein. The pharmaceutical composition can comprises, for example, a viral-like particle comprising the guide polynucleotide described herein and the Cas13 protein or the fusion protein; or a ribonucleoprotein complex formed by the guide polynucleotide and the Cas13 protein or the fusion protein.

Another aspect of the disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in cleaving and editing the target RNA in a mammalian cell.

Another aspect of the disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in any one of the following: cleaving one or more target RNA molecules or nicking one or more target RNA molecules; activating or upregulating one or more target RNA molecules; activating or inhibiting translation of one or more target RNA molecules; inactivating one or more target RNA molecules; visualizing, labelling or detecting one or more target RNA molecules; binding one or more target RNA molecules; transporting one or more target RNA molecules; and masking one or more target RNA molecules.

Another aspect of the disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in modifying one or more target RNA molecules in a mammalian cell, wherein the modifying one or more target RNA molecules comprises one or more of the following: an RNA base substitution, an RNA base deletion, an RNA base insertion, a break in the target RNA, an RNA methylation, and an RNA demethylation.

Another aspect of the disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in diagnosing, treating, or preventing a disease or disorder associated with the target RNA. In some embodiments, the disease or disorder is Parkinson's disease. In some embodiments, the disease or disorder is Parkinson's disease, and the target RNA is PTBP1 RNA. In some embodiments, the disease or disorder is glaucoma. In some embodiments, the disease or disorder is glaucoma, and the target RNA is AQp1 RNA. In some embodiments, the disease or disorder is amyotrophic lateral sclerosis. In some embodiments, the disease or disorder is amyotrophic lateral sclerosis, and the target RNA is superoxide dismutase 1 (SOD1) RNA. In some embodiments, the disease or disorder is age-related macular degeneration, and the target RNA is VEGFA RNA. In some embodiments, the disease or disorder is age-related macular degeneration, and the target RNA is VEGFA RNA or VEGFR1 RNA. In some embodiments, the disease or disorder is increase of plasma LDL cholesterol level. In some embodiments, the disease or disorder is increase of plasma LDL cholesterol level, and the target RNA is PCSK9 RNA or ANGPTL3 RNA.

Another aspect of the disclosure provided herein relates to a use of the CRISPR-Cas13 system described herein, the Cas13 protein described herein, the fusion protein described herein, the guide polynucleotide described herein, the nucleic acid described herein, the vector system described herein, the lipid nanoparticle described herein, the lentiviral vector described herein, the ribonucleoprotein complex described herein, the viral-like particle described herein, or the eukaryotic cell described herein in the manufacture of a medicaments for diagnosing, treating, or preventing a disease or disorder associated with the target RNA. In some embodiments, the disease or disorder is Parkinson's disease. In some embodiments, the disease or disorder is glaucoma. In some embodiments, the disease or disorder is amyotrophic lateral sclerosis. In some embodiments, the disease or disorder is age-related macular degeneration. In some embodiments, the disease or disorder is increase of plasma LDL cholesterol level. In some embodiments, the disease or disorder is Parkinson's disease, and the target RNA is PTBP1 RNA. In some embodiments, the disease or disorder is glaucoma, and the target RNA is AQp1 RNA. In some embodiments, the disease or disorder is amyotrophic lateral sclerosis, and the target RNA is superoxide dismutase 1 (SOD1) RNA. In some embodiments, the disease or disorder is age-related macular degeneration, and the target RNA is VEGFA RNA or VEGFR1 RNA. In some embodiments, the disease or disorder is increase of plasma LDL cholesterol level, and the target RNA is PCSK9 RNA or ANGPTL3 RNA.

In some embodiments, the pharmaceutical composition is delivered to a human subject in vivo. The pharmaceutical composition can be delivered by any effective route. Exemplary routes of administration include, but are not limited to, intravenous infusion, intravenous injection, intraperitoneal injection, intramuscular injection, intratumoral injection, subcutaneous injection, intradermal injection, intraventricular injection, intravascular injection, intracerebellar injection, intraocular injection, subretinal injection, intravitreal injection, intracameral injection, intratympanic injection, intranasal administration, and inhalation.

In some embodiments, the method for targeting an RNA results in editing the sequence of a target RNA. For example, by using a Cas13 protein or fusion protein with a non-mutated HEPN domain, and a guide polynucleotide comprising a guide sequence specific for the target RNA, the target RNA can be cleaved or nicked at a precise location (nick, e.g. cleaving either single strand when the target RNA is present as a double-stranded nucleic acid molecule). In some examples, such a method is used to decrease expression of a target RNA, which will decrease translation of the corresponding protein. Such a method can be used in a cell where increased expression of an RNA is not desired. In one example, the RNA is associated with a disease such as cystic fibrosis, Huntington's disease, Tay-Sachs, Fragile X syndrome, Fragile X-associated tremor/ataxia syndrome, muscular dystrophy, myotonic dystrophy, spinal muscular atrophy, spinocerebellar ataxia, age-related macular degeneration, or familial ALS. In another example, the RNA is associated with cancer (e.g., lung cancer, breast cancer, colon cancer, liver cancer, pancreas cancer, prostate cancer, bone cancer, brain cancer, skin cancer (such as melanoma) or kidney cancer). Examples of target RNAs include, but are not limited to, those associated with cancer (e.g., PD-L1, BCR-ABL, Ras, Raf, p53, BRCA1, BRCA2, CXCR4, β-catenin, HER2, and CDK4). Editing such target RNAs can have therapeutic effects.

In some embodiments, the RNA is expressed in an immune cell. For example, the target RNA can encode for a protein leading to the repression of a desirable immune response (e.g., tumor infiltration). Knocking down of such an RNA could enable progression of such a desirable immune response (e.g., PD1, CTLA4, LAG3, TIM3). In another example, the target RNA encodes a protein resulting in the undesirable activation of an immune response, for example in the context of an autoimmune disease such as multiple sclerosis, Crohn's disease, lupus, or rheumatoid arthritis.

### Diagnostic Applications

Another aspect of the disclosure provided herein relates to an in vitro composition comprising the CRISPR-Cas13 system described herein, and a labeled detector RNA that is not hybridizable with the guide polynucleotide described herein.

A further aspect of the disclosure provided herein relates to use of the CRISPR-Cas13 system described herein in the detection of a target RNA in a nucleic acid sample suspected of comprising the target RNA.

In some embodiments, the method for detecting RNA including a Cas13 protein or the fusion protein fused to a fluorescent protein or other detectable labels along with a guide polynucleotide comprising a guide sequence specific for the target RNA. Binding of a Cas13 protein or the fusion protein to the target RNA can be visualized by microscopy or other methods of imaging.

In another example, RNA aptamer sequences can be appended to or inserted within the guide polynucleotide, such as MS2, PP7, QB, and other aptamers. The introduction of proteins that specifically bind to these aptamers, e.g., the MS2 phage coat protein fused to a fluorescent protein or other detectable labels can be used to detect the target RNA, because the Cas13-guide-target RNA complex will be labeled by the aptamer interaction.

In some embodiments, the method for detecting a target RNA in a cell-free system results in a detectable label or enzyme activity. For example, by using a Cas13 protein, a guide polynucleotide comprising a guide sequence specific for the target RNA, and a detectable label, the target RNA will be recognized by Cas13. The binding of the target RNA by Cas13 triggers its RNase activity, which can lead to the cleavage of the target RNA as well as the detectable label.

In some embodiments, the detectable label is an RNA linked to a fluorescent probe and quencher. The intact detectable RNA links the fluorescent probe and quencher, suppresses fluorescence. Upon cleavage by Cas13 of the detectable RNA, the fluorescent probe is released from the quencher and displays fluorescent activity. Such a method can be used to determine if a target RNA is present in a lysed cell sample, lysed tissue sample, blood sample, saliva sample, environmental sample (e.g. a water, soil, or air sample), or other lysed cells or cell-free sample.

Such a method can also be used to detect a pathogen, such as a virus or bacteria, or to diagnose a disease state, e.g. a cancer.

In some embodiments, the detection of the target RNA aids in the diagnosis of a disease and/or pathological state, or the existence of a viral or bacterial infection. For example, Cas13 mediated detection of non-coding RNAs such as PCA3 can be used to diagnose prostate cancer if detected in the patient's urine. In another example, Cas13 mediated detection of the lncRNA-AA174084, which is a biomarker of gastric cancer, can be used to diagnose gastric cancer.

### EXAMPLES

### Example 1: Screening of C13-2 Protein

### 1. Annotation for CRISPR and Gene

The microbial genome from NCBI GenBank and CNGB (China National GenBank) databases was used to predict whole-genomic proteins by a software, followed with the prediction of CRISPR Array in genome by a software.

### 2. Preliminary Screening of Protein

Redundant proteins were removed by clustering, while filtering out proteins with amino acid sequence lengths less than 800 aa (amino acids) or greater than 1400 aa.

### 3. Acquisition of CRISPR-associated Protein

The protein sequence within 10 kb upstream and downstream of the CRISPR Array were compared with the sequence of a known Cas13, and the proteins with an e-value greater than 1*e-5 were filtered out. Then, by comparing with the NR library of NCBI, and the patent library of EBI, and proteins with high similarity were filtered out. Candidate proteins were obtained via further selection. By experimental verification, C13-2 protein was finally obtained (SEQ ID NO: 1, 893 aa). C13-2 protein is also referred to as CasRfg.4 protein.

The source of genomic sequence of the C13-2 protein is shown in Table 1.

TABLE 1
The source of genomic sequence of the C13-2 protein

| | | | Position of corresponding coding | Annotation on the source |
|---|---|---|---|---|
| Protein | Database | Genome No. | sequence in the genome | of species in the database |
| C13-2 | CNGB | CNA0009596 | F4453_scaffolds_778:1543:4224:+ | metagenome |

The DNA coding sequence of natural (wild-type) C13-2 protein is as shown in SEQ ID NO: 9.

The structure of gene locus of C13-2 proteins is as shown in FIG. 1, comprising CRISPR Array and C13-2 coding sequence.

The direct repeat (DR) sequence or scaffold sequence used in combination with C13-2 could be: 5′-GGAAGAUAACUCUACAAACCUGUAGGGUUCUGAGAC-3′ (SEQ ID NO: 3).

The RNA secondary structure of the said direct repeat sequence was predicted by RNAfold and presented in FIG. 2.

### Example 2: Preparation, Isolation and Purification of C13-2 Proteins

### I. Plasmid Construction

1. pET28a vector plasmid was taken, cleaved by double enzyme digestion via BamHI and Xhol, and subjected to agarose gel electrophoresis. A linearized vector was recovered by cutting the gel, a DNA fragment containing the coding sequence of a protein (which encodes the C13-2 protein and the nuclear localization sequence) was inserted into the cloning area of the vector pET28a by homologous recombination, and the vector was transformed into Stb13 competent by a reaction solution, coated onto an LB plates containing kanamycin sulfate, and incubated overnight at 37° C., and clones were picked for sequencing and identification.

The constructed recombinant vector was named C13-2-pET28a (SEQ ID NO: 10). The recombinant vector was used for expressing a C13-2 recombinant protein (SEQ ID NO: 11). The architecture of the recombinant C13-2 protein was His tag-NLS-Cas13-SV40 NLS-nucleoplasmin NLS.

2. Positive clones with correct sequences were incubated overnight, subjected to plasmid extraction, then transformed into an expression strain RIPL-BL21 (DE3), coated onto an LB plates containing kanamycin sulfate, and incubated overnight at 37° C.

### II. Protein Expression

1. Monoclones were picked and plated into 5 mL LB culture solution containing kanamycin sulfate, and incubated overnight at 37° C.

2. They were transferred into 500 mL of LB culture solution containing kanamycin sulfate at the volume ratio of 1:100, cultured at 220 rpm at 37° C. until the OD value was 0.6, added with IPTG to a final concentration of 0.2 mM, and induced at 16° C. for 24 h.

3. Collection of bacteria by centrifuging: the bacteria were rinsed with 15 mL PBS, then centrifuged for collection, added with a lysis buffer for ultrasonic crushing, and centrifuged at 10,000 g for 30 min to obtain a supernatant containing the recombinant protein, and the supernatant was filtered through a 0.45 μm filter membrane before purification on a column.

### III. Protein Purification

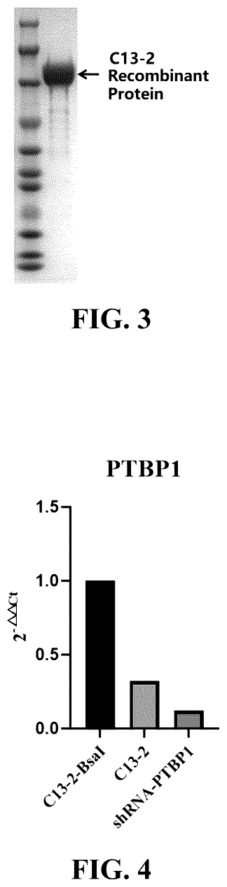

The purification was performed by MAC (Ni Sepharose 6 Fast Flow, CYTIVA®) and HITRAP HEPARIN HP (CYTIVA®). Upon SDS-PAGE electrophoresis, the purified C13-2 recombinant protein was shown in a band (see FIG. 3).

### Example 3: Verification of Editing Efficacy on an Exogenous Gene

### 1. Synthesis of the Vector to be Verified and Control Vector Targeting EGFP

The sequence for synthesis of expression vectors expressing foreign EGFP was as shown in SEQ ID NO: 13, and the structure of the plasmid was CMV-EGFP. The plasmid of the verified vector of C13-2 protein targeting EGFP was synthesized, with the full-length sequence as shown in SEQ ID NO: 14. The structure of the plasmid was CMV-C13-2-U6-gRNA.

EGFP was used as an exogenous reporter gene, and its nucleic acid sequence (720 bp) was as shown in SEQ ID NO: 12.

The guide sequence targeting EGFP was: ugccguucuucugcuugucggccaugauau (SEQ ID NO: 4).

2. Transfection of a 293T Cell with the Vector to be Verified

The plasmid expressing the exogenous EGFP was transfected into a 293T cell in a 24-well plate with the plasmid of the verified vector of C13-2 protein targeting EGFP at a ratio of 1:2 (166 ng: 334 ng).

The transfection method was as follows:

1. The 293T cells were digested by trypsin (0.25% of Trypsin, EDTA, Thermo, 25200056), counted, and plated into a 24-well plates at 2×10⁵ cells according to 500 μL per well.

2. For each transfected sample, the complex was prepared according to the following steps:
a. Each well of the 24-well plate into which the cells were added, was added with 50 μL of serum-free Opti-MEM™MI (Thermo, 11058021) reduced serum medium for dilution of the aforementioned plasmid DNA (500 ng), and mixed gently.
b. It was gently mixed with Lipofectamine™ 2000 (Thermo, 11668019) before use, and then 1 μL of the Lipofectamine™ 2000 was diluted in each well, i.e., in 50 μL of the Opti-MEM™ I medium. It was incubated at room temperature for 5 minutes. Note: it was continued to perform step c within 25 minutes.
c. After incubation for 5 minutes, the diluted DNA was combined with the diluted Lipofectamine™ 2000. They were gently mixed and incubated at room temperature for 20 minutes (the solution might be cloudy visually). Note: the complex was stabilized at room temperature for 6 hours. The complex was added into the 293T cells and mixed, and detected by a flow cytometer after 48 h.

### 3. Detection of the Down-Regulation Effect on EGFP Expression by Flow Cytometer

The description of cells and plasmids as used was as shown in Table 2 below:

TABLE 2
Grouping for Experiments

| Group for | |
|---|---|
| experiments | Notes |
| 293T | Cell vector control |
| EGFP | Transfected with expression vector of exogenous EGFP |
| C13-2 | Transfected with expression vector of EGFP and verified |
| | vector of C13-2 targeting EGFP |

At 48 h after transfection, the cells obtained were digested with trypsin (0.25% of Trypsin, EDTA, Thermo), and centrifuged at 300 g for 5 min, the supernatant was discarded, and the cells in each well were resuspended with 500 μL of PBS. The EGFP fluorescent expression was detected by a flow cytometer, wherein the cell debris were removed by FCS-A and SSC-A gating, and then detection was conducted by the flow cytometer and the data was collected.

The Mean-FITC-A results of the FITC channel were collected and recorded, and the down-regulation amplitude was calculated according to the following calculation formula:
wherein the GFP fluorescence of the EGFP group was a, and the GFP fluorescence of other groups was x. Down-regulation amplitude (%)=(a-x)+a ×100, wherein the blank control group did not participate in the comparison. The down-regulation amplitude results were as shown in Table 3, wherein the expression was down-regulated with 64.49% with C13-2 targeting EGFP.

TABLE 3
Results of GFP fluorescence as detected by a flow cytometer

| | Down-regulation amplitude of |
|---|---|
| Group | GFP fluorescence (%) |
| 293T | N/A |
| EGFP | 0.00 |
| C13-2 | 64.49 |

### Example 4: Verification of Editing Efficacy on An Endogenous Gene

I. Construction of vectors targeting AQp1 and PTBP1

The plasmid for expression vector C13-2-BsaI was synthesized, wherein the sequence is as shown in SEQ ID NO: 15.

The target nucleic acid selected in the experiment were AQp1 (Aquaporin 1) and PTBP1 (Polypyrimidine Tract Binding Protein 1), wherein AQp1 was verified by using a 293T cell line with high expression of AQp1, and PTBP1 was verified by using a 293T cell line.

The method for constructing the 293T cell line with high expression of Aqp1 (293T-AQp1 cell): a vector Lv-AQp1-T2α-GFP with over-expression of the AQP1 gene and the EGFP gene (SEQ ID NO: 16) was constructed. AQp1 and EGFP were spaced apart by a 2A peptide. The Lv-AQp1-T2a-GFP plasmid was packaged into a lentivirus and transduced into 293T cells to form a cell line stably overexpressing AQp1 gene.

The guide sequence of an AQp1-targeting
gRNA was:
(SEQ ID NO: 5)
agggcagaaccgaugcugaugaagac.
The guide sequence of an PTBP1-targeting
gRNA was:
(SEQ ID NO: 6)
GUGGUUGGAGAACUGGAUGUAGAUGGGCUG.

A target site-targeted fragment was obtained by using a primer annealing manner, wherein the primers were as follows:

PTBP1-targeting:
Forward Primer:
(SEQ ID NO: 22)
5′-AGACGTGGTTGGAGAACTGGATGTAGATGGGCTG-3′
Reverse Primer:
(SEQ ID NO: 23)
5′-AAAACAGCCCATCTACATCCAGTTCTCCAACCAC-3′
AQp1-targeting:
Forward Primer:
(SEQ ID NO: 24)
5′-AGACagggcagaaccgatgctgatgaagac-3′
Reverse Primer:
(SEQ ID NO: 23)
5′-AAAAgtcttcatcagcatcggttctgccct-3′

The primer annealing reaction system was as follows: it was incubated in a PCR instrument at 95° C. for 5 minutes, then immediately taken out and incubated on ice for 5 minutes, so that the primers were annealed to each other to form a double-stranded DNA with sticky ends.

| | |
|---|---|
| Oligo-F (10 μM) | 2 μL |
| Oligo-R (10 μM) | 2 μL |
| 10× endonuclease reaction buffer* | 2 μL |
| Deionized water | up to 20 μL |

After the synthesized Cas13-2-BsaI vector plasmid was digested with a Bsa I endonuclease, the annealed products and the backbones purified and recovered after the digestion were subjected to linkage with a T4 DNA ligase. After the transformation into Escherichia coli, the positive clones were selected and the plasmids were extracted. C13-2 vector (vector plasmids of C13-2 targeting AQp1 or PTBP1) was obtained for the following C13-2 experimental group.

The structure of C13-2 vector was CMV-C13-2U6-gRNA, and the vector could be used to express C13-2 protein and gRNA targeting Aqp1 or PTBP1.

The following control vectors were prepared by conventional methods:

CasRx-AQp1 plasmid, the positive control vector of CasRx targeting AQp1, with the sequence of which was as shown in SEQ ID NO: 17 and the structure of which was CMV-CasRx-U6-gRNA.

The plasmid comprises the sequence encoding CasRx (the amino acid sequence was as shown in SEQ ID NO: 2).

shRNA-AQp1 plasmid, the positive control vector of shRNA targeting AQp1, with the sequence of which was as shown in SEQ ID NO: 19, was used to express shRNA molecule. The sequence of the shRNA molecule was:

(SEQ ID NO: 7)
CCACGACCCUCUUUGUCUUCACUCGAGUGAAGACAAAGAGGGUCGUGG.

shRNA-PTBP1 plasmid, the positive control vector of shRNA targeting PTBP1, with the sequence of which was as shown in SEQ ID NO: 20, was used to express shRNA molecule. The sequence of the shRNA molecule was:

(SEQ ID NO: 8)
CAGCCCAUCUACAUCCAGUUCCUCGAGGAACUGGAUGUAGAUGGGCUG.

CasRx-blank plasmid, the blank control vector, could express CasRx and gRNA, where the gRNA did not targets AQp1 or PTBP1. The sequence of the plasmid was as shown in SEQ ID NO: 21, and the structure of the plasmid was CMV-CasRx-U6-gRNA.

II. Transfection of 293T Cells and 293T-AQp1 Cells with the Vector to be Verified

293T-AQp1 cells were transfected with the control plasmids or vector plasmids targeting AQp1 at 500 ng in a 24-well plate. 293T cells were transfected with the control plasmids or vector plasmids targeting PTBP1 at 500 ng in a 24-well plate.

The transfection method was as follows:

1. The cells were digested by trypsin (0.25% of Trypsin, EDTA, Thermo), counted, and plated into a 24-well plates at 2×10⁵ cells according to 500 μL per well.

2. For each transfected sample, the complex was prepared according to the following steps:
a. Each well of the 24-well plate into which the cells were added, was added with 50 μL of serum-free Opti-MEM™ I (Thermo, 25200056) reduced serum medium for dilution of the aforementioned plasmid DNA, and mixed gently.
b. Lipofectamine™ 2000 (Thermo, 11668019) was gently mixed before use, and then 1.8 μL of Lipofectamine™ 2000 was diluted in each well, i.e., in 50 μL of the Opti-MEM™ I medium. It was incubated at room temperature for 5 minutes. Note: it was continued to perform step c within 25 minutes.
c. After incubation for 5 minutes, the diluted DNA was combined with the diluted Lipofectamine™ 2000. They were gently mixed and incubated at room temperature for 20 minutes (the solution might be cloudy visually). Note: the complex was stabilized at room temperature for 6 hours.

The complex was added into the cells and mixed.

III. Detection of the RNA of the Target Gene by qPCR

At 48 h after transfection, the cells were subjected to RNA extraction with a SteadyPure Universal RNA Extraction Kit AG21017 kit, and the RNA concentration was detected with an ultramicro spectrophotometer. The RNA product was reverse transcribed by using an Evo M-MLV Mix Kit with gDNA Clean for qPCR reverse transcription kit, and the reverse transcribed product was detected by using a SYBR® Green Premix Pro Taq HS qPCR Kit.

Primers used in the qPCR were as follows:

Detection of PTBP1:
Forward Primer:
(SEQ ID NO: 26)
5′-ATTGTCCCAGATATAGCCGTTG-3′
Reverse Primer:
(SEQ ID NO: 27)
5′-GCTGTCATTTCCGTTTGCTG-3′
Detection of AQp1:
Forward Primer:
(SEQ ID NO: 28)
5′-gctcttctggagggcagtgg-3′
Reverse Primer:
(SEQ ID NO: 29)
5′-cagtgtgacagccgggttgag-3′
Detection of internal reference GAPDH:
Forward primer:
(SEQ ID NO: 30)
5′-CCATGGGGAAGGTGAAGGTC-3′
Reverse primer:
(SEQ ID NO: 31)
5′-GAAGGGGTCATTGATGGCAAC-3′

A reaction system was configured according to the instructions of the SYBR® Green Premix Pro Taq HS qPCR Kit, and detected by using a QuantStudio™ 5 Real-Time PCR System.

In this experiment, the target RNA was calculated by using a relative quantitative method, namely a 2^{-ΔΔCt} method.

The calculation method was as follows:

$$Δ C t = Ct ( AQp 1 ) - Ct ( GAPDH ) or Ct ( PTBP 1 ) - Ct ( GAPDH )$$

$$ΔΔ Ct = Δ Ct ( a sample to be verified , such as C 13 - 2 ) - Δ Ct ( CasRx - blank or C 13 - 2 - BsaI )$$

$${2}^{- Δ Δ C t} = 2 \hat{} ( - ΔΔ Ct ) .$$

The amount of RNA of AQp1 and PTBP1 were calculated according to the aforementioned calculation method. For verification experiment targeting PTBP1, the results were as shown in Table 4 and FIG. 4. For verification targeting AQp1, the experiments were conducted in triplicate as independent biological experiments (the transfection operation was conducted in 293T cells of the same batch), and the average results of the triplicate were obtained, the results of which were as shown in Table 5 and FIG. 5.

TABLE 4
The knockdown test results of PTBP1 RNA

| Groups for experiments | PTBP1 RNA level |
|---|---|
| C13-2-BsaI | 1.00 |
| C13-2 | 0.32 |
| shRNA-PTBP1 | 0.12 |

TABLE 5
Knockdown test results of AQp1 RNA

| | AQp1 RNA level | | | |
|---|---|---|---|---|
| Group for | Experiment | Experiment | Experiment | |
| experiments | 1 | 2 | 3 | Average |
| CasRx-blank | 1.00 | 1.00 | 1.00 | 1.00 |
| CasRx | 0.04 | 0.04 | 0.20 | 0.09 |
| shRNA-AQp1 | 0.10 | 0.23 | 0.30 | 0.21 |
| C13-2-BsaI | — | 0.97 | — | 0.97 |
| C13-2 | 0.02 | 0.00 | 0.00 | 0.01 |

Note:
“—” represents for no detection.
C13-2-BsaI and CasRx-blank were control groups without targeting AQp1 or PTBP1.

The experimental results demonstrated that in combination with the gRNA of the present example, C13-2 showed obvious editing efficiency, wherein the editing activity targeting PTBP1 was high while the editing activity targeting AQp1 was higher than CasRx.

### Example 5: Editing Efficiency Comparison with Disclosed Cas13 Proteins

### I. Construction of an Editing Vector Targeting Endogenous Gene PTBP1

Patent U.S. Pat. No. 10,476,825B2 discloses the Cas13 protein from BMZ-11B_GL0037771, and the Cas13 protein was named as C13-113 (the amino acid sequence of said protein was as shown in SEQ ID NO: 32 herein) in the present example. The direct repeat sequence used in this example corresponding to C13-113 was as shown in SEQ ID NO: 33.

Cas protein MBR0191107.1 was disclosed in GenBank, named as C13-114 (the amino acid sequence of this protein was as shown in SEQ ID NO: 34 herein). The direct repeat sequence used in this example corresponding to C13-114 was as shown in SEQ ID NO: 35.

The expression vectors C13-113-BsaI (SEQ ID NO: 36) and C13-114-BsaI (SEQ ID NO: 37) for C13-113 and C13-114 were synthesized by a reagent company.

According to the method described in Example 4, the fragments targeting the target sites were obtained by primer annealing means, wherein the primers used were as follows:

PTBP1-targeting

C13-113:
Forward Primer:
(SEQ ID NO: 38)
5′-CAACGTGGTTGGAGAACTGGATGTAGATGGGCTG-3′
Reverse Primer:
(SEQ ID NO: 23)
5′-AAAACAGCCCATCTACATCCAGTTCTCCAACCAC-3′
C13-114:
Forward Primer:
(SEQ ID NO: 39)
5′-atctGTGGTTGGAGAACTGGATGTAGATGGGCTG-3′
Reverse Primer:
(SEQ ID NO: 23)
5′-AAAACAGCCCATCTACATCCAGTTCTCCAACCAC-3′

According to the method described in Example 4, the synthesized plasmids C13-113-BsaI and C13-114-BsaI were digested with a Bsa I endonuclease, and linked to the annealed products with a T4 DNA ligase. After the transformation into Escherichia coli, the positive clones were selected and the plasmids were extracted. The 293T cells (293T cells from different batches used in Example 4) were subject to detection at 72 h after transfection. The blank control group was transfected with C13-2-BsaI in Example 4, respectively.

At 72 h after transfection, the cells were subject to RNA extraction, reverse transcription and qPCR (with the same primers used in Example 4) according to the method described in Example 4.

A reaction system was configured according to the instructions of the SYBR® Green Premix Pro Taq HS qPCR Kit, and detected by using a QuantStudio™ 5 Real-Time PCR System.

The amount of PTBP1 RNA was calculated by using a 2-44Ct method.

The calculation method was as follows:

$$Δ Ct = Ct ( PTBP 1 ) - Ct ( GAPDH )$$

$$ΔΔ Ct = Δ Ct ( a sample to be verified ) - Δ Ct ( C 13 - 2 - BsaI )$$

$${2}^{- Δ Δ C t} = 2 \hat{} ( - ΔΔ Ct ) .$$

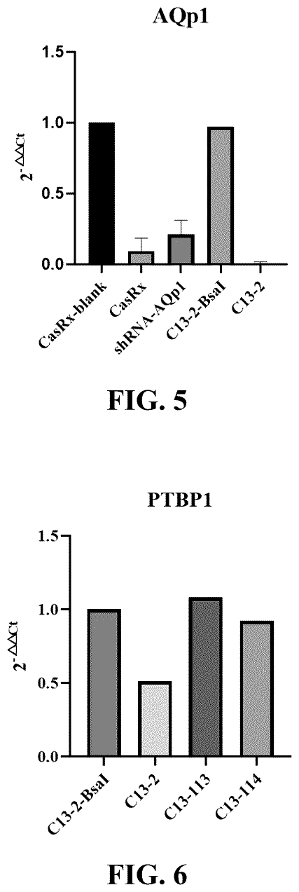

The experimental results were as shown in Table 6 and FIG. 6.

TABLE 6
Comparison of knockdown test results of PTBP1 RNA

| Group for experiment | PTBP1 RNA level |
|---|---|
| C13-2-BsaI | 1.00 |
| C13-2 | 0.51 |
| C13-113 | 1.08 |
| C13-114 | 0.92 |

The data in the table showed that in combination with the gRNA targeting PTBP1 of the present example, C13-2 was observed to have a relative high editing efficiency, which was better than that of C13-113 and C13-114.No obvious editing was observed in C13-113 group.

### Example 6: Off-Target Test of C13-2

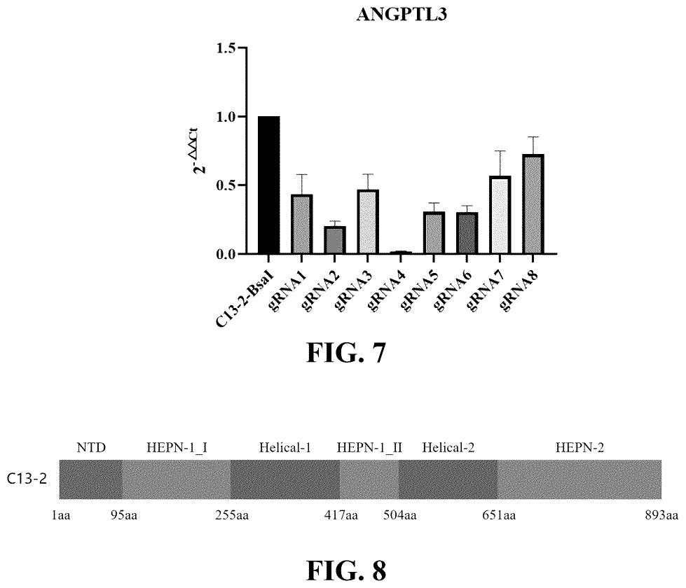

Prediction was performed in the whole genome and whole cDNA sequences of a target species (Homo sapiens) with EMBOSS-water program and NCBI-Blast program. Alignment was conducted with the sense and anti-sense strands of the guide sequence of a gRNA. The predicted results were filtered according to the difference of lengths of predicted target and guide sequence of gRNA was no more than four bases and the mismatch+gap was no more than four bases. The potential off-target information obtained was as shown in FIG. 7.

TABLE 7
Predicted number of potential off-target genes

| | | Potential Off- |
|---|---|---|
| | guide sequence | Target Gene No. |
| C13-2 gRNA/ | GUGGUUGGAGAACUG | 7 |
| CasRx gRNA | GAUGUAGAUGGGCUG | |
| | (SEQ ID NO: 6) | |
| shRNA1 | GCCCAUCUACAUCCA | 6700 |
| | GUUCUC | |
| | (SEQ ID NO: 40) | |
| shRNA2 | CAGCCCAUCUACAUC | 6882 |
| | CAGUUC | |
| | (SEQ ID NO: 41) | |

The plasmids of C13-2 targeting PTBP1, shRNA-PTBP1 (the shRNA expressed was named “shRNA2” in this example) and CasRx-blank in Example 4 were used.

The sequence of CasRx-PTBP1 plasmid (the positive control vector of CasRx targeting PTBP1) prepared by conventional method was as shown in SEQ ID NO: 8, and the structure of the plasmid was CMV-CasRx-U6-gRNA.

The plasmid expressing shRNAI was additionally constructed according to the method described in Example 4, and the difference to shRNA-PTBP1 plasmid was only the different guide sequence of encoded shRNA. The plasmid targeted PTBP1, too, and no additional g was added after U6 promoter.

293T cells were transfected with the aforementioned plasmids according to the method described in Example 4, respectively. The cells were subject to transfection in a 24-well plate according to the operation instructions of Lipofectamine™ 2000 (Thermo), and the RNA was extracted with a SteadyPure Universal RNA Extraction Kit AG21017.

RNA samples extracted were sequenced by PE150 bp RNA-Seq, and fastq files obtained by sequencing were aligned with the reference genome of the target species by HISAT2 or STAR software, to obtain BAM files after the alignment. The expression levels of the obtained transcripts and each gene were detected by kallisto, RSEM or HTSeq.

The variation analysis of expression levels among groups (variation relative to CasRx-blank group) was conducted by using DESeq2, limma-voom and edger, and a gene satisfying p.adj <0.05, |log 2FoldChange|>0.5 and basemean >2.5 was taken as the differential expression gene (DEG). The obtained DEG information was as follows:

TABLE 8
Number of DEGs and intersection with the predicted potential off-target gene

| | | | All&Sig | | | |
|---|---|---|---|---|---|---|
| | Up&Sig | Down&Sig | (number of up- | | | |
| | (number of up- | (number of down- | regulated or down- | Isec | Isec | Isec |
| Group | regulated DEG) | regulated DEG) | regulated DEG) | Up | Down | All |
| CasRx | 4 | 1 | 5 | 0 | 0 | 0 |
| shRNA1 | 182 | 337 | 519 | 45 | 117 | 162 |
| shRNA2 | 171 | 383 | 554 | 23 | 127 | 150 |
| C13-2 | 4 | 68 | 72 | 0 | 0 | 0 |

Note:
Up represents for up-regulation, and Down represents for down-regulation.

Sig represents for the significant difference in gene expression compared to control group (CaxRx-blank group).

Isec represents the number of DEG after taking intersection with the “potential off-target gene” predicted by the program.

It could be seen from the data in the table above that, the number of off-target sites of CasRx and C13-2 by transcriptome sequencing after taking intersection with predicted off-targets was 0, wherein there was hardly any off-target in C13-2, and there was a large number of off-target sites in shRNA1 and shRNA2. In terms of off-target safety, C13-2 had better performance than shRNA1 and shRNA2, and comparable to CasRx. Moreover, the size of C13-2 was only 893 aa, far less than that of CasRx (967 aa), and it was easier to be delivered with gRNA packaged into an AAV.

### Example 7: Editing Targeting ANGPTL3

This example was conducted according to the method described in Example 4.

The Lv-ANGPTL3-T2a-GFP vector (SEQ ID NO: 52) overexpressing ANGPTL3 gene and EGFP gene was constructed, wherein ANGPTL3 and EGFP were spaced apart by a 2A peptide. The Lv-ANGPTL3-T2a-GFP plasmid was packaged into a lentivirus and transduced into 293T cells to form a 293T cell line (namely 293T-ANGPTL3 cell) stably overexpressing ANGPTL3 gene.

A target site-targeted fragment was obtained by using a primer annealing manner.

After the Cas13-2-B sal vector (SEQ ID NO: 15) was digested with a BsaI endonuclease, the annealed products and the backbones purified and recovered after the digestion were subjected to linkage with a T4 DNA ligase. After the transformation into Escherichia coli, the positive clones were selected and the plasmid of C 13-2 targeting ANGPTL 3 (C13-2 protein expression was driven by CM V, gRNA expression was driven by U 6 promoter, the DR sequence of gRNA was as shown in SEQ ID NO: 3, and the guide sequence of gRNA was as shown in Table 9) was extracted and obtained. The plasmid was transfected with 293T-ANGPTL3 cells. The negative control group was transfected with the C13-2-Bsa I plasmid.

TABLE 9
Guide sequence of gRNA targeting ANGPTL3 RNA

| gRNA | gRNA guide sequence |
|---|---|
| ANGPTL3-gRNA1 | CAUGAAAAACUUGAGAGUUGCUGGGUCUGA |
| | (SEQ ID NO: 42) |
| ANGPTL3-gRNA2 | GAAUUAAGUUAGUUAGUUGCUCUUCUAAAU |
| | (SEQ ID NO: 43) |
| ANGPTL3-gRNA3 | CGAUGUUGAAUUAAUGUCCAUGGACUACCU |
| | (SEQ ID NO: 44) |
| ANGPTL3-gRNA4 | GAUAGAGAAAUUUCUGUGGGUUCUUGAAUA |
| | (SEQ ID NO: 45) |
| ANGPTL3-gRNA5 | CUGGAGAAGGUCUUUGAUGCUAUUAUCUUG |
| | (SEQ ID NO: 46) |
| ANGPTL3-gRNA6 | CACUAUGGAGUAUAUCUUCUCUAGGCCCAA |
| | (SEQ ID NO: 47) |
| ANGPTL3-gRNA7 | CCACACUCAUCAUGCCACCACCAGCCUCCU |
| | (SEQ ID NO: 48) |
| ANGPTL3-gRNA8 | GACCAUCUAAAAUUGAUUCCCACAUCACAA |
| | (SEQ ID NO: 49) |

The cells were subject to RNA extraction and reverse transcription at 72 h after transfection, and the reverse transcribed product was subject to qPCR detection.

Primers used in qPCR were as follows:

Detection of ANGPTL3:
Forward Primer:
(SEQ ID NO: 50)
5′-CCAGAACACCCAGAAGTAACT-3′
Reverse Primer:
(SEQ ID NO: 51)
5′-TCTGTGGGTTCTTGAATACTAGTC-3′
Detection of internal reference GAPDH:
Forward Primer:
(SEQ ID NO: 30)
5′-CCATGGGGAAGGTGAAGGTC-3′
Reverse Primer:
(SEQ ID NO: 31)
5′-GAAGGGGTCATTGATGGCAAC-3′

The amount of target RNA was calculated by using a relative quantitative method, i.e., 2^{-ΔΔCt} method.

The transfection of cells with recombinant plasmids, RNA extraction, reverse transcription and qPCR were all conducted independently in triplicate of biological experiments, and the average results of the triplicates were obtained. The results were as shown in Table 10 and FIG. 7.

TABLE 10
Knockdown test results of ANGPTL3 RNA

| Groups for | Experiment | Experiment | Experiment | |
|---|---|---|---|---|
| experiments | 1 | 2 | 3 | Average |
| C13-2-BsaI | 1.00 | 1.00 | 1.00 | 1.00 |
| ANGPTL3-gRNA1 | 0.54 | 0.27 | 0.49 | 0.43 |
| ANGPTL3-gRNA2 | 0.17 | 0.24 | 0.20 | 0.20 |
| ANGPTL3-gRNA3 | 0.59 | 0.38 | 0.44 | 0.47 |
| ANGPTL3-gRNA4 | 0.02 | 0.02 | 0.01 | 0.02 |
| ANGPTL3-gRNA5 | 0.26 | 0.28 | 0.38 | 0.31 |
| ANGPTL3-gRNA6 | 0.33 | 0.25 | 0.33 | 0.30 |
| ANGPTL3-gRNA7 | 0.73 | 0.37 | 0.60 | 0.57 |
| ANGPTL3-gRNA8 | 0.64 | 0.67 | 0.87 | 0.73 |

The experimental results showed that C 13-2 could achieve effective knockdown against ANGPTL 3 RNA, wherein gRNA 2, gRNA 4, gRNA5 and gRNA 6 had significant editing effect.

### Example 8: Editing with the Dead Mutant of C 13-2 (dC 13-2)

C13-2-VEGFA vector (SEQ ID NO: 72) was constructed for expression of C13-2 protein and gRNA targeting VEGFA. The guide sequence of this gRNA was

(SEQ ID NO: 73)
TGGGTGCAGCCTGGGACCACTTGGCATGG.

Then, R4xH mutant verified vectors of C13-2 were constructed from C13-2-VEGFA vector with conventional homologous recombination method, and were as shown in Table 11.

The mutant vector was introduced with the following mutations comparing to the coding sequence of C13-2 in the C13-2-VEGFA vector:

R210A + H215A:
AGAAACGCCACCGCCCAC (SEQ ID NO: 74) →
GCAAACGCCACCGCCGCC (SEQ ID NO: 75);
R750A + H755A:
AGAAAGACCAAGAGACAC (SEQ ID NO: 76) →
GCAAAGACCAAGAGAGCC (SEQ ID NO: 77);
and/or
R785A + H790A:
AGAAACGACGTGGAGCAC (SEQ ID NO: 78) →
GCAAACGACGTGGAGGCC (SEQ ID NO: 79).

TABLE 11
Vectors used for testing dead mutants of C13-2

| Plasmid vector | Notes |
|---|---|
| C13-2-BsaI | Negative control of C13-2 |
| (SEQ ID NO: 15) | |
| C13-2-VEGFA | Expressing wild-type C13-2 |
| (SEQ ID NO: 72) | |
| R4xH-1-VEGFA | Expressing the C13-2 mutant (R210A/H215A), |
| | with the first R4xH of C13-2 mutated |
| R4xH-2-VEGFA | Expressing the C13-2 mutant (R750A/H755A), |
| | with the second R4xH of C13-2 mutated |
| R4xH-3-VEGFA | Expressing the C13-2 mutant (R785A/H790A), |
| | with the third R4xH of C13-2 mutated |
| R4xH(1, 3)- | Expressing the C13-2 mutant (R210A/H215A, |
| VEGFA | R785A/H790A), with the first and the third |
| | R4xH of C13-2 mutated |
| R4xH(1, 2, 3)- | Expressing the C13-2 mutant (R210A/H215A, |
| VEGFA | R750A/H755A, R785A/H790A), with all the |
| | three R4xHs of C13-2 mutated |
| 293T-NC | Untreated 293T cells |

The vectors were transfected with the 293T cell line. The transfection was operated according to the instructions of Lipofectamine™ 2000 (Thermo). The RNA was extracted with SteadyPure Universal RNA Extraction K it at 72 h after transfection. The extracted RNA of three batches were sent to the sequencing company for RNA seq sequencing, and the detected amount of VEGFA RNA was as shown in Table 12 below:

TABLE 12
The amount of VEGFA RNA determined by RNAseq sequencing

| | | The reduced amount |
|---|---|---|
| | TPM value | compared to |
| Group | (average) | C13-2-BsaI group |
| C13-2-BsaI | 67.99067264 | 0 |
| R4xH-1-VEGFA | 61.02221547 | 10.25 |
| R4xH-2-VEGFA | 36.83780119 | 45.82 |
| R4xH-3-VEGFA | 57.89735168 | 14.85 |
| R4xH(1, 3)-VEGFA | 52.12446721 | 23.34 |
| R4xH(1, 2, 3)-VEGFA | 64.72357693 | 4.81 |

The experimental data in Table 12 showed that the editing activity was still high after introduction of R 750A+H 755A mutation, weak editing activity was retained after introduction of R210A+H 215A and/or R 785A+H 790A mutation, and the editing activity was almost lost completely only after introduction of R210A+H 215A, R 750A+H 755A and R 785A+H 790A mutations at the same time.

### Example 9: Test of C 13-2 Truncate

### Construction of the Verified Vector of C 13-2 Truncate Targeting Endogenous Gene VEGFA

The verified vectors of truncates shown in Table 13 were constructed from the C13-2-VEGFA plasmid (SEQ ID NO: 72) by using the three-fragment homologous recombination method, and the only difference from the C13-2-VEGFA vector was that the coding sequence of C13-2 was truncated. The vectors could express each truncated proteins and the gR NA targeting VEGFA.

TABLE 13
Constructed verified vector of C13-2 truncates

| Vector | Expression product/Notes |
|---|---|
| C13-2-BsaI | Negative control |
| (SEQ ID NO: 15) | |
| C13-2-VEGFA | Wild-type C13-2 |
| C10 | Truncate with deletion of amino acids at positions 91-120 of C13-2 |
| C16 | Truncate with deletion of amino acids at positions 141-180 of C13-2 |
| C22 | Truncate with deletion of amino acids at positions 211-240 of C13-2 |
| C34 | Truncate with deletion of amino acids at positions 331-360 of C13-2 |
| C38 | Truncate with deletion of amino acids at positions 351-400 of C13-2 |
| C44 | Truncate with deletion of amino acids at positions 431-460 of C13-2 |
| C48 | Truncate with deletion of amino acids at positions 461-500 of C13-2 |
| C54 | Truncate with deletion of amino acids at positions 511-550 of C13-2 |
| C62 | Truncate with deletion of amino acids at positions 611-640 of C13-2 |
| C64 | Truncate with deletion of amino acids at positions 631-660 of C13-2 |
| C67 | Truncate with deletion of amino acids at positions 661-690 of C13-2 |
| C74 | Truncate with deletion of amino acids at positions 691-760 of C13-2 |
| C84 | Truncate with deletion of amino acids at positions 821-860 of C13-2 |
| C87 | Truncate with deletion of amino acids at positions 861-890 of C13-2 |
| 293T-NC | Control group, untransfected 293T cells |

The verified vectors and control vectors were transfected into the 293T cell line according to the operation instructions of Lipofectamine™ 2000 (Thermo). The RNA was extracted with SteadyPure Universal RNA Extraction Kit at 72 h after transfection, and was the subject to reverse transcription by using the Evo M-MLV Mix Kit with gDNA Clean for qPCR reverse transcription kit. A reaction system was configured according to the instructions of the SY BR® Green Premix Pro Taq HS qPCR Kit, and detected by using a QuantStudio™ 5 Real-Time PCR System.

The primers used in the qPCR were as follows:

Detection of VEGFA:
(SEQ ID NO: 88)
ACCTCCACCATGCCAAGTGG
(SEQ ID NO: 89)
CAGGGTCTCGATTGGATGGC
Detection of internal reference GAPDH:
(SEQ ID NO: 30)
CCATGGGGAAGGTGAAGGTC
(SEQ ID NO: 31)
GAAGGGGTCATTGATGGCAAC

The target RNA was calculated by using a relative quantitative method, i.e., 2-44Ct method. Multiple repeated experiments were conducted, and the average results were taken. The results were as shown in Table 14.

TABLE 14
The relative amount of VEGFA RNA after editing with truncates

| Group | Relative amount of VEGFA RNA |
|---|---|
| C13-2-BsaI | 1.00 |
| C13-2-VEGFA | 0.06 |
| C10 | 0.68 |
| C16 | 0.52 |
| C22 | 0.51 |
| C34 | 0.77 |
| C38 | 0.51 |
| C44 | 0.65 |
| C48 | 0.66 |
| C54 | 0.72 |
| C62 | 0.62 |
| C64 | 0.69 |
| C67 | 0.69 |
| C74 | 0.65 |
| C84 | 0.81 |
| C87 | 0.90 |
| 293T-NC | 1.05 |

The C13-2 truncates in the present example retained a certain intensity of RNA-editing activity.

### Example 10: Test for Different DR (Direct Repeated) Sequences

Verified vectors (as shown in Table 16) targeting endogenous genes VEGFA and PTBP1 were constructed, and encoding different DR sequences (as shown in Table 15).

TABLE 15
Different DR sequences designed

| DR | | |
|---|---|---|
| name | DR sequence | Notes |
| DRrc | GGAAGATAACTCTACAAACCTGTAGG | — |
| | GTTCTGAGAC (SEQ ID NO: 3) | |
| DR2 | CCGCACAGTCCCTACAGGTTTGTAGA | — |
| | GTCATCTTCC (SEQ ID NO: 80) | |
| DR2rc | GGAAGATGACTCTACAAACCTGTAGG | Reverse |
| | GACTGTGCGG (SEQ ID NO: 81) | complementary |
| | | to DR2 |
| DR3 | GGTGTACAGGGTGCCTGGATTTGACA | — |
| | GGGTTACAGC (SEQ ID NO: 82) | |
| DR3rc | GCTGTAACCCTGTCAAATCCAGGCAC | Reverse |
| | CCTGTACACC (SEQ ID NO: 83) | complementary |
| | | to DR3 |
| DR4 | GGTGTACAGGGTGCCTAGATTTGACA | — |
| | GGGTTACAGC (SEQ ID NO: 84) | |
| DR4rc | GCTGTAACCCTGTCAAATCTAGGCAC | Reverse |
| | CCTGTACACC (SEQ ID NO: 85) | complementary |
| | | to DR4 |
| DR- | GGAAGAACTCTACAAACCTGTAGGGT | Unmatched 2 nt |
| hf1 | TCTGAGAC (SEQ ID NO: 86) | in DRrc stem |
| | | region deleted |
| DR- | GGAAGATAACTCTACAAACCTGTAGA | U-G base pair |
| hf2 | GTTCTGAGAC (SEQ ID NO: 87) | in DRrc stem |
| | | region changed |
| | | to U-A |

TABLE 16
Vector plasmids constructed for expression of various gRNA

| | Target RNA | |
|---|---|---|
| | of the gRNA | |
| | expressed by | |
| Plasmid Name | each plasmid | Notes |
| C13-2-BsaI | Not targeting | Not targeting |
| | VEGFA or | VEGFA or PTBP1 |
| | PTBP1 | |
| C13-2-VEGFA | VEGFA | The DR sequence is DRrc. |
| C13-2-DR2-VEGFA | VEGFA | The DR sequence is DR2. |
| C13-2-DR2rc-VEGFA | VEGFA | The DR sequence is DR2rc. |
| C13-2-DR3-VEGFA | VEGFA | The DR sequence is DR3. |
| C13-2-DR3rc-VEGFA | VEGFA | The DR sequence is DR3rc. |
| C13-2-DR4-VEGFA | VEGFA | The DR sequence is DR4. |
| C13-2-DR4rc-VEGFA | VEGFA | The DR sequence is DR4rc. |
| C13-2-DR-hf1-VEGFA | VEGFA | The DR sequence is DR-hf1. |
| C13-2-DR-hf2-VEGFA | VEGFA | The DR sequence is DR-hf2. |
| C13-2-PTBP1 | PTBP1 | The DR sequence is DRrc. |
| C13-2-DR2-PTBP1 | PTBP1 | The DR sequence is DR2. |
| C13-2-DR2rc-PTBP1 | PTBP1 | The DR sequence is DR2rc. |
| C13-2-DR3-PTBP1 | PTBP1 | The DR sequence is DR3. |
| C13-2-DR3rc-PTBP1 | PTBP1 | The DR sequence is DR3rc. |
| C13-2-DR4-PTBP1 | PTBP1 | The DR sequence is DR4. |
| C13-2-DR4rc-PTBP1 | PTBP1 | The DR sequence is DR4rc. |
| C13-2-DR-hf1-PTBP1 | PTBP1 | The DR sequence is DR-hf1. |
| C13-2-DR-hf2-PTBP1 | PTBP1 | The DR sequence is DR-hf2. |
| 293T-NC | N/A | Untreated 293T cells |

The verified vectors expressing various different crRNA sequences (5′-guide-DR-3′) in Table 16 were constructed with conventional means (only substitution of the expression frame sequence of crRNA occurred) from the C13-2-VEGFA vectors in Example 8 and the vector plasmid of C13-2 targeting PTBP1 in Example 4.

The 293T cells were transfected with the verified vectors and control vectors according to the method described in the above examples. The cells were subject to RNA extraction, reverse transcription and detection with a qPCR kit after 72 h. Primers used in the qPCR were as follows:

Detection of VEGFA:
(SEQ ID NO: 88)
ACCTCCACCATGCCAAGTGG
(SEQ ID NO: 89)
CAGGGTCTCGATTGGATGGC
Detection of PTBP1:
(SEQ ID NO: 26)
ATTGTCCCAGATATAGCCGTTG
(SEQ ID NO: 27)
GCTGTCATTTCCGTTTGCTG
Detection of internal reference GAPDH:
(SEQ ID NO: 30)
CCATGGGGAAGGTGAAGGTC
(SEQ ID NO: 31)
GAAGGGGTCATTGATGGCAAC

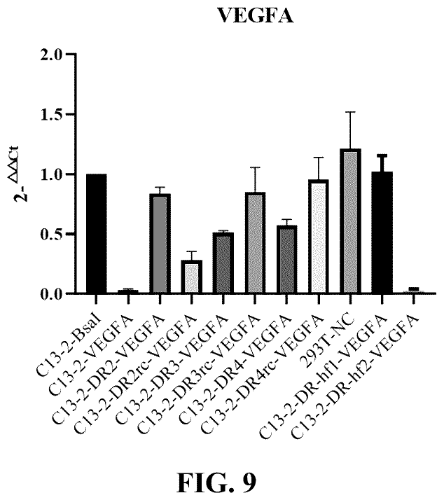

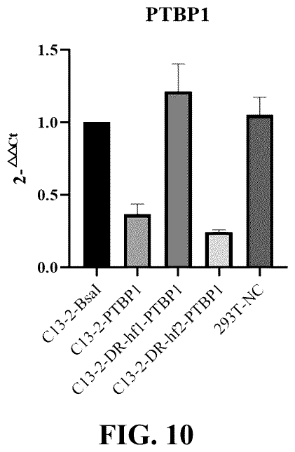

The change of target RNA was calculated by using the 2-44Ct method. Multiple repeated experiments were conducted, and the average results were taken. The results were as shown in Table 17, Table 18, FIG. 9 and FIG. 10.

TABLE 17
Relative amount of VEGFA RNA after
editing with different gRNA.

| | Relative amount of |
|---|---|
| VEGFA | VEGFA RNA |
| C13-2-BsaI | 1.00 |
| C13-2-VEGFA | 0.03 |
| C13-2-DR2-VEGFA | 0.84 |
| C13-2-DR2rc-VEGFA | 0.28 |
| C13-2-DR3-VEGFA | 0.51 |
| C13-2-DR3rc-VEGFA | 0.85 |
| C13-2-DR4-VEGFA | 0.57 |
| C13-2-DR4rc-VEGFA | 0.95 |
| C13-2-DR-hf1-VEGFA | 1.02 |
| C13-2-DR-hf2-VEGFA | 0.03 |
| 293T-NC | 1.22 |

TABLE 18
Relative amount of PTBP1 RNA after editing with different gRNA

| PTBP1 | Average |
|---|---|
| C13-2-BsaI | 1.00 |
| C13-2-PTBP1 | 0.37 |
| C13-2-DR-hf1-PTBP1 | 1.21 |
| C13-2-DR-hf2-PTBP1 | 0.25 |
| 293T-NC | 1.05 |

The experimental results in Table 17 and Table 18 showed that the editing efficiency was the highest when DRrc or DR-hf2 were used, which was better than that with other DR sequences (P<0.05). When DR2rc, DR3 or DR4 was used, a relative high editing efficiency could be achieved, which was better than that with DR2, DR3rc, DR4rc or DR-hf1.

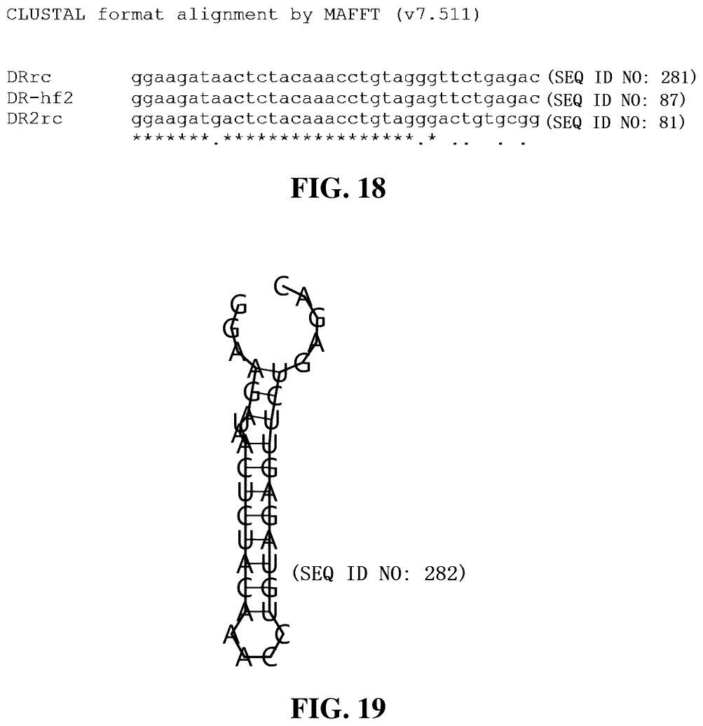

The sequence alignment results of direct repeated sequences DRrc, DR-hf2 and DR2rc were as shown in FIG. 18.

The RNA secondary structure of direct repeated sequence DR-hf2 predicted by RNAfold was shown in FIG. 19.

### Example 11: Comparison of C13-2 with the Main Stream of Cas 13 Tools

All verified vectors and control vectors (Table 19) in this example utilized the same scaffold sequence with that in C13-2-BsaI (SEQ ID NO: 15), and the only difference was the coding sequence of Cas13 and crRNA.

The NLS sequences and the linking sequences thereof of each vector were the same.

The structure of all vectors was CMV-NLS-Cas13-2xNLS-U6-crRNA, and all Cas13 carried one NLS at N-terminus and two NLSs at C-terminus with the structure of 3×NLS in total.

As EGFP sequence was absent in 293T cells, the vector targeting GFP was used for negative control.

The guide sequence targeting GFP:
(SEQ ID NO: 90)
tgccgttcttctgcttgtcggccatgatat.
The guide sequence targeting PTBP1:
(SEQ ID NO: 6)
GTGGTTGGAGAACTGGATGTAGATGGGCTG
The guide sequence targeting VEGFA:
(SEQ ID NO: 73)
TGGGTGCAGCCTGGGACCACTTGGCATGG.

TABLE 19
Verified vectors and control vectors constructed for
comparison with the main stream of Cas13 tools

| Plasmid Name | Structure |
|---|---|
| CasRx-3NLS-GFP | CMV-NLS-CasRx-2xNLS-U6-DR-GFP guide |
| CasRx-3NLS-PTBP1 | CMV-NLS-CasRx-2xNLS-U6-DR-PTBP1 guide(SEQ ID NO: 91) |
| PspCas13b-3NLS-GFP | CMV-NLS-PspCas13b-2xNLS-U6-GFP guide-DR |
| PspCas13b-3NLS-PTBP1 | CMV-NLS-PspCas13b-2xNLS-U6-PTBP1 guide-DR(SEQ ID NO: 92) |
| PspCas13b-3NLS-VEGFA | CMV-NLS-PspCas13b-2xNLS-U6-VEGFA guide-DR |
| Cas13X.1-3NLS-GFP | CMV-NLS-Cas13X.1-2xNLS-U6-GFP guide-DR |
| Cas13X.1-3NLS-PTBP1 | CMV-NLS-Cas13X.1-2xNLS-U6-PTBP1 guide-DR(SEQ ID NO: 93) |
| Cas13X.1-3NLS-VEGFA | CMV-NLS-Cas13X.1-2xNLS-U6-VEGFA guide-DR |
| Cas13Y.1-3NLS-GFP | CMV-NLS-Cas13Y.1-2xNLS-U6-GFP guide-DR |
| Cas13Y.1-3NLS-PTBP1 | CMV-NLS-Cas13Y.1-2xNLS-U6-PTBP1 guide-DR(SEQ ID NO: 94) |
| Cas13Y.1-3NLS-VEGFA | CMV-NLS-Cas13Y.1-2xNLS-U6-VEGFA guide-DR |
| C13-2-GFP | CMV-NLS-C13-2-2xNLS-U6-DR-GFP guide |
| C13-2-PTBP1 | CMV-NLS-C13-2-2xNLS-U6-DR-PTBP1 guide |
| C13-2-VEGFA | CMV-NLS-C13-2-2xNLS-U6-DR-VEGFA guide |

To save space, the sequences of four vectors (SEQ ID NOs: 91-94) in Table 19 were shown exemplary.

The293T cells were transfected with the verified vectors and control vectors. Untransfected plasmids were used as blank control.

The transfection was performed according to the operation instructions of Lipofectamine™ 2000 (Thermo). At 48 h after transfection, the cells were subjected to RNA extraction with a SteadyPure Universal RNA Extraction Kit, and the RNA concentration was detected with an ultramicro spectrophotometer. The RNA product was subject to reverse transcription by using the Evo M-MLV Mix Kit with gDNA Clean for qPCR reverse transcription kit. A reaction system was configured according to the instructions of the SYBR® Green Premix Pro Taq HS qPCR Kit, and detected by using a QuantStudio™ 5 Real-Time PCR System.

Primers used in the qPCR were as follows:

Detection of VEGFA:
(SEQ ID NO: 88)
ACCTCCACCATGCCAAGTGG
(SEQ ID NO: 89)
CAGGGTCTCGATTGGATGGC
Detection of PTBP1:
(SEQ ID NO: 26)
ATTGTCCCAGATATAGCCGTTG
(SEQ ID NO: 27)
GCTGTCATTTCCGTTTGCTG
Detection of internal reference GAPDH:
(SEQ ID NO: 30)
CCATGGGGAAGGTGAAGGTC
(SEQ ID NO: 31)
GAAGGGGTCATTGATGGCAAC

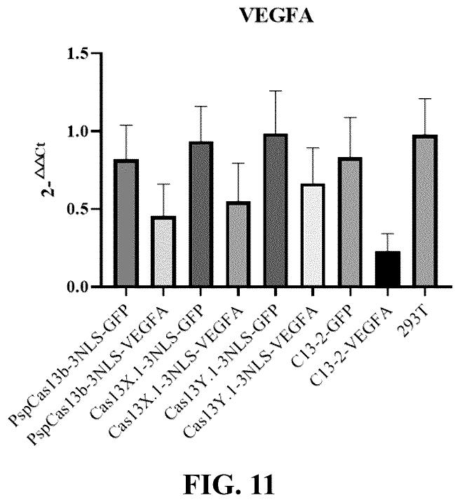

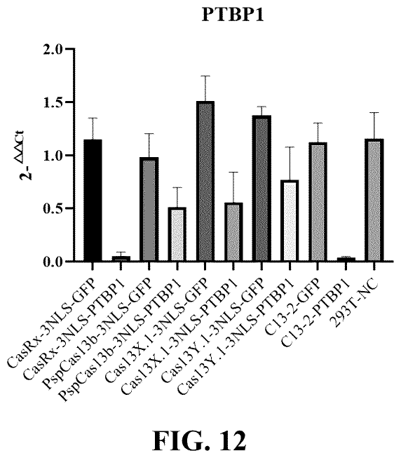

The amount of target RNA was calculated by using the 2-44Ct method, and each Cas13 protein took corresponding GFP-targeting group as negative control. Multiple repeated experiments were conducted, and the average results were taken. The results were as shown in Table 20, Table 21, FIG. 11 and FIG. 12.

TABLE 20
C13-2 and known Cas13 tool targeting VEGFA RNA

| | Relative amount of VEGFA |
|---|---|
| Group | RNA after editing |
| PspCas13b-3NLS-GFP | 0.82 |
| PspCas13b-3NLS-VEGFA | 0.45 |
| Cas13X.1-3NLS-GFP | 0.94 |
| Cas13X.1-3NLS-VEGFA | 0.55 |
| Cas13Y.1-3NLS-GFP | 0.98 |
| Cas13Y.1-3NLS-VEGFA | 0.67 |
| C13-2-GFP | 0.83 |
| C13-2-VEGFA | 0.23 |
| 293T blank control | 0.98 |

In comparison of editing effects of VEGFA target, C13-2 had excellent editing effect, which was better than the current main stream of Cas13 editing tools. The editing efficiency was C13-2>PspCas13b>Cas13X.1>Cas13Y.1.

TABLE 21
C13-2 and known Cas13 tool targeting PTBP1 RNA

| | Relative amount of PTBP1 |
|---|---|
| Group | RNA after editing |
| CasRx-3NLS-GFP | 1.15 |
| CasRx-3NLS-PTBP1 | 0.05 |
| PspCas13b-3NLS-GFP | 0.98 |
| PspCas13b-3NLS-PTBP1 | 0.51 |
| Cas13X.1-3NLS-GFP | 1.51 |
| Cas13X.1-3NLS-PTBP1 | 0.56 |
| Cas13Y.1-3NLS-GFP | 1.37 |
| Cas13Y.1-3NLS-PTBP1 | 0.77 |
| C13-2-GFP | 1.13 |
| C13-2-PTBP1 | 0.03 |
| 293T blank control | 1.16 |

In comparison of editing effects of PTBP1 target, C13-2 had excellent editing effect and the value of editing efficiency was C13-2>CasRx>PspCas13b>Cas13X.1>Cas13Y.1. The editing efficiency of C13-2 was significantly superior to PspCas13b, Cas13X.1 and Cas13Y.1 (P<0.05).

### Example 12: Construction of Single-Base Editor from dC13-2

The target used in this example to verify the single-base editing effect was EGFP, and the guide sequence for targeting EGFP was tgccgttcttctgcttgtcggccatgatatagacgttgtggctgttgtagttgtactccagcttgtgccc (SEQ ID NO: 95).

The vectors involving dC13-2 all expressed the dead mutant of C13-2 comprising R210A+H215A, R750A+H755A and R785A+H790A mutations.

TABLE 22
Plasmid vectors and instructions related to single base editing

| Plasmid Name | Notes |
|---|---|
| dC13-2-BsaI(SEQ ID NO: 96) | All three R4XHs of C13-2 were mutated |
| dC13-2-EGFP(SEQ ID NO: 97) | No ADAR was used, as negative control |
| dC13-2-ADAR-EGFP (SEQ ID | ADAR was used, no linking peptide was |
| NO: 98) | used |
| dC13-2-A(EAAAK)3A-ADAR-EGFP | ADAR was used, rigid linking peptide |
| (SEQ ID NO: 99) | A(EAAAK)₃A was used |
| dC13-2-(GGGGS)3-ADAR-EGFP | ADAR was used, flexible linking peptide |
| (SEQ ID NO: 100) | (GGGGS)₃ was used |
| pAAV-CMV-EGFP (SEQ ID | Plasmid expressing EGFP |
| NO: 101) | |
| PLKO-PURO-PspGRNA-EGFP | Expressing the gRNA used in combination |
| (SEQ ID NO: 102) | with PspCas13b |
| pC0055-CMV-dPspCas13b-GS- | Commercial plasmid expressing |
| ADAR2DD (addgene) | dPspCas13b-ADAR |

Vectors dC13-2-BsaI and dC13-2-EGFP were constructed according to conventional method.

The verified vector of single-base editing dC13-2-ADAR-EGFP, dC13-2-A (EAAAK) 3A-ADAR-EGFP and dC13-2-(GGGGS) 3-ADAR-EGFP were obtained by homologous recombination from dC13-2-BsaI plasmid, dC13-2-EGFP plasmid and pC0055-CMV-dPspCas13b-GS-ADAR2DD plasmid.

The pC0055-CMV-dPspCas13b-GS-ADAR2DD was taken as positive control. As the plasmid did not comprise a gRNA expression frame, the gRNA expression vector PLKO—PURO-PspGRNA-EGFP was synthesized by an outsourced company.

The transfection was operated according to the instructions of Lipofectamine™ 2000 (Thermo), and the vector to be verified and the EGFP reporter vector pAAV-CMV-EGFP were subject to transfection at 4:1. The transfection protocol was as shown in Table 23 below, wherein pC0055-CMV-dPspCas13b-GS-ADAR2DD and PLKO—PURO-PspGRNA-EGFP-AD were subject to co-transfection with each of 200 ng as they expressed PspCas13b-ADAR protein and gRNA respectively.

TABLE 23
The transfection protocol of the verified
vector and reporter vector for single-base editing

| Cell | 293T | |
|---|---|---|
| | | Reporter |
| Vector ratio | Verified vector | vector |
| Transfected | dC13-2-EGFP | pAAV-CMV- |
| Verified vector: | dC13-2-ADAR-EGFP | EGFP |
| Reporter vector = | dC13-2-A(EAAAK) | |
| 4:1(400 g::100 ng) | 3A-ADAR-EGFP | |
| | dC13-2-(GGGGS)3- | |
| | ADAR-EGFP | |
| Verified vector: | pC0055-CMV- | |
| Reporter vector = | dPspCas13b- | |
| 2:2:1(200:200:100 | GS-ADAR2DD | |
| ng) | PLKO-PURO- | |
| | PspGRNA-EGFP | |

At 48 h after transfection, the cells were subjected to RNA extraction with a SteadyPure Universal RNA Extraction Kit, and the RNA concentration was detected with an ultramicro spectrophotometer. The RNA product was subject to reverse transcription by using the Evo M-MLV RT-PCR Universal Transcriptase Kit. The transcription product was subject to PCR with identified primers, and the PCR product was sent to a sequencing company for sequencing.

Sequences of the identified primers were as follows (the length of product was 704 bp):

(SEQ ID NO: 103)
agggcgaggagctgtt,
(SEQ ID NO: 104)
gtacagctcgtccatgccg.

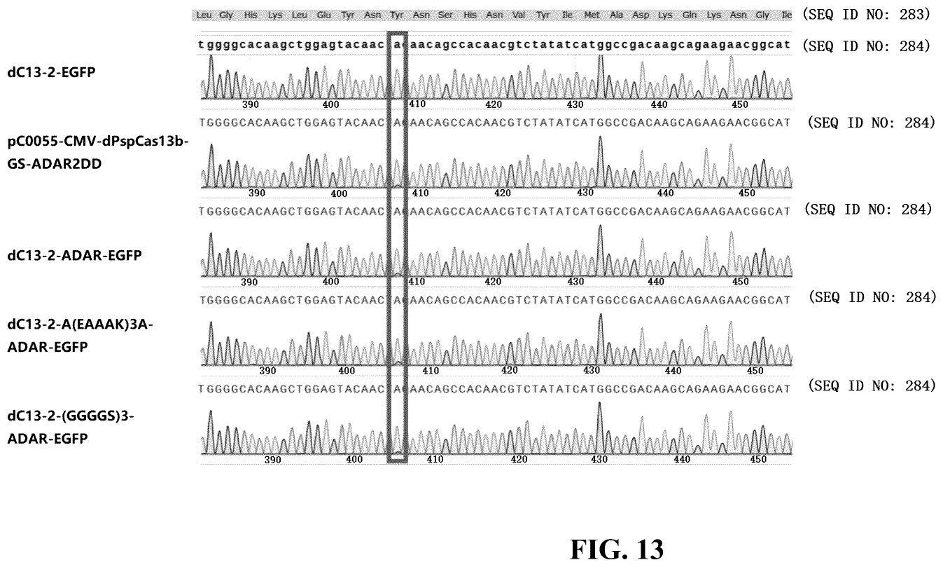

The sequencing results were as shown in FIG. 13, wherein an A->G conversion was occurred at the corresponding position in the target RNA to the base at position 48 of the guide sequence, indicating that the dC13-2 editor constructed by the inventor(s) induced single-base editing successfully.

There was no base conversion occurred in negative control dC13-2-EGFP group, and base conversion was induced to occur in positive control dPspCas13b-ADAR group. The base conversion was induced to occur when no linking peptide, rigid linking peptide A (EAAAK) 3A or flexible linking peptide (GGGGS) 3 used between dC13-2 and ADAR.

### Example 13: First Round of Mutation of C13-2

The principle of the first round of mutation: Using the structure of C13-2 predicted by AlphaFold v2.1, N->A mutation and R->A mutation were made on aa 1-89 and aa 263-417 belonging to REC lobe.

The designed mutants were as shown in Table 24 below.

TABLE 24
The first round of mutation

| Mutant Name | Mutation Site |
|---|---|
| M01 | R11A |
| M02 | N34A |
| M03 | R35A |
| M04 | R47A |
| M05 | R58A |
| M06 | R63A |
| M07 | R64A |
| M08 | N68A |
| M09 | N87A |
| M10 | N265A |
| M11 | N274A |
| M12 | R276A |
| M13 | R290A |
| M14 | R294A |
| M15 | N299A |
| M16 | N303A |
| M17 | R308A |
| M18 | R314A |
| M19 | R320A |
| M20 | R328A |
| M21 | N332A |
| M22 | R341A |
| M23 | N346A |
| M24 | R358A |
| M25 | N372A |
| M26 | N383A |
| M27 | N390A |
| M28 | N394A |

### Construction of Verified Vector

The first round of mutation was verified with target VEGFA. Using the C13-2-VEGFA vector in the previous examples as the encoding vector of wild-type C13-2, modification was made with Site Mutation Kit Mut Express II Fast Mutagenesis Kit V2 from Vazyme to obtain the expression constructs (verified vectors) for each mutant, which were used to express the C13-2 mutant and the gRNA targeting VEGFA. Primers used were as shown in Table 25 below.

TABLE 25
Primer sequences for verified vectors

| | | SEQ |
|---|---|---|
| | | ID |
| Mutant | Primer Sequence | NO |
| M01 | AACCAAGGCCAAGgcAATGGGCGTGAAGGCCCT | 105 |
| | ATTgcCTTGGCCTTGGTTTTCTTGTCCTTGCTC | 106 |
| M02 | CgcCAGAAGCAAGATCGAGTTCACCGAGGGCTA | 107 |
| | TCGATCTTGCTTCTGgcGCCCTTGCCGAAGGTGGT | 108 |
| M03 | CAACgcAAGCAAGATCGAGTTCACCGAGGGCTA | 109 |
| | TCGATCTTGCTTgcGTTGCCCTTGCCGAAGGTG | 110 |
| M04 | TACCACGGCgcAGCCCTGGAGACACCCAAGCAC | 111 |
| | CAGGGCTgcGCCGTGGTAGCCCTCGGTGAACTC | 112 |
| M05 | AAGCACTTCGGCATCgcAGGCTTCGAGGTGAGA | 113 |
| | AGAATCG | |
| | TgcGATGCCGAAGTGCTTGGGTGTCTCCAGGGC | 114 |
| M06 | TTCGAGGTGAgcAGAATCGACGAGAACGTGGACC | 115 |
| | GATTCTgcTCACCTCGAAGCCTCTGATGCCGAA | 116 |
| M07 | AGGTGAGAAgcAATCGACGAGAACGTGGACCTG | 117 |
| | GTCGATTgcTTCTCACCTCGAAGCCTCTGATGC | 118 |
| M08 | AAGAATCGACGAGgcCGTGGACCTGTGCGGCGA | 119 |
| | ACGgcCTCGTCGATTCTTCTCACCTCGAAGCCT | 120 |
| M09 | TGGTGgcCCCCAGCGAGAAGGTGGGCGAGGACT | 121 |
| | TCTCGCTGGGGgcCACCAGCAGGGCCTCGATGG | 122 |
| M10 | AAGAACGCCGTGgcCATGGCCATCCTGTTCGACC | 123 |
| | CATGgcCACGGCGTTCTTGCTCAGGAAGTCCTT | 124 |
| M11 | TGTTCGACCTGCTGgcCGCCAGAGACGTGGAGCA | 125 |
| | CGgcCAGCAGGTCGAACAGGATGGCCATGTTCA | 126 |
| M12 | CgcAGACGTGGAGCAGAAGAAGCAGATCACCGA | 127 |
| | TTCTGCTCCACGTCTgcGGCGTTCAGCAGGTCGA | 128 |
| | AC | |
| M13 | GAGTTCTACgcATTCACCATCAGAAAGGACGGC | 129 |
| | GGTGAATgcGTAGAACTCGTCGGTGATCTGCTT | 130 |
| M14 | TTCACCATCgcAAAGGACGGCAAGAACCTGGGC | 131 |
| | GTCCTTTgcGATGGTGAATCTGTAGAACTCGTCG | 132 |
| M15 | CAAGgcCCTGGGCATGAACCTGGTGAAGATCAG | 133 |
| | TTCATGCCCAGGgcCTTGCCGTCCTTTCTGATGG | 134 |
| M16 | CATGgcCCTGGTGAAGATCAGAGAGATCATCAT | 135 |
| | ATCTTCACCAGGgcCATGCCCAGGTTCTTGCCG | 136 |
| M17 | GTGAAGATCgcAGAGATCATCATCGACAGATACGC | 137 |
| | GATCTCTgcGATCTTCACCAGGTTCATGCCCAG | 138 |
| M18 | TCATCATCGACgcATACGCCAGCGGCCTGAGAG | 139 |
| | CGTATgcGTCGATGATGATCTCTCTGATCTTCA | 140 |
| M19 | CCTGgcAGACAAGAAGCACGACCCCCACAGACA | 141 |
| | TGCTTCTTGTCTgcCAGGCCGCTGGCGTATCTG | 142 |
| M20 | CCACgcACAGAAGATCAACGTGATCGCCGACTT | 143 |
| | TTGATCTTCTGTgcGTGGGGGTCGTGCTTCTTG | 144 |
| M21 | ACAGAAGATCgcCGTGATCGCCGACTTCCTGAT | 145 |
| | ATCACGgcGATCTTCTGTCTGTGGGGGTCGTGC | 146 |
| M22 | ACTTCCTGATCTTCgcAGCCCTGAGCCAGAACCA | 147 |
| | GG | |
| | CTgcGAAGATCAGGAAGTCGGCGATCACGTTG | 148 |
| M23 | CCAGgcCCAGGGCATCATCGACAAGACCGTGAG | 149 |
| | ATGATGCCCTGGgcCTGGCTCAGGGCTCTGAAGA | 150 |
| M24 | CAGCCTGgcACTGACCAAGGACGAGGAGGAGAA | 151 |
| | TTGGTCAGTgcCAGGCTGCTCACGGTCTTGTCG | 152 |
| M25 | ACCACGTGTACCAGgcCGCCGCCGAGCTGGTGTG | 153 |
| | CGgcCTGGTACACGTGGTCCTTCTCCTCCTCGT | 154 |
| M26 | TGGTGAGCgcCTGCCTGACCCCCTACTTCAACG | 155 |
| | TCAGGCAGgcGCTCACCATGCCCCACACCAGCT | 156 |
| M27 | CTACTTCgcCGACCCCAAGAACAAGTACATCCT | 157 |
| | TTGGGGTCGgcGAAGTAGGGGGTCAGGCAGTTG | 158 |
| M28 | CAAGgcCAAGTACATCCTGAAGTACAAGGACGC | 159 |
| | AGGATGTACTTGgcCTTGGGGTCGTTGAAGTAGGG | 160 |

### Transfection

The 293T cells were transfected with verified vectors and control vectors according to the instructions of Lipofectamine™ 2000 (Thermo). The C13-2-BsaI control group was transfected with the C13-2-BsaI vector in previous examples, the WT control group was transfected with the C13-2-VEGFA vector, both of which expressed wild-type C13-2. A 293T cell control group was set additionally, without transfection with any plasmid.

Detection of RNA Level with qPCR

At 48 h after transfection, the cells were subjected to RNA extraction with a SteadyPure Universal RNA Extraction Kit, and the RNA concentration was detected with an ultramicro spectrophotometer. The RNA product was subject to reverse transcription by using the Evo M-MLV Mix Kit with gDNA Clean for qPCR reverse transcription kit. The reverse transcription product was detected with SYBR® Green Premix Pro Taq HS qPCR Kit (Low Rox Plus).

The primers used in qPCR were SEQ ID NOs: 88, 89, 30 and 31.

A reaction system was configured according to the instructions of the SYBR® Green Premix Pro Taq HS qPCR Kit (Rox Plus), and detected by using a QuantStudio™ 5 Real-Time PCR System.

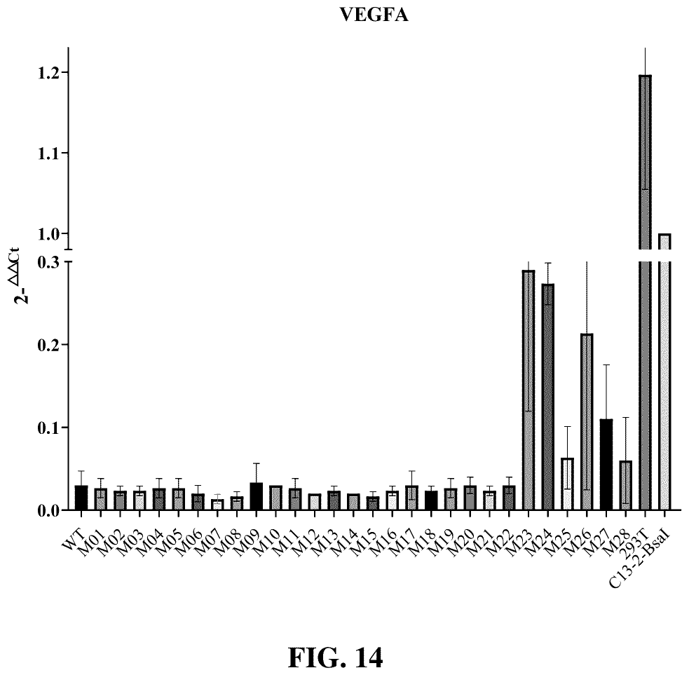

The target RNA level was calculated by the 2-44Ct method. The experiment was repeated in triplicate, and the average results were taken, as shown in Table 26 and FIG. 14.

TABLE 26
VEGFA RNA level after target editing
with the mutant by qPCR test

| | Relative amount of VEGFA |
|---|---|
| Group | RNA after editing |
| C13-2-BsaI | 1.00 |
| WT | 0.03 |
| M01 | 0.03 |
| M02 | 0.02 |
| M03 | 0.02 |
| M04 | 0.02 |
| M05 | 0.02 |
| M06 | 0.02 |
| M07 | 0.02 |
| M08 | 0.02 |
| M09 | 0.03 |
| M10 | 0.03 |
| M11 | 0.03 |
| M12 | 0.02 |
| M13 | 0.02 |
| M14 | 0.02 |
| M15 | 0.02 |
| M16 | 0.02 |
| M17 | 0.03 |
| M18 | 0.02 |
| M19 | 0.03 |
| M20 | 0.03 |
| M21 | 0.02 |
| M22 | 0.03 |
| M23 | 0.29 |
| M24 | 0.27 |
| M25 | 0.07 |
| M26 | 0.21 |
| M27 | 0.11 |
| M28 | 0.06 |
| 293T | 1.20 |

It could be seen from the qPCR test results that all site-mutated mutants retained high editing activity.

### RNAseq Sequencing

The total RNA sample extracted after editing was subject to RNAseq sequencing, with the type of library was LncRNA strand-specific library, the data amount of sequencing was 16 G, and the sequencing strategy was PE150.

### Principle of RNAseq Analysis:

Quality control was performed with fastqc and multiqc, and reads of low-quality were removed y fastp.

Removal was performed by alignment to human rRNA sequences, and the alignment was made by Hisat2 alignment software to hg38 reference genome.

Quantification of genes at expression level was performed by Kallisto software after alignment, and then the variation analysis of expression levels was conducted by sleuth software, wherein a gene with |b| >0.5, qval<0.05 and mean_obs >2 was taken as the differential expression gene; 293T cell control group was taken as the benchmark.

The guide sequence was aligned to reference cDNA by using EMBOSS water software, and the transcript with aligned base no.>=18, mismatched base no.<=6 and the minimum consecutive paired bases no.>=8 was considered as the transcript predicting off-target, and the corresponding gene was considered as potential off-target gene predicted.

The intersection between the differential expression gene significantly down-regulated and potential off-target gene predicted was taken, on-target VEGFA gene was removed, and the off-target gene set was obtained.

### RNAseq Results Analysis

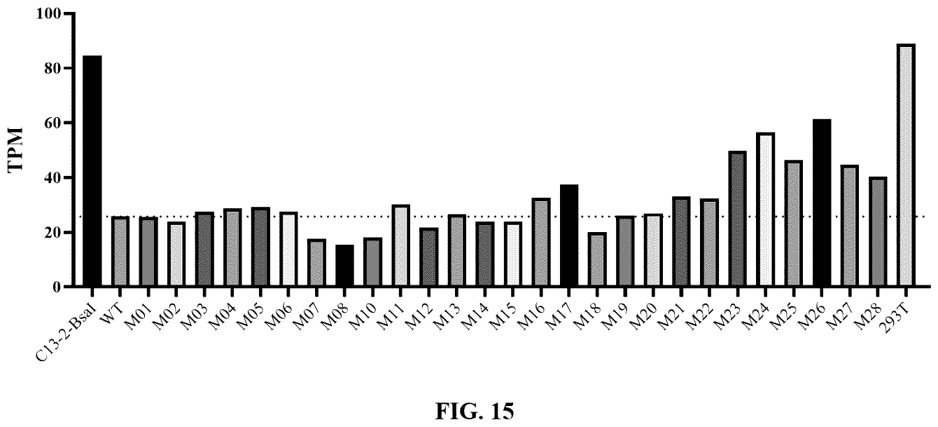

293T cell control group was taken as the benchmark, and the expression level of VEGFA gene of each group was analyzed, and the results were as shown in Table 27 and FIG. 15.

TABLE 27
VEGFA editing efficiency determined by RNAseq

| Groups | TPM value | Editing Efficiency (%) |
|---|---|---|
| C13-2-BsaI | 84.49 | 4.99 |
| 293T | 88.93 | 0 |
| WT | 25.71 | 71.09 |
| M01 | 25.58 | 71.24 |
| M02 | 23.81 | 73.22 |
| M03 | 27.48 | 69.09 |
| M04 | 28.82 | 67.59 |
| M05 | 29.26 | 67.09 |
| M06 | 27.44 | 69.15 |
| M07 | 17.67 | 80.13 |
| M08 | 15.33 | 82.76 |
| M10 | 18.21 | 79.53 |
| M11 | 30.23 | 66.01 |
| M12 | 21.77 | 75.52 |
| M13 | 26.53 | 70.17 |
| M14 | 24.03 | 72.98 |
| M15 | 23.99 | 73.02 |
| M16 | 32.67 | 63.27 |
| M17 | 37.50 | 57.83 |
| M18 | 20.00 | 77.51 |
| M19 | 26.05 | 70.71 |
| M20 | 26.91 | 69.74 |
| M21 | 33.01 | 62.88 |
| M22 | 32.30 | 63.68 |
| M23 | 49.79 | 44.01 |
| M24 | 56.57 | 36.39 |
| M25 | 46.40 | 47.82 |
| M26 | 61.39 | 30.97 |
| M27 | 44.77 | 49.66 |
| M28 | 40.39 | 54.58 |

The result data of RNAseq were substantially consistent with the results of qPCR.

When combined with gRNA in this example, the editing activity of mutants M02, M07, M08, M10, M12, M14, M15 and M18 was slightly increased compared to that of wild-type C13-2.

The number of the differential expression gene and the number of the off-target gene were as shown in Table 28.

In terms of the number of the differential expression gene which was down-regulated in the cell after editing, it is less in M04, M09, M17, M22, M25, M27 and M28 group than that in WT group.

In terms of the number of the off-target gene which was determined after the intersection was taken, it is 0 in M01 group to M28 group, that is, no off-target occurred.

TABLE 28
Differential expression gene and off-target
gene targeting VEGFA of different mutants

| | | | No. of the off-target |
|---|---|---|---|
| | | | gene determined after |
| | Up-regulated | Down-regulated | the intersection was |
| | differential | differential | taken (VEGFA gene had |
| Group | expression gene | expression gene | already been removed) |
| WT | 59 | 3 | 0 |
| M01 | 89 | 8 | 0 |
| M02 | 107 | 12 | 0 |
| M03 | 168 | 27 | 0 |
| M04 | 62 | 1 | 0 |
| M05 | 131 | 3 | 0 |
| M06 | 109 | 15 | 0 |
| M07 | 88 | 5 | 0 |
| M08 | 193 | 31 | 0 |
| M09 | 43 | 2 | 0 |
| M10 | 90 | 6 | 0 |
| M11 | 108 | 7 | 0 |
| M12 | 109 | 4 | 0 |
| M13 | 123 | 4 | 0 |
| M14 | 112 | 4 | 0 |
| M15 | 110 | 20 | 0 |
| M16 | 175 | 16 | 0 |
| M17 | 125 | 2 | 0 |
| M18 | 57 | 3 | 0 |
| M19 | 111 | 10 | 0 |
| M20 | 124 | 5 | 0 |
| M21 | 159 | 5 | 0 |
| M22 | 76 | 2 | 0 |
| M23 | 81 | 9 | 0 |
| M24 | 89 | 8 | 0 |
| M26 | 45 | 4 | 0 |
| M28 | 151 | 1 | 0 |

### Example 14: Second Round of Mutation of C13-2

### Mutants Design

Based on the five mutation sites with low off-target (M09 N87A, M17 R308A, M28 N394A, M04 R47A and M13 R290A) obtained on the basis of the results of Example 1, internal combination or combination with other mutation site among the five sites was designed. In addition, conservative mutations were designed at conservative positions. It was as shown in Table 29.

TABLE 29
Designed mutants in the second round

| Mutant | Mutation |
|---|---|
| M2-1 | R47A + R290A |
| M2-2 | R47A + R314A |
| M2-3 | R290A + R314A |
| M2-4 | R47A + R290A + R314A |
| M2-5 | R308A + N68A |
| M2-6 | N394A + N68A |
| M2-7 | N87A + N68A |
| M2-8 | R308A + N265A |
| M2-9 | N394A + N265A |
| M2-10 | N87A + N265A |
| M2-11 | R308A + N68A + N265A |
| M2-12 | N87A + N68A + N265A |
| M2-13 | T7S |
| M2-14 | A16S |
| M2-15 | S260E |
| M2-16 | A263K |
| M2-17 | M266I |
| M2-18 | N274K |
| M2-19 | F288Y |
| M2-20 | M302F |
| M2-21 | N303S |
| M2-22 | L304I |
| M2-23 | V305K |
| M2-24 | I311M |
| M2-25 | D313E |
| M2-26 | H324Y |
| M2-27 | P326S |
| M2-28 | H327V |
| M2-29 | N332Y |
| M2-30 | N346D |
| M2-31 | T353L |
| M2-32 | T360S |
| M2-33 | E365D |
| M2-34 | A373E |
| M2-35 | M380K |
| M2-36 | S382R |
| M2-37 | K395G |
| M2-38 | Y396D |
| M2-39 | D402L |
| M2-40 | D411E |
| M2-41 | S418K |

### Construction of Verified Vectors

The second round of mutation was tested with target human AR (Androgen Receptor), and C13-2-AR-h3 plasmid vector was synthesized (SEQ ID NO: 161, could express wild-type C13-2 and h3 gRNA targeting AR, and the guide sequence was SEQ ID NO: 162, i.e., ATAACATTTCCGAAGACGACAAGAT).

Site Mutation Kit Mut Express II Fast Mutagenesis Kit V2 from Vazyme was used to modify the C13-2-AR-h3 plasmid vector, and the expression constructs (verified vectors) for each mutant were obtained for expressing the C13-2 mutant and h3 gRNA. Primers used were as shown in Table 30 below.

TABLE 30
Primer used for construction
of expression vectors of mutants

| | | SEQ |
|---|---|---|
| Mutant | Primer Sequence | ID NO |
| M2-1 | TACCACGGCgcAGCCCTGGAGACACCCAAGC | 163 |
| | GGTGAATgcGTAGAACTCGTCGGTGATCTGCT | 164 |
| | GAGTTCTACgcATTCACCATCAGAAAGGACGGC | 165 |
| | CAGGGCTgcGCCGTGGTAGCCCTCGGT | 166 |
| M2-2 | TACCACGGCgcAGCCCTGGAGACACCCAAGC | 167 |
| | CGTATgcGTCGATGATGATCTCTCTGATCTTCA | 168 |
| | TCATCATCGACgcATACGCCAGCGGCCTGAG | 169 |
| | CAGGGCTgcGCCGTGGTAGCCCTCGGT | 170 |
| M2-3 | GAGTTCTACgcATTCACCATCAGAAAGGACGGC | 171 |
| | CGTATgcGTCGATGATGATCTCTCTGATCTTCA | 172 |
| | TCATCATCGACgcATACGCCAGCGGCCTGAG | 173 |
| | GGTGAATgcGTAGAACTCGTCGGTGATCTGCT | 174 |
| M2-4 | TACCACGGCgcAGCCCTGGAGACACCCAAGC | 175 |
| | GGTGAATgcGTAGAACTCGTCGGTGATCTGCT | 176 |
| | GAGTTCTACgcATTCACCATCAGAAAGGACGGC | 177 |
| | CGTATgcGTCGATGATGATCTCTCTGATCTTCA | 178 |
| | TCATCATCGACgcATACGCCAGCGGCCTGAG | 179 |
| | CAGGGCTgcGCCGTGGTAGCCCTCGGT | 180 |
| M2-5 | AAGAATCGACGAGgcCGTGGACCTGTGCGGCGA | 181 |
| | GATCTCTgcGATCTTCACCAGGTTCATGCCC | 182 |
| | GTGAAGATCgcAGAGATCATCATCGACAGATACGC | 183 |
| | ACGgcCTCGTCGATTCTTCTCACCTCG | 184 |
| M2-6 | AAGAATCGACGAGgcCGTGGACCTGTGCGGCGA | 185 |
| | AGGATGTACTTGgcCTTGGGGTCGTTGAAGTAGGG | 186 |
| | CAAGgcCAAGTACATCCTGAAGTACAAGGACG | 187 |
| | ACGgcCTCGTCGATTCTTCTCACCTCG | 188 |
| M2-7 | AAGAATCGACGAGgcCGTGGACCTGTGCGGCGA | 189 |
| | TCTCGCTGGGGgcCACCAGCAGGGCCTCGAT | 190 |
| | TGGTGgcCCCCAGCGAGAAGGTGGG | 191 |
| | ACGgcCTCGTCGATTCTTCTCACCTCG | 192 |
| M2-8 | AAGAACGCCGTGgcCATGGCCATCCTGTTCGACC | 193 |
| | GATCTCTgcGATCTTCACCAGGTTCATGCCC | 194 |
| | GTGAAGATCgcAGAGATCATCATCGACAGATACGC | 195 |
| | CATGgcCACGGCGTTCTTGCTCAGG | 196 |
| M2-9 | AAGAACGCCGTGgcCATGGCCATCCTGTTCGACC | 197 |
| | AGGATGTACTTGgcCTTGGGGTCGTTGAAGTAGGG | 198 |
| | CAAGgcCAAGTACATCCTGAAGTACAAGGACG | 199 |
| | CATGgcCACGGCGTTCTTGCTCAGG | 200 |
| M2-10 | TGGTGgcCCCCAGCGAGAAGGTGGG | 201 |
| | CATGgcCACGGCGTTCTTGCTCAGG | 202 |
| | AAGAACGCCGTGgcCATGGCCATCCTGTTCGACC | 203 |
| | TCTCGCTGGGGgcCACCAGCAGGGCCTCGAT | 204 |
| M2-11 | AAGAATCGACGAGgcCGTGGACCTGTGCGGCGA | 205 |
| | CATGgcCACGGCGTTCTTGCTCAGG | 206 |
| | AAGAACGCCGTGgcCATGGCCATCCTGTTCGACC | 207 |
| | GATCTCTgcGATCTTCACCAGGTTCATGCCC | 208 |
| | GTGAAGATCgcAGAGATCATCATCGACAGATACGC | 209 |
| | ACGgcCTCGTCGATTCTTCTCACCTCG | 210 |
| M2-12 | AAGAATCGACGAGgcCGTGGACCTGTGCGGCGA | 211 |
| | TCTCGCTGGGGgcCACCAGCAGGGCCTCGAT | 212 |
| | TGGTGgcCCCCAGCGAGAAGGTGGG | 213 |
| | CATGgcCACGGCGTTCTTGCTCAGG | 214 |
| | AAGAACGCCGTGgcCATGGCCATCCTGTTCGACC | 215 |
| | ACGgcCTCGTCGATTCTTCTCACCTCG | 216 |
| M2-13 | ACAAGAAAtCCAAGGCCAAGAGAATGGGCGTG | 217 |
| | GGCCTTGGaTTTCTTGTCCTTGCTCATGTCGA | 218 |
| M2-14 | AGAATGGGCGTGAAGtCCCTGCTGGCCCACGGCG | 219 |
| | GaCTTCACGCCCATTCTCTTGGCCTTGGTTTT | 220 |
| M2-15 | CTTCCTGgaaAAGAACGCCGTGAACATGGCCA | 221 |
| | CGTTCTTttcCAGGAAGTCCTTGTTCACCTTCTT | 222 |
| M2-16 | GCAAGAACaagGTGAACATGGCCATCCTGTTCG | 223 |
| | GTTCACcttGTTCTTGCTCAGGAAGTCCTTGTT | 224 |
| M2-17 | GTGAACATaGCCATCCTGTTCGACCTGCTGAA | 225 |
| | AGGATGGCtATGTTCACGGCGTTCTTGCTCAG | 226 |
| M2-18 | TGTTCGACCTGCTGAAgGCCAGAGACGTGGAGCAGAA | 227 |
| | cTTCAGCAGGTCGAACAGGATGGCCATGTTCA | 228 |
| M2-19 | CGACGAGTaCTACAGATTCACCATCAGAAAGGACG | 229 |
| | ATCTGTAGtACTCGTCGGTGATCTGCTTCTTC | 230 |
| M2-20 | AACCTGGGCttcAACCTGGTGAAGATCAGAGAGATCA | 231 |
| | AGGTTgaaGCCCAGGTTCTTGCCGTCCTTTCT | 232 |
| M2-21 | CATGAgCCTGGTGAAGATCAGAGAGATCATCA | 233 |
| | TCTTCACCAGGcTCATGCCCAGGTTCTTGCCG | 234 |
| M2-22 | GCATGAACatcGTGAAGATCAGAGAGATCATCATCGA | 235 |
| | CTTCACgatGTTCATGCCCAGGTTCTTGCCGT | 236 |
| M2-23 | GGCATGAACCTGaaGAAGATCAGAGAGATCATCATCGA | 237 |
| | CA | |
| | CTTCttCAGGTTCATGCCCAGGTTCTTGCCGTC | 238 |
| M2-24 | GAGATCATgATCGACAGATACGCCAGCGGCCT | 239 |
| | CTGTCGATCATGATCTCTCTGATCTTCACCAGGT | 240 |
| M2-25 | ATCATCGAgAGATACGCCAGCGGCCTGAGAGA | 241 |
| | GCGTATCTcTCGATGATGATCTCTCTGATCTTCA | 242 |
| M2-26 | ACAAGAAGtACGACCCCCACAGACAGAAGATC | 243 |
| | GGGGTCGTaCTTCTTGTCTCTCAGGCCGCTGG | 244 |
| M2-27 | AAGAAGCACGACtCCCACAGACAGAAGATCAACGTG | 245 |
| | TGGGaGTCGTGCTTCTTGTCTCTCAGGCCGCT | 246 |
| M2-28 | CCgtCAGACAGAAGATCAACGTGATCGCCGACT | 247 |
| | TGATCTTCTGTCTGacGGGGTCGTGCTTCTTGTCTCT | 248 |
| M2-29 | GAAGATCtACGTGATCGCCGACTTCCTGATCT | 249 |
| | CGATCACGTaGATCTTCTGTCTGTGGGGGTCG | 250 |
| M2-30 | CAGgACCAGGGCATCATCGACAAGACCGTGAG | 251 |
| | ATGATGCCCTGGTcCTGGCTCAGGGCTCTGAAGA | 252 |
| M2-31 | TCATCGACAAGctCGTGAGCAGCCTGAGACTGAC | 253 |
| | TCACGagCTTGTCGATGATGCCCTGGTTCTGGC | 254 |
| M2-32 | TGAGACTGtCCAAGGACGAGGAGGAGAAGGAC | 255 |
| | GTCCTTGGaCAGTCTCAGGCTGCTCACGGTCT | 256 |
| M2-33 | AAGGACGAGGAGGAcAAGGACCACGTGTACCAGAACG | 257 |
| | TTgTCCTCCTCGTCCTTGGTCAGTCTCAGGCT | 258 |
| M2-34 | ACGTGTACCAGAACGagGCCGAGCTGGTGTGGGGC | 259 |
| | CctCGTTCTGGTACACGTGGTCCTTCTCCTCCT | 260 |
| M2-35 | AaGGTGAGCAACTGCCTGACCCCCTACTTCAA | 261 |
| | AGGCAGTTGCTCACCtTGCCCCACACCAGCTCGG | 262 |
| M2-36 | ATGGTGAGgAACTGCCTGACCCCCTACTTCAA | 263 |
| | AGGCAGTTcCTCACCATGCCCCACACCAGCTC | 264 |
| M2-37 | CAAGAACggGTACATCCTGAAGTACAAGGACGC | 265 |
| | AGGATGTACccGTTCTTGGGGTCGTTGAAGTAGG | 266 |
| M2-38 | AGAACAAGgACATCCTGAAGTACAAGGACGCC | 267 |
| | CAGGATGTcCTTGTTCTTGGGGTCGTTGAAGT | 268 |
| M2-39 | AGTACAAGctCGCCAAGACCCCCGGCGACTTCG | 269 |
| | TCTTGGCGagCTTGTACTTCAGGATGTACTTGTTCTTG | 270 |
| M2-40 | TTCGAGGAgTGGATCACCAGCAAGATCAGCGA | 271 |
| | GTGATCCAcTCCTCGAAGTCGCCGGGGGTCTT | 272 |
| M2-41 | CAAGATCAagGAGGACGACGGCGAGCCCTTCGT | 273 |
| | TCGTCCTCctTGATCTTGCTGGTGATCCAGTCC | 274 |

### Transfection

The 293T cells were transfected with verified vectors and control vectors according to the instructions of Lipofectamine™ 2000 (Thermo). The C13-2-BsaI control group was transfected with the C13-2-BsaI vector in previous examples, the WT control group was transfected with the C13-2-AR-h3 vector, both of which expressed wild-type C13-2. A 293T cell control group was set additionally, without transfection with any plasmid. Detection of RNA level with qPCR

At 48 h after transfection, the cells were subjected to RNA extraction with a SteadyPure Universal RNA Extraction Kit, and the RNA concentration was detected with an ultramicro spectrophotometer. The RNA product was subject to reverse transcription by using the Evo M-MLV Mix Kit with gDNA Clean for qPCR reverse transcription kit. The reverse transcription product was detected with SYBR® Green Premix Pro Taq HS qPCR Kit (Low Rox Plus).

Primers used in qPCR were as follows:

Detection of AR:
(SEQ ID NO: 275)
CCAGGGACCATGTTTTGCC
(SEQ ID NO: 276)
CGAAGACGACAAGATGGACAA

Detection of internal reference GAPDH: SEQ ID NOs: 30 and 31.

A reaction system was configured according to the instructions of the SYBR® Green Premix Pro Taq HS qPCR Kit (Rox Plus), and detected by using a QuantStudio™ 5 Real-Time PCR System.

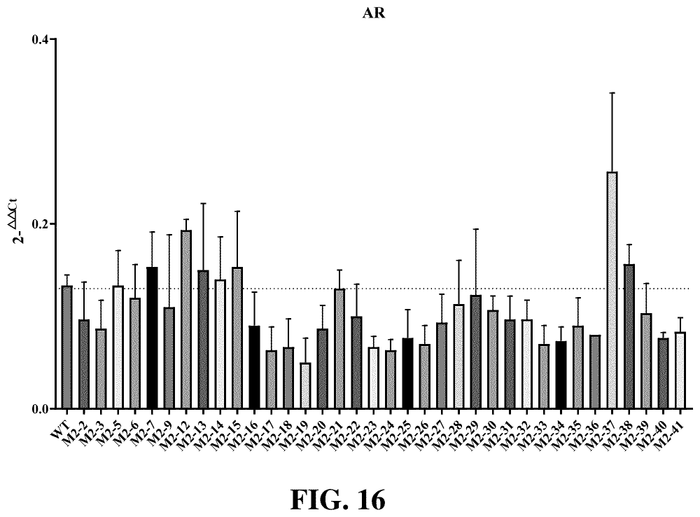

The target RNA level was calculated by the 2-AACt method. The experiment was repeated in triplicate, and the average results were taken, as shown in Table 31 and FIG. 16.

TABLE 31
AR RNA level after target editing with the mutant by qPCR test

| | Relative amount of AR |
|---|---|
| Group | RNA after editing |
| BsaI | 1.00 |
| WT2 | 0.13 |
| M2-2 | 0.09 |
| M2-3 | 0.09 |
| M2-5 | 0.14 |
| M2-6 | 0.12 |
| M2-7 | 0.15 |
| M2-9 | 0.11 |
| M2-12 | 0.19 |
| M2-13 | 0.15 |
| M2-14 | 0.14 |
| M2-15 | 0.15 |
| M2-16 | 0.09 |
| M2-17 | 0.07 |
| M2-18 | 0.07 |
| M2-19 | 0.05 |
| M2-20 | 0.09 |
| M2-21 | 0.13 |
| M2-22 | 0.10 |
| M2-23 | 0.07 |
| M2-24 | 0.06 |
| M2-25 | 0.08 |
| M2-26 | 0.07 |
| M2-27 | 0.10 |
| M2-28 | 0.11 |
| M2-29 | 0.12 |
| M2-30 | 0.10 |
| M2-31 | 0.10 |
| M2-32 | 0.10 |
| M2-33 | 0.07 |
| M2-34 | 0.07 |
| M2-35 | 0.09 |
| M2-36 | 0.08 |
| M2-37 | 0.26 |
| M2-38 | 0.16 |
| M2-39 | 0.10 |
| M2-40 | 0.07 |
| M2-41 | 0.08 |
| 293T | 1.21 |

### RNAseq Sequencing

The total RNA sample extracted after editing was subject to RNAseq sequencing, with the type of library to be constructed was LncRNA strand-specific library, the data amount of sequencing was 16 G, and the sequencing strategy was PE150.

### Principle of RNAseq Analysis:

Quality control was performed with fastqc and multiqc, and reads of low-quality were removed y fastp.

Removal was performed by alignment to human rRNA sequences, and the alignment was made by Hisat2 alignment software to hg38 reference genome.

Quantification of genes at expression level was performed by Kallisto software after alignment, and then the variation analysis of expression levels was conducted by sleuth software, wherein a gene with |b| >0.5, qval<0.05 and mean_obs >2 was taken as the differential expression gene.

The guide sequence was aligned to reference cDNA by using EMBOSS water software, and the transcript with aligned base no.>=18, mismatched base no.<=6 and the minimum consecutive paired bases no.>=8 was considered as the transcript predicting off-target, and the corresponding gene was considered as potential off-target gene predicted.

The intersection between the differential expression gene significantly down-regulated and potential off-target gene predicted was taken, on-target VEGFA gene was removed, and the off-target gene set was obtained.

RNAseq Results Analysis

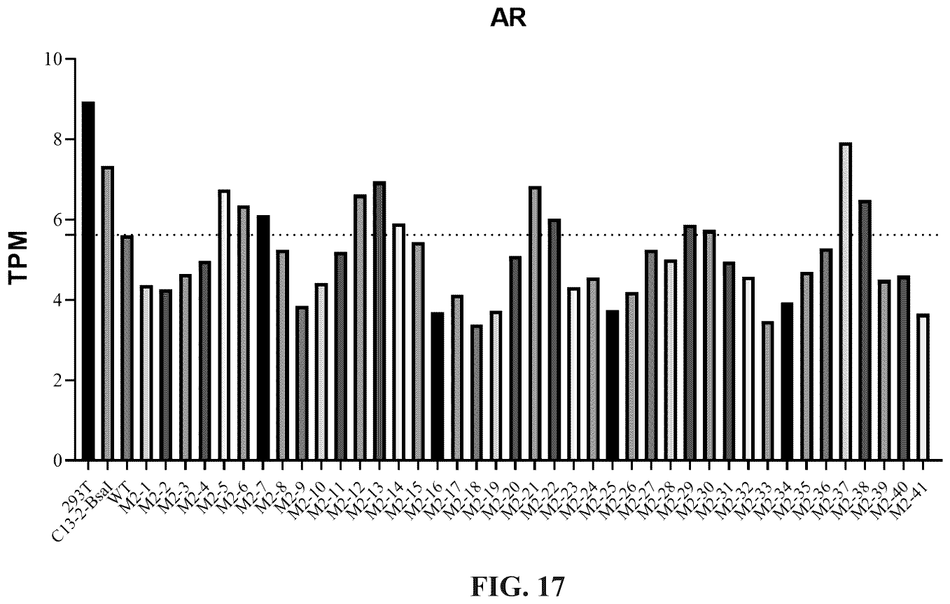

293T cell control group was taken as the benchmark, and the expression level and editing efficiency (average was taken) of AR gene of each group was analyzed, and the results were as shown in Table 32 and FIG. 17.

TABLE 32
AR editing efficiency determined by RNAseq

| Groups | TPM value | Editing Efficiency (%) |
|---|---|---|
| C13-2-BsaI | 7.34 | 17.91 |
| 293T | 8.94 | 0 |
| WT | 5.62 | 37.17 |
| M2-1 | 4.36 | 51.28 |
| M2-2 | 4.27 | 52.2 |
| M2-3 | 4.65 | 47.99 |
| M2-4 | 4.98 | 44.28 |
| M2-5 | 6.75 | 24.54 |
| M2-6 | 6.36 | 28.91 |
| M2-7 | 6.12 | 31.61 |
| M2-8 | 5.24 | 41.38 |
| M2-9 | 3.85 | 56.96 |
| M2-10 | 4.42 | 50.56 |
| M2-11 | 5.20 | 41.82 |
| M2-12 | 6.62 | 25.96 |
| M2-13 | 6.95 | 22.34 |
| M2-14 | 5.90 | 34.01 |
| M2-15 | 5.44 | 39.18 |
| M2-16 | 3.69 | 58.71 |
| M2-17 | 4.13 | 53.8 |
| M2-18 | 3.38 | 62.18 |
| M2-19 | 3.73 | 58.25 |
| M2-20 | 5.10 | 42.96 |
| M2-21 | 6.83 | 23.58 |
| M2-22 | 6.02 | 32.7 |
| M2-23 | 4.32 | 51.74 |
| M2-24 | 4.56 | 49.02 |
| M2-25 | 3.74 | 58.2 |
| M2-26 | 4.19 | 53.18 |
| M2-27 | 5.24 | 41.43 |
| M2-28 | 5.00 | 44.06 |
| M2-29 | 5.87 | 34.42 |
| M2-30 | 5.74 | 35.81 |
| M2-31 | 4.95 | 44.66 |
| M2-32 | 4.58 | 48.76 |
| M2-33 | 3.47 | 61.22 |
| M2-34 | 3.93 | 56.1 |
| M2-35 | 4.69 | 47.52 |
| M2-36 | 5.29 | 40.84 |
| M2-37 | 7.93 | 11.32 |
| M2-38 | 6.49 | 27.47 |
| M2-39 | 4.50 | 49.73 |
| M2-40 | 4.60 | 48.62 |
| M2-41 | 3.65 | 59.17 |

The result data of RNAseq were substantially consistent with the results of qPCR.

When combined with gRNA in this example, the editing activity of mutants M2-1, M2-2, M2-3, M2-4, M2-9, M2-10, M2-16, M2-17, M2-18, M2-19, M2-23, M2-24, M2-25, M2-26, M2-32, M2-33, M2-34, M2-35, M2-39, M2-40 and M2-41 was higher than that of wild-type C13-2.

The number of the differential expression gene and the number of the off-target gene were as shown in Table 33.

In terms of the number of the differential expression gene which was down-regulated in the cell after editing, it is less in M2-1, M2-2, M2-3, M2-4, M2-5, M2-6, M2-7, M2-8, M2-10, M2-13, M2-14, M2-15, M2-16, M2-21, M2-22, M2-23, M2-24, M2-25, M2-26, M2-27, M2-28, M2-29, M2-30, M2-31, M2-32, M2-33, M2-34, M2-35, M2-36, M2-37, M2-38, M2-39, M2-40 and M2-41 groups than that in WT group.

In terms of the number of the off-target gene which was determined after the intersection was taken, it is less in M2-1, M2-2, M2-3, M2-4, M2-5, M2-6, M2-7, M2-8, M2-10, M2-13, M2-14, M2-15, M2-16, M2-21, M2-22, M2-23, M2-24, M2-25, M2-26, M2-27, M2-28, M2-29, M2-30, M2-31, M2-32, M2-33, M2-35, M2-36, M2-37, M2-38, M2-39, M2-40 and M2-41 groups than that in WT group. Among them, no off-target occurred in M2-1, M2-6, M2-7, M2-14, M2-15, M2-22, M2-31, M2-38 and M2-39 groups.

TABLE 33
Differential expression gene and off-target gene
targeting AR of mutants

| | | | No. of the off-target |
|---|---|---|---|
| | | | gene determined after |
| | Up-regulated | Down-regulated | the intersection was |
| | differential | differential | taken (AR gene had |
| Group | expression gene | expression gene | already been removed) |
| C13-2- | 4 | 42 | N/A |
| BsaI | | | |
| WT | 1199 | 1023 | 54 |
| M2-1 | 42 | 4 | 0 |
| M2-2 | 987 | 670 | 40 |
| M2-3 | 55 | 5 | 1 |
| M2-4 | 497 | 114 | 4 |
| M2-5 | 1080 | 923 | 39 |
| M2-6 | 254 | 18 | 0 |
| M2-7 | 55 | 2 | 0 |
| M2-8 | 914 | 633 | 24 |
| M2-9 | 1439 | 1508 | 68 |
| M2-10 | 1195 | 942 | 42 |
| M2-11 | 2344 | 2156 | 102 |
| M2-12 | 1426 | 1366 | 75 |
| M2-13 | 775 | 547 | 24 |
| M2-14 | 41 | 5 | 0 |
| M2-15 | 85 | 3 | 0 |
| M2-16 | 535 | 165 | 9 |
| M2-17 | 1225 | 1055 | 57 |
| M2-18 | 1551 | 1255 | 88 |
| M2-19 | 1481 | 1404 | 81 |
| M2-20 | 1332 | 1264 | 58 |
| M2-21 | 301 | 38 | 2 |
| M2-22 | 39 | 3 | 0 |
| M2-23 | 193 | 14 | 1 |
| M2-24 | 842 | 318 | 21 |
| M2-25 | 908 | 624 | 31 |
| M2-26 | 1221 | 820 | 50 |
| M2-27 | 974 | 457 | 24 |
| M2-28 | 755 | 443 | 20 |
| M2-29 | 473 | 122 | 8 |
| M2-30 | 886 | 490 | 20 |
| M2-31 | 110 | 3 | 0 |
| M2-32 | 694 | 295 | 14 |
| M2-33 | 766 | 417 | 18 |
| M2-34 | 1149 | 959 | 57 |
| M2-35 | 1129 | 861 | 44 |
| M2-36 | 592 | 397 | 20 |
| M2-37 | 306 | 50 | 2 |
| M2-38 | 51 | 4 | 0 |
| M2-39 | 101 | 2 | 0 |
| M2-40 | 828 | 348 | 16 |
| M2-41 | 837 | 385 | 21 |

### Example 15: Test at Target Protein Level

The 293T cells were transfected (Lipofectamine™ 2000, Thermo Fisher) with the C13-2-VEGFA vector and the negative control vector C13-2-BsaI in previous examples, cultured at 37° C. for 72h, and the supernatant was collected. The VEGFA protein level was detected with Human VEGF-A (Vascular Endothelial Cell Growth Factor A) ELISA Kit from Elascience, and the results showed that the VEGFA protein expression reduced by 97.4% compared to that of the negative control group.

In 293T cells, after editing by CasRx expressed by the vector and the gRNA comprising the same guide sequence, the VEGFA protein expression reduced by 75.7% compared to that of CasRx negative control group.

What is claimed is:

1. A non-naturally occurring or engineered guide polynucleotide comprising (i) a direct repeat sequence having at least 70% sequence identity compared to any one of SEQ ID NO: 3 and SEQ ID NOs: 80-87, wherein the direct repeat sequence is linked to (ii) a guide sequence engineered to hybridize with a target RNA, wherein the guide polynucleotide can form a CRISPR complex with a Cas13 protein and guide the sequence-specific binding of the CRISPR complex to the target RNA.

2. The guide polynucleotide according to claim 1, wherein the base at position 26 corresponding to SEQ ID NO: 3 in the direct repeat sequence is A.

3. The guide polynucleotide according to claim 1, wherein the direct repeat sequence is
(SEQ ID NO: 277)
GGAAGATN₁ACTCTACAAACCTGTAGN₂GN₃N₄N₅N₆N₇N₈N₉N₁₀N₁₁;
wherein N₁ and N₃-N₁₁ are independently any one selected from A, C, G, and T; and N₂ is any one selected from A and G;
or, the direct repeat sequence is GGAAGATN12ACTCTACAAACCTGTAGN13GN14N₁₅N₁₆N₁₇N₁₈N₁₉N₂₀N₂₁N₂₂ (SEQ ID NO: 278; wherein N₁₂, N₁₃, N₁₉, and N₂₁ are independently any one selected from A and G, N₁₄ is any one selected from A and T, N₁₅ and N₁₆ are independently any one selected from C and T, N₁₇ and N₁₈ are independently any one selected from G and T, and N₂₀ and N₂₂ are independently any one selected from C and G.

4. The guide polynucleotide according to claim 1, wherein the direct repeat sequence has at least 80%, at least 90%, or at least 95% sequence identity compared to any one of SEQ ID NOs: 3, 81, 82, 84, and 87;
and/or, the guide sequence is located at the 3′ end of the direct repeat sequence;
and/or, the guide sequence comprises 15-35 nucleotides;
and/or, the guide sequence hybridizes with the target RNA with no more than one nucleotide mismatch;
and/or, the direct repeat sequence comprises 25 to 40 nucleotides;
and/or, the guide polynucleotide further comprises an aptamer sequence;
and/or, the guide polynucleotide comprises a nucleotide with modification;
and/or, the amino acid sequence of the Cas13 protein has at least 90% sequence identity compared to SEQ ID NO: 1.

5. The guide polynucleotide according to claim 4, wherein the aptamer sequence is inserted into a loop of the guide polynucleotide;
and/or, the aptamer sequence comprises an MS2 aptamer sequence, a PP7 aptamer sequence, or a Qβ aptamer sequence;
and/or, wherein the modification comprises 2′-O-methyl, 2′-O-methyl-3′-phosphorothioate, or 2′-O-methyl-3′-thioPACE.

6. The guide polynucleotide according to claim 1, wherein the target RNA is located in nucleus of a eukaryotic cell;
and/or, the target RNA is any one or more selected from TTR RNA, SOD1 RNA, PCSK9 RNA, VEGFA RNA, VEGFR1 RNA, PTBP1 RNA, AQp1 RNA, and ANGPTL3 RNA.

7. A CRISPR-Cas13 system comprising:
(1) a Cas13 protein having the amino acid sequence which has at least 90% sequence identity compared to SEQ ID NO: 1; or a nucleic acid encoding the Cas13 protein; and
(2) the guide polynucleotide according to claim 1 or a nucleic acid encoding the guide polynucleotide;
wherein the guide sequence is engineered to hybridize with a target RNA;
wherein the guide polynucleotide can form a CRISPR complex with the Cas13 protein and guide a sequence-specific binding of the CRISPR complex to the target RNA; wherein the Cas13 protein and the guide polynucleotide do not naturally occur together.

8. The CRISPR-Cas13 system according to claim 7, wherein the target RNA is located in a eukaryotic cell.

9. A vector system comprising the CRISPR-Cas13 system according to claim 7, wherein the vector system comprises one or more vectors comprising a polynucleotide sequence encoding the Cas13 protein and a polynucleotide sequence encoding the guide polynucleotide.

10. The vector system according to claim 9, wherein
the vector is an adeno-associated viral vector comprising a DNA encoding the Cas13 protein and the guide polynucleotide;
or, the vector is a lipid nanoparticle comprising the guide polynucleotide and an mRNA encoding the Cas13 protein;
and/or, the vector is a lentiviral vector comprising the guide polynucleotide and an mRNA encoding the Cas13 protein.

11. A ribonucleoprotein complex comprising the CRISPR-Cas13 system according to claim 7, wherein the ribonucleoprotein complex is formed from the guide polynucleotide and the Cas13 protein.

12. A viral-like particle comprising the CRISPR-Cas13 system according to claim 7, wherein the viral-like particle comprises a ribonucleoprotein complex formed from the guide polynucleotide and the Cas13 protein; optionally, the Cas13 protein is fused to a gag protein.

13. A pharmaceutical composition comprising the CRISPR-Cas13 system according to claim 7.

14. An in vitro composition comprising the CRISPR-Cas13 system according to claim 7 and a labeled detector RNA that is not capable of hybridization with the guide polynucleotide.

15. A method for detecting the target RNA in a nucleic acid sample suspected to comprising the target RNA, wherein the method comprises the step of contacting the CRISPR-Cas13 system according to claim 7 with the nucleic acid sample.

16. A method for realizing any one of the following:
cleaving one or more target RNA molecules or nicking one or more target RNA molecules; activating or upregulating one or more target RNA molecules; activating or inhibiting translation of one or more target RNA molecules; inactivating one or more target RNA molecules; visualizing, labelling or detecting one or more target RNA molecules; binding one or more target RNA molecules; transporting one or more target RNA molecules; and masking one or more target RNA molecules;
wherein the method comprises the step of contacting the CRISPR-Cas13 system according to claim 7 with the target RNA.

17. A method for diagnosing, treating, or preventing diseases or disorders associated with a target RNA, comprising administering the CRISPR-Cas13 system according to claim 7 to a sample of a subject in need thereof, or to a subject in need thereof;
wherein the target RNA is selected from TTR RNA, SOD1 RNA, PCSK9 RNA, VEGFA RNA, VEGFR1 RNA, PTBP1 RNA, AQp1 RNA, and ANGPTL3 RNA.


SEQUENCE LISTING (284 sequences)

SEQ ID NO: 1 - protein, 893 aa, synthetic construct
MSKDKKTKAKRMGVKALLAHGEDKLTMTTFGKGNRSKIEFTEGYHGRALETPKHFGIRGFEVRRIDENVDLCGDLEEGKTIEALLVNPSEKVGEDYLKLKGTLEKRFFGREFPHDNIRIQLIYNILDIYKILGMNVADILYALGNMQDTELDIDMFGQSLNNEDNLKECLKRMRPYMGYFGDIFKISPKGENIADREHNKKVLRCISVLRNATAHDKQDEYPWFKSSDIYETKIFKADMWKIIKDQYREKIKKVNKDFLSKNAVNMAILFDLLNARDVEQKKQITDEFYRFTIRKDGKNLGMNLVKIREIIIDRYASGLRDKKHDPHRQKINVIADFLIFRALSQNQGIIDKTVSSLRLTKDEEEKDHVYQNAAELVWGMVSNCLTPYFNDPKNKYILKYKDAKTPGDFEDWITSKISEDDGEPFVKVLSFLCNFLEGKEINELLTAYIHKFECIQDFLNVISSLGENVQFQPRFALFNNASFAQNVAVQLRILASIGKMKPDLTEAKRPLYKAAIRMLCPPEKWEKYTSDEWLEKNMLLNSEDRKNDKKKKQVNPFRNFIAGNVIESRRFMYLVRYSKPKAVRAIMQNRSIVNYVLHRLPSEQVHRYASVFPENFADLEQEIDFLTKKLFEFSFEELLHEKDVILNNSRSHKPSLEIERLKAITGLYLSVAYIAIKNIVKANARYYIAFAVFERDKELVKAKDARIQTKIPETDFPDYFCLTQYYLDRDEEKKFPGDPRDKEAFFEHLRKTKRHFSKQWREWLNEKIADAKSSQATGLLLREARNDVEHLNVLRAIPDYIQDFRHGEKGETAMNSYFELYHYLMQRLMLKNTELDLSHWSGWIMRSGRPDRDLIQIAFVSLAYNLPRYRNLTKEHHFDDTVLQKIREKESLD

SEQ ID NO: 2 - protein, 966 aa, synthetic construct
IEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 3 - RNA, 36 nt, synthetic construct
  source 1..36: mol_type = other RNA
ggaagataactctacaaacctgtagggttctgagac

SEQ ID NO: 4 - RNA, 30 nt, synthetic construct
  source 1..30: mol_type = other RNA
tgccgttcttctgcttgtcggccatgatat

SEQ ID NO: 5 - RNA, 26 nt, synthetic construct
  source 1..26: mol_type = other RNA
agggcagaaccgatgctgatgaagac

SEQ ID NO: 6 - RNA, 30 nt, synthetic construct
  source 1..30: mol_type = other RNA
gtggttggagaactggatgtagatgggctg

SEQ ID NO: 7 - RNA, 48 nt, synthetic construct
  source 1..48: mol_type = other RNA
ccacgaccctctttgtcttcactcgagtgaagacaaagagggtcgtgg

SEQ ID NO: 8 - RNA, 48 nt, synthetic construct
  source 1..48: mol_type = other RNA
cagcccatctacatccagttcctcgaggaactggatgtagatgggctg

SEQ ID NO: 9 - DNA, 2682 bp, synthetic construct
  source 1..2682: mol_type = other DNA
atgagcaaagacaaaaaaacaaaagcgaaacgaatgggagtcaaagccctgttggctcacggggaagataaactgacgatgaccacctttgggaaaggaaaccgttcgaagatcgagtttacggaagggtatcacggccgagcgcttgagacccccaaacactttggaatacgcggctttgaggtaaggaggatcgatgaaaacgtcgatctctgcggggatctcgaggaggggaaaacgatcgaggccttgctggtcaacccgtctgaaaaagtcggcgaggactacctcaagcttaaggggacactggaaaaacgtttcttcggccgtgaatttccgcatgacaacatccggatccagctcatctataatattctcgacatttataagattttggggatgaatgtcgctgatattctttacgcgctgggaaacatgcaggatacagagctggacattgatatgtttggacagtctttgaacaatgaagacaaccttaaagaatgtctgaaacgaatgcggccttatatgggctatttcggagatattttcaagatttccccaaaaggcgagaacatagccgatcgggaacataacaaaaaagttctgcgctgtatttctgttctgcgcaacgctaccgcccatgacaaacaagacgaatatccgtggttcaagagcagcgacatctacgaaacgaaaatttttaaggcggacatgtggaaaatcatcaaggaccaatatcgggaaaagatcaagaaagtcaataaggattttttatcgaaaaacgcggttaacatggccatcctatttgaccttctgaatgcccgtgatgtggaacagaaaaagcagatcacggatgaattctatcgtttcacgattcgaaaagacgggaagaatctggggatgaatctggttaagatccgcgagataattattgatcgttacgccagtggcctccgtgataaaaagcacgatcctcaccgccagaaaatcaatgtgatcgcggacttcctgattttccgcgctctttcgcaaaatcagggaatcatcgacaagaccgtttccagcctgcgcctcacgaaagacgaagaggaaaaagaccacgtgtaccagaatgccgcggaactggtctgggggatggtcagtaattgtttgaccccgtattttaacgaccctaaaaataaatacattcttaaatacaaagacgccaaaactcctggtgacttcgaggattggatcaccagtaaaatctcggaggatgacggggagccgttcgtgaaagtgctttcgttcctctgtaatttcctggaagggaaggaaatcaacgagctgctgaccgcttacattcataaattcgagtgtattcaggacttcttgaacgtgatttccagtcttggggaaaacgttcagtttcagcctcgtttcgcgctgttcaacaatgccagtttcgcccaaaatgttgcggtacagttgcgtatcctggcaagtatcgggaagatgaaacccgatttgaccgaggcgaaacgcccgctttacaaggcagcgattcggatgctttgtcctccagagaagtgggaaaaatacacctcggatgagtggcttgaaaaaaatatgctcctcaattccgaggaccgtaaaaatgataaaaagaagaaacaggtcaaccctttccggaatttcatcgcggggaatgtgatcgagtcacgtcggtttatgtatctggtccggtattcaaagccgaaagcggttcgtgcgattatgcaaaaccggagtatcgtgaactatgttcttcacaggcttccgtccgagcaggttcaccggtacgccagcgtgtttcctgaaaatttcgccgatctggaacaggaaattgacttcttgacaaagaaacttttcgaattctcattcgaggaactcctccatgaaaaggatgtgattttgaataattccagatctcacaaaccttccttagagatcgaacgccttaaagccattacagggctgtacctttcggttgcctacatcgccatcaaaaacatcgtgaaagccaacgcacggtattacatcgccttcgcggttttcgaacgtgacaaggaactggtgaaagccaaggacgcacgaatccagacgaagatccccgagacagattttccggactatttctgcctcacgcagtattaccttgaccgcgatgaggagaaaaaattcccgggtgacccgcgtgataaagaggcttttttcgaacatctccgcaagacgaaacggcatttctcaaaacagtggcgcgagtggctgaatgagaaaattgcggacgcaaagagctcccaggcaaccggccttttgttgagagaggcccgaaacgacgtggaacatttgaacgtcctgcgcgcgatcccggattacatccaggatttccgccatggggaaaaaggggaaacggcaatgaattcgtatttcgagctgtaccattacctcatgcagaggctgatgctcaaaaacacggaattggatttgtcccattggagcggctggatcatgcgttccggtcggccggatcgggatttgattcagatcgcgttcgtttcattggcttacaatctgccgcgctaccgtaatttgacgaaggaacaccatttcgatgatacggtattgcagaaaattcgtgagaaagagtccctagactaa

SEQ ID NO: 10 - DNA, 8182 bp, synthetic construct
  source 1..8182: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgcggatccccggcagctaagaaaaagaaactggatggcagcgtcgacatgagcaaggacaagaaaaccaaggccaagagaatgggcgtgaaggccctgctggcccacggcgaggacaagctgaccatgaccaccttcggcaagggcaacagaagcaagatcgagttcaccgagggctaccacggcagagccctggagacacccaagcacttcggcatcagaggcttcgaggtgagaagaatcgacgagaacgtggacctgtgcggcgacctggaggagggcaagaccatcgaggccctgctggtgaaccccagcgagaaggtgggcgaggactacctgaagctgaagggcaccctggagaagagattcttcggcagagagttcccccacgacaacatcagaatccagctgatctacaacatcctggacatctacaagatcctgggcatgaacgtggccgacatcctgtacgccctgggcaacatgcaggacaccgagctggacatcgacatgttcggccagagcctgaacaacgaggacaacctgaaggagtgcctgaagagaatgaggccctacatgggctacttcggcgacatcttcaagatcagccccaagggcgagaacatcgccgacagagagcacaacaagaaggtgctgagatgcatcagcgtgctgagaaacgccaccgcccacgacaagcaggacgagtacccctggttcaagagcagcgacatctacgagacaaagatcttcaaggccgacatgtggaagatcatcaaggaccagtacagagagaagatcaagaaggtgaacaaggacttcctgagcaagaacgccgtgaacatggccatcctgttcgacctgctgaacgccagagacgtggagcagaagaagcagatcaccgacgagttctacagattcaccatcagaaaggacggcaagaacctgggcatgaacctggtgaagatcagagagatcatcatcgacagatacgccagcggcctgagagacaagaagcacgacccccacagacagaagatcaacgtgatcgccgacttcctgatcttcagagccctgagccagaaccagggcatcatcgacaagaccgtgagcagcctgagactgaccaaggacgaggaggagaaggaccacgtgtaccagaacgccgccgagctggtgtggggcatggtgagcaactgcctgaccccctacttcaacgaccccaagaacaagtacatcctgaagtacaaggacgccaagacccccggcgacttcgaggactggatcaccagcaagatcagcgaggacgacggcgagcccttcgtgaaggtgctgagcttcctgtgcaacttcctggagggcaaggagatcaacgagctgctgaccgcctacatccacaagttcgagtgcatccaggacttcctgaacgtgatcagcagcctgggcgagaacgtgcagttccagcccagattcgccctgttcaacaacgccagcttcgcccagaacgtggccgtgcagctgagaatcctggccagcatcggcaagatgaagcccgacctgaccgaggccaagaggcccctgtacaaggccgccatcagaatgctgtgcccccccgagaagtgggagaagtacaccagcgacgagtggctggagaagaacatgctgctgaacagcgaggacagaaagaacgacaagaagaagaagcaggtgaaccccttcagaaacttcatcgccggcaacgtgatcgagagcagaagattcatgtacctggtgagatacagcaagcccaaggccgtgagagccatcatgcagaacagaagcatcgtgaactacgtgctgcacagactgcccagcgagcaggtgcacagatacgccagcgtgttccccgagaacttcgccgacctggagcaggagatcgacttcctgaccaagaagctgttcgagttcagcttcgaggagctgctgcacgagaaggacgtgatcctgaacaacagcagaagccacaagcccagcctggagatcgagagactgaaggccatcaccggcctgtacctgagcgtggcctacatcgccatcaagaacatcgtgaaggccaacgccagatactacatcgccttcgccgtgttcgagagagacaaggagctggtgaaggccaaggacgccagaatccagaccaagatccccgagacagacttccccgactacttctgcctgacccagtactacctggacagagacgaggagaagaagttccccggcgaccccagagacaaggaggccttcttcgagcacctgagaaagaccaagagacacttcagcaagcagtggagagagtggctgaacgagaagatcgccgacgccaagagcagccaggccaccggcctgctgctgagagaggccagaaacgacgtggagcacctgaacgtgctgagagccatccccgactacatccaggacttcagacacggcgagaagggcgagacagccatgaacagctacttcgagctgtaccactacctgatgcagagactgatgctgaagaacaccgagctggacctgagccactggagcggctggatcatgagaagcggcagacccgacagagacttgatccagatcgccttcgtgagcctggcctacaacctgcccagatacagaaacctgaccaaggagcaccacttcgacgacaccgtgctgcagaagatcagagagaaggagagcctggacacaggcggcggccccggcggcggcgccgccgccggcagcggcagccctaagaaaaaacgaaaagttggcagcggaagcaaaaggccggcggccacgaaaaaggccggccaggcaaaaaagaaaaagtaactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 11 - protein, 982 aa, synthetic construct
MGSSHHHHHHSSGLVPRGSHMASMTGGQQMGRGSPAAKKKKLDGSVDMSKDKKTKAKRMGVKALLAHGEDKLTMTTFGKGNRSKIEFTEGYHGRALETPKHFGIRGFEVRRIDENVDLCGDLEEGKTIEALLVNPSEKVGEDYLKLKGTLEKRFFGREFPHDNIRIQLIYNILDIYKILGMNVADILYALGNMQDTELDIDMFGQSLNNEDNLKECLKRMRPYMGYFGDIFKISPKGENIADREHNKKVLRCISVLRNATAHDKQDEYPWFKSSDIYETKIFKADMWKIIKDQYREKIKKVNKDFLSKNAVNMAILFDLLNARDVEQKKQITDEFYRFTIRKDGKNLGMNLVKIREIIIDRYASGLRDKKHDPHRQKINVIADFLIFRALSQNQGIIDKTVSSLRLTKDEEEKDHVYQNAAELVWGMVSNCLTPYFNDPKNKYILKYKDAKTPGDFEDWITSKISEDDGEPFVKVLSFLCNFLEGKEINELLTAYIHKFECIQDFLNVISSLGENVQFQPRFALFNNASFAQNVAVQLRILASIGKMKPDLTEAKRPLYKAAIRMLCPPEKWEKYTSDEWLEKNMLLNSEDRKNDKKKKQVNPFRNFIAGNVIESRRFMYLVRYSKPKAVRAIMQNRSIVNYVLHRLPSEQVHRYASVFPENFADLEQEIDFLTKKLFEFSFEELLHEKDVILNNSRSHKPSLEIERLKAITGLYLSVAYIAIKNIVKANARYYIAFAVFERDKELVKAKDARIQTKIPETDFPDYFCLTQYYLDRDEEKKFPGDPRDKEAFFEHLRKTKRHFSKQWREWLNEKIADAKSSQATGLLLREARNDVEHLNVLRAIPDYIQDFRHGEKGETAMNSYFELYHYLMQRLMLKNTELDLSHWSGWIMRSGRPDRDLIQIAFVSLAYNLPRYRNLTKEHHFDDTVLQKIREKESLDTGGGPGGGAAAGSGSPKKKRKVGSGSKRPAATKKAGQAKKKK

SEQ ID NO: 12 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 13 - DNA, 4828 bp, synthetic construct
  source 1..4828: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 14 - DNA, 6803 bp, synthetic construct
  source 1..6803: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgccggcagctaagaaaaagaaactggatggcagcgtcgacatgagcaaggacaagaaaaccaaggccaagagaatgggcgtgaaggccctgctggcccacggcgaggacaagctgaccatgaccaccttcggcaagggcaacagaagcaagatcgagttcaccgagggctaccacggcagagccctggagacacccaagcacttcggcatcagaggcttcgaggtgagaagaatcgacgagaacgtggacctgtgcggcgacctggaggagggcaagaccatcgaggccctgctggtgaaccccagcgagaaggtgggcgaggactacctgaagctgaagggcaccctggagaagagattcttcggcagagagttcccccacgacaacatcagaatccagctgatctacaacatcctggacatctacaagatcctgggcatgaacgtggccgacatcctgtacgccctgggcaacatgcaggacaccgagctggacatcgacatgttcggccagagcctgaacaacgaggacaacctgaaggagtgcctgaagagaatgaggccctacatgggctacttcggcgacatcttcaagatcagccccaagggcgagaacatcgccgacagagagcacaacaagaaggtgctgagatgcatcagcgtgctgagaaacgccaccgcccacgacaagcaggacgagtacccctggttcaagagcagcgacatctacgagacaaagatcttcaaggccgacatgtggaagatcatcaaggaccagtacagagagaagatcaagaaggtgaacaaggacttcctgagcaagaacgccgtgaacatggccatcctgttcgacctgctgaacgccagagacgtggagcagaagaagcagatcaccgacgagttctacagattcaccatcagaaaggacggcaagaacctgggcatgaacctggtgaagatcagagagatcatcatcgacagatacgccagcggcctgagagacaagaagcacgacccccacagacagaagatcaacgtgatcgccgacttcctgatcttcagagccctgagccagaaccagggcatcatcgacaagaccgtgagcagcctgagactgaccaaggacgaggaggagaaggaccacgtgtaccagaacgccgccgagctggtgtggggcatggtgagcaactgcctgaccccctacttcaacgaccccaagaacaagtacatcctgaagtacaaggacgccaagacccccggcgacttcgaggactggatcaccagcaagatcagcgaggacgacggcgagcccttcgtgaaggtgctgagcttcctgtgcaacttcctggagggcaaggagatcaacgagctgctgaccgcctacatccacaagttcgagtgcatccaggacttcctgaacgtgatcagcagcctgggcgagaacgtgcagttccagcccagattcgccctgttcaacaacgccagcttcgcccagaacgtggccgtgcagctgagaatcctggccagcatcggcaagatgaagcccgacctgaccgaggccaagaggcccctgtacaaggccgccatcagaatgctgtgcccccccgagaagtgggagaagtacaccagcgacgagtggctggagaagaacatgctgctgaacagcgaggacagaaagaacgacaagaagaagaagcaggtgaaccccttcagaaacttcatcgccggcaacgtgatcgagagcagaagattcatgtacctggtgagatacagcaagcccaaggccgtgagagccatcatgcagaacagaagcatcgtgaactacgtgctgcacagactgcccagcgagcaggtgcacagatacgccagcgtgttccccgagaacttcgccgacctggagcaggagatcgacttcctgaccaagaagctgttcgagttcagcttcgaggagctgctgcacgagaaggacgtgatcctgaacaacagcagaagccacaagcccagcctggagatcgagagactgaaggccatcaccggcctgtacctgagcgtggcctacatcgccatcaagaacatcgtgaaggccaacgccagatactacatcgccttcgccgtgttcgagagagacaaggagctggtgaaggccaaggacgccagaatccagaccaagatccccgagacagacttccccgactacttctgcctgacccagtactacctggacagagacgaggagaagaagttccccggcgaccccagagacaaggaggccttcttcgagcacctgagaaagaccaagagacacttcagcaagcagtggagagagtggctgaacgagaagatcgccgacgccaagagcagccaggccaccggcctgctgctgagagaggccagaaacgacgtggagcacctgaacgtgctgagagccatccccgactacatccaggacttcagacacggcgagaagggcgagacagccatgaacagctacttcgagctgtaccactacctgatgcagagactgatgctgaagaacaccgagctggacctgagccactggagcggctggatcatgagaagcggcagacccgacagagacttgatccagatcgccttcgtgagcctggcctacaacctgcccagatacagaaacctgaccaaggagcaccacttcgacgacaccgtgctgcagaagatcagagagaaggagagcctggacacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagataactctacaaacctgtagggttctgagactgccgttcttctgcttgtcggccatgatatttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 15 - DNA, 6793 bp, synthetic construct
  source 1..6793: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgccggcagctaagaaaaagaaactggatggcagcgtcgacatgagcaaggacaagaaaaccaaggccaagagaatgggcgtgaaggccctgctggcccacggcgaggacaagctgaccatgaccaccttcggcaagggcaacagaagcaagatcgagttcaccgagggctaccacggcagagccctggagacacccaagcacttcggcatcagaggcttcgaggtgagaagaatcgacgagaacgtggacctgtgcggcgacctggaggagggcaagaccatcgaggccctgctggtgaaccccagcgagaaggtgggcgaggactacctgaagctgaagggcaccctggagaagagattcttcggcagagagttcccccacgacaacatcagaatccagctgatctacaacatcctggacatctacaagatcctgggcatgaacgtggccgacatcctgtacgccctgggcaacatgcaggacaccgagctggacatcgacatgttcggccagagcctgaacaacgaggacaacctgaaggagtgcctgaagagaatgaggccctacatgggctacttcggcgacatcttcaagatcagccccaagggcgagaacatcgccgacagagagcacaacaagaaggtgctgagatgcatcagcgtgctgagaaacgccaccgcccacgacaagcaggacgagtacccctggttcaagagcagcgacatctacgagacaaagatcttcaaggccgacatgtggaagatcatcaaggaccagtacagagagaagatcaagaaggtgaacaaggacttcctgagcaagaacgccgtgaacatggccatcctgttcgacctgctgaacgccagagacgtggagcagaagaagcagatcaccgacgagttctacagattcaccatcagaaaggacggcaagaacctgggcatgaacctggtgaagatcagagagatcatcatcgacagatacgccagcggcctgagagacaagaagcacgacccccacagacagaagatcaacgtgatcgccgacttcctgatcttcagagccctgagccagaaccagggcatcatcgacaagaccgtgagcagcctgagactgaccaaggacgaggaggagaaggaccacgtgtaccagaacgccgccgagctggtgtggggcatggtgagcaactgcctgaccccctacttcaacgaccccaagaacaagtacatcctgaagtacaaggacgccaagacccccggcgacttcgaggactggatcaccagcaagatcagcgaggacgacggcgagcccttcgtgaaggtgctgagcttcctgtgcaacttcctggagggcaaggagatcaacgagctgctgaccgcctacatccacaagttcgagtgcatccaggacttcctgaacgtgatcagcagcctgggcgagaacgtgcagttccagcccagattcgccctgttcaacaacgccagcttcgcccagaacgtggccgtgcagctgagaatcctggccagcatcggcaagatgaagcccgacctgaccgaggccaagaggcccctgtacaaggccgccatcagaatgctgtgcccccccgagaagtgggagaagtacaccagcgacgagtggctggagaagaacatgctgctgaacagcgaggacagaaagaacgacaagaagaagaagcaggtgaaccccttcagaaacttcatcgccggcaacgtgatcgagagcagaagattcatgtacctggtgagatacagcaagcccaaggccgtgagagccatcatgcagaacagaagcatcgtgaactacgtgctgcacagactgcccagcgagcaggtgcacagatacgccagcgtgttccccgagaacttcgccgacctggagcaggagatcgacttcctgaccaagaagctgttcgagttcagcttcgaggagctgctgcacgagaaggacgtgatcctgaacaacagcagaagccacaagcccagcctggagatcgagagactgaaggccatcaccggcctgtacctgagcgtggcctacatcgccatcaagaacatcgtgaaggccaacgccagatactacatcgccttcgccgtgttcgagagagacaaggagctggtgaaggccaaggacgccagaatccagaccaagatccccgagacagacttccccgactacttctgcctgacccagtactacctggacagagacgaggagaagaagttccccggcgaccccagagacaaggaggccttcttcgagcacctgagaaagaccaagagacacttcagcaagcagtggagagagtggctgaacgagaagatcgccgacgccaagagcagccaggccaccggcctgctgctgagagaggccagaaacgacgtggagcacctgaacgtgctgagagccatccccgactacatccaggacttcagacacggcgagaagggcgagacagccatgaacagctacttcgagctgtaccactacctgatgcagagactgatgctgaagaacaccgagctggacctgagccactggagcggctggatcatgagaagcggcagacccgacagagacttgatccagatcgccttcgtgagcctggcctacaacctgcccagatacagaaacctgaccaaggagcaccacttcgacgacaccgtgctgcagaagatcagagagaaggagagcctggacacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagataactctacaaacctgtagggttctgagacggagaccacggcaggtctcattttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 16 - DNA, 9363 bp, synthetic construct
  source 1..9363: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaataaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagcgtcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcgccaccatggccagcgagttcaagaagaagctcttctggagggcagtggtggccgagttcctggccacgaccctctttgtcttcatcagcatcggttctgccctgggcttcaaatacccggtggggaacaaccagacggcggtccaggacaacgtgaaggtgtcgctggccttcgggctgagcatcgccacgctggcgcagagtgtgggccacatcagcggcgcccacctcaacccggctgtcacactggggctgctgctcagctgccagatcagcatcttccgtgccctcatgtacatcatcgcccagtgcgtgggggccatcgtcgccaccgccatcctctcaggcatcacctcctccctgactgggaactcgcttggccgcaatgacctggctgatggtgtgaactcgggccagggcctgggcatcgagatcatcgggaccctccagctggtgctatgcgtgctggctactaccgaccggaggcgccgtgaccttggtggctcagccccccttgccatcggcctctctgtagcccttggacacctcctggctattgactacactggctgtgggattaaccctgctcggtcctttggctccgcggtgatcacacacaacttcagcaaccactggattttctgggtggggccattcatcgggggagccctggctgtactcatctacgacttcatcctggccccacgcagcagtgacctcacagaccgcgtgaaggtgtggaccagcggccaggtggaggagtatgacctggatgccgacgacatcaactccagggtggagatgaagcccaaatacccatacgatgttccagattacgctggatccgctagcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcctaggcggccgcgcatgccctgcaggtgatctatcgatcggccggcccctctccctccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccgggccggatatcacgcgtgatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcaatgcatacatgtgtttaaacctcgacttaattaagtcgagggtcgacggtatcgataagctcgcttcacgagatcatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 17 - DNA, 7110 bp, synthetic construct
  source 1..7110: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgagccccaagaagaagagaaaggtggaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggacctaagaaaaagaggaaggtggcggccgcttacccatacgatgttccagattacgcttgaggtaccctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaagtaaacccctaccaactggtcggggtttgaaacagggcagaaccgatgctgatgaagaccaagtaaacccctaccaactggtcggggtttgaaactttttttcccgggaatggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 18 - DNA, 7114 bp, synthetic construct
  source 1..7114: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgagccccaagaagaagagaaaggtggaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggacctaagaaaaagaggaaggtggcggccgcttacccatacgatgttccagattacgcttgaggtaccctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaagtaaacccctaccaactggtcggggtttgaaacgtggttggagaactggatgtagatgggctgcaagtaaacccctaccaactggtcggggtttgaaactttttttcccgggaatggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 19 - DNA, 4780 bp, synthetic construct
  source 1..4780: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccacgaccctctttgtcttcactcgagtgaagacaaagagggtcgtggttttttggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 20 - DNA, 4780 bp, synthetic construct
  source 1..4780: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcagcccatctacatccagttcctcgaggaactggatgtagatgggctgttttttggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 21 - DNA, 7108 bp, synthetic construct
  source 1..7108: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgagccccaagaagaagagaaaggtggaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggacctaagaaaaagaggaaggtggcggccgcttacccatacgatgttccagattacgcttgaggtaccctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaagtaaacccctaccaactggtcggggtttgaaacgatcaacattaaatgtgagcgagtcaagtaaacccctaccaactggtcggggtttgaaactttttttcccgggaatggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 22 - DNA, 34 bp, synthetic construct
  source 1..34: mol_type = other DNA
agacgtggttggagaactggatgtagatgggctg

SEQ ID NO: 23 - DNA, 34 bp, synthetic construct
  source 1..34: mol_type = other DNA
aaaacagcccatctacatccagttctccaaccac

SEQ ID NO: 24 - DNA, 30 bp, synthetic construct
  source 1..30: mol_type = other DNA
agacagggcagaaccgatgctgatgaagac

SEQ ID NO: 25 - DNA, 30 bp, synthetic construct
  source 1..30: mol_type = other DNA
aaaagtcttcatcagcatcggttctgccct

SEQ ID NO: 26 - DNA, 22 bp, synthetic construct
  source 1..22: mol_type = other DNA
attgtcccagatatagccgttg

SEQ ID NO: 27 - DNA, 20 bp, synthetic construct
  source 1..20: mol_type = other DNA
gctgtcatttccgtttgctg

SEQ ID NO: 28 - DNA, 20 bp, synthetic construct
  source 1..20: mol_type = other DNA
gctcttctggagggcagtgg

SEQ ID NO: 29 - DNA, 21 bp, synthetic construct
  source 1..21: mol_type = other DNA
cagtgtgacagccgggttgag

SEQ ID NO: 30 - DNA, 20 bp, synthetic construct
  source 1..20: mol_type = other DNA
ccatggggaaggtgaaggtc

SEQ ID NO: 31 - DNA, 21 bp, synthetic construct
  source 1..21: mol_type = other DNA
gaaggggtcattgatggcaac

SEQ ID NO: 32 - protein, 928 aa, synthetic construct
MSKDKKTKAKRMGVKALLAHGEDKLTMTTFGKGNRSKIEFTEGYHGRALETPKHFGKRGFEVRKIDENVDLYGDLEEGKTIEALLINPSEKVGEDYLKLKGTLEKRFFGREFPHDNIRIQLIYNILDIYKILGMNVADILYALGNMQDTELDIDMFGQSLNNPEKAKDVVQRMKPYMGFFGGIFRTQKKEDRKKDSNLSEEEKEEKKKKQEELLQKDLQHNMDVARCISALRHATAHNKPATAHDKQDEYPWFKSSDIYETKIFKAGMWSVIEADYKKKIQDVNKQFFSKNKVNLAILFDLLDVRDVKQKKRISDEFYRFTIRKDGKNLGMNLVKIREIIIDRYASGLRDKKHDPHRQKINVIADFLIFRALSQNQGIIDKTVSSLRLTKDEEEKDHVYQNAAELVWGMVSNCLTPYFNDPKNKYILKYKDAKTPGDFEDWITSKISEDDGEPFVKVLSFLCNFLEGKEINELLTAYIHKFECIQDFLNVISSLGENVQFQPRFALFNNASFAQNVAVQLRILASIGKMKPDLTEAKRPLYKAAIRMLCPPEKWEKYTSDEWLEKNMLLNSEDRKNDKKKKQVNPFRNFIAGNVIESRRFMYLVRYSKPKAVRAIMQNRSIVNYVLHRLPSEQIKTYSRVFPEDFSDTEAEIDFLVNKLSEFSFETFISNQQTILNNSKRGFSPNRRETAEEIERLKAITGLYLSVAYIAIKNIVKANARYYIAFAVFERDKELVKAKDARIQTTIPNTKFTNYFCLTQYYLDRDEEKKFPGDPRDKEAFFEHLRKTKRHFSKQWREWLNEKIADAKSSQATGLLLREARNDVEHLNVLRAIPDYIQDFRHGEKGETAMNSYFELYHYLMQRLMLKNTELDLSHWSGWIMRSGRPDRDLIQIAFVSLAYNLPRYRNLTKEHHFDDTVLQKIREKESLD

SEQ ID NO: 33 - RNA, 36 nt, synthetic construct
  source 1..36: mol_type = other RNA
ggtcaacagtgtggggaattacccgcactctacaac

SEQ ID NO: 34 - protein, 891 aa, synthetic construct
MSKDKKTKAKRMGVKALLAHGEDKLTMTTFGKGNRSQIEFTEGYHGRALETPKRFGKRGFEVRKIDENVDLYGDLDEGKTIEALLVNPSENVGEDYLKLKSTLENYFFGREFPHDNIRIQLIYNILDIYKILGMNVADILYTLGNLQDAEGDIDLFGKSLNNEDNIKESLNRMRPYMGYFGEVFKKDNREHNKKVLRCISALRNATAHGKQDEYPWFKSSDIYEKTIFKADKWRIIEDQYREKIRKVNNEFFSKNKVNLAILFDLLHARDVEPKKQIADEFYRFTIRKDGKNLGMNLVKIREKIIDRFARDLRDKKHDPHRQKIHVIADFLIFRALSQNQEVIDKTVSRLRLTKDEEEKDRVYQNAAELVWGMVSNCLSPYFKDPKKYIIQYKSGGKIKIFDDWITSKISAKDGEPFVKVLSFLCNFLEGKEINELLTAYIHKFECIQDFLKVISSLGERAQFQPRFDLFNKPDFAQNVAVQLRILASIGKMKPDLTEAKRPLYKAAIQMLCPPEKWEKYTSDEWLEENMLLNSEDRQNKEKREKVNPFRNFIAGNVIESRRFMYLVRYSKPKAVRALMKNRSVVNYVLHRLPPEQIKTYSRVFPDDFNDTEAEIDFLVNKLSEFSFETFITSRQTILANSKRGFSPNRRETAEEIERLKAITGLYLSVAYIAIKNIVKANARYYIAFAVFERDKELVKAKDARIQTTIPNTTYTNYFCLTQYYLDRDEEKKFQGDPRDKEAFFEHLRKKKTHFSKQWREWLNEKIADAKSAQETGLLLTEARNDVEHLNVLRAIPDYIQDFRHGDKGETPMNSYFELYHYLLQRLMLKNRLLDLSSWRSWIERSGRPDRDLIQIAFVSLAYNLPRYRNLTKEHHFDDTVLQKIRERKSLD

SEQ ID NO: 35 - RNA, 34 nt, synthetic construct
  source 1..34: mol_type = other RNA
agtctcagaaccctacaggtttgtagagttatct

SEQ ID NO: 36 - DNA, 6898 bp, synthetic construct
  source 1..6898: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgccggcagctaagaaaaagaaactggatggcagcgtcgacatgagcaaggataagaaaaccaaagccaagaggatgggtgtgaaagccctgctggcccacggagaggacaagctgacaatgaccacattcggaaagggcaatagatccaaaatcgagttcacagagggataccacggcagggccctggaaacccccaagcactttggcaagaggggattcgaggtccggaagatcgatgagaatgttgacctgtacggggacctcgaggagggcaaaaccattgaagccctgctcattaatcctagcgagaaggtgggcgaggactacctcaagctgaaggggactctcgagaaacggttttttgggcgcgaatttccacatgacaatatcagaatccagctgatctacaacatcctggacatctacaaaatactgggcatgaacgtcgccgacatcctgtacgctctgggcaatatgcaggacacagagctggacattgatatgttcggccagtctctgaataatcctgagaaggccaaagatgtggtgcagaggatgaagccttacatgggcttttttggcggcatcttcagaacccagaagaaggaggaccggaagaaggacagcaatctcagcgaggaggagaaggaggagaagaagaagaagcaggaagagctgctgcagaaggacctgcagcacaacatggatgtcgccagatgtattagcgcactgagacatgccaccgcccacaataagccagccactgcccacgataagcaggacgaatacccctggttcaagagcagcgacatttatgagacaaagattttcaaagcaggcatgtggtcagtgatcgaggctgattataagaagaagatccaggatgttaataaacagttcttcagcaagaataaggtgaacctggccatcctctttgacctgctggacgtacgtgatgtgaagcaaaaaaagcggatcagcgatgagttctaccgttttaccatcaggaaggacgggaaaaacctgggcatgaatctcgtgaaaattagagagatcattatcgaccgctacgccagcgggctgcgcgataagaaacatgatccccacagacagaaaatcaacgtaattgcagacttcctgattttccgggccctgtcacagaaccaggggatcatagataagactgtgtctagtctgcggctgaccaaggacgaggaggagaaggaccatgtgtatcagaatgcagccgaactggtgtgggggatggtgtccaactgcctgaccccatacttcaatgatcccaagaataaatacatcctgaaatacaaagacgccaagactcccggggatttcgaggattggatcaccagcaaaatcagtgaggacgatggggagccttttgtgaaggtgctgagctttctgtgtaatttcttagagggaaaggaaattaacgagctgcttaccgcctacatccataaattcgaatgtatccaggattttctgaacgtgatatcttcactgggcgagaacgtccagtttcagcccagatttgccctgtttaataatgctagcttcgctcagaacgttgccgtccagctgaggattttggccagcatcggcaagatgaagcctgatctgaccgaggccaaaaggcccctgtacaaagcagccatcagaatgctgtgcccccccgagaagtgggagaagtacaccagtgatgaatggctggagaagaacatgctgctgaatagcgaggacagaaagaacgacaagaaaaagaaacaggtgaaccctttcaggaattttattgcagggaacgttatcgagtctcgccgctttatgtatctggtgaggtacagcaagccaaaggccgtgcgggccatcatgcagaacaggtcaatcgtgaactacgtgctccaccggctgccttcagagcagatcaaaacttactcccgtgtgttccccgaggacttctccgacaccgaggccgaaatcgactttctcgtgaataaactgagtgaattttcatttgaaaccttcatctccaaccagcagacaattctgaacaactccaagagaggctttagccccaatagacgggaaacggccgaggagattgaacggctgaaggccattaccggcctgtacctctcagtggcctatatcgccatcaaaaacatcgtgaaggccaacgctaggtactatattgcattcgccgtgttcgaaagagataaggagctggtgaaagccaaggacgcccgcatacagaccactatccctaacacaaaatttactaactatttttgtctgactcagtactatctggaccgcgacgaagagaagaagtttcctggcgacccccgcgacaaggaggccttctttgaacatctgaggaaaactaagcggcactttagtaaacagtggagagagtggctgaacgagaagatcgccgacgccaagagcagccaggccaccgggctgctgctgcgggaggcaagaaatgatgtggaacacctgaatgtgctgagagctatccccgactacatccaggactttcgccacggagagaagggcgagacagccatgaatagctacttcgagctgtatcactacctgatgcagaggctgatgcttaagaacaccgagctggacctgagccactggagcggctggatcatgaggtccggtcgacctgacagggatctgatacagattgctttcgtgtccctggcatacaatctgccacggtacagaaatctgaccaaagagcatcacttcgatgacaccgtgcttcagaagatcagggagaaagagagcctggatacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtcaacagtgtggggaattacccgcactctacaacggagaccacggcaggtctcattttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 37 - DNA, 6786 bp, synthetic construct
  source 1..6786: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgccggcagctaagaaaaagaaactggatggcagcgtcgacatgtccaaggacaagaaaaccaaggccaagcggatgggcgtgaaagcactgctggcccatggcgaggataagctcaccatgaccacttttggtaagggaaataggtcccagatcgagtttacagaaggctatcacggccgagccctggagacacccaaaagattcggcaagagggggtttgaggtgagaaagatcgatgaaaacgtcgacctgtatggtgacctggacgagggcaagactattgaggccctgctggtgaacccctccgagaacgttggagaggactatctgaaactgaaaagcacactggagaactattttttcggtcgcgagttccctcatgacaatatccgcatccagctgatctacaacatcttggatatctataagatcctggggatgaacgtggctgacatcctctacaccctgggcaatctgcaggacgctgaaggagatatcgatctgttcgggaagtccctcaacaacgaggacaacattaaagaaagtctgaatagaatgaggccctacatgggctactttggggaggtgtttaaaaaggacaatagagaacataacaagaaagtgctccggtgcatctccgctctgcgcaacgccacagcccacggcaaacaggatgaatatccttggttcaagagcagcgatatctatgaaaagactatcttcaaagccgacaagtggcggatcatcgaggaccagtacagagaaaagattaggaaggtgaacaacgagtttttcagcaaaaacaaagtcaatctggctatcctcttcgatcttctgcacgccagagatgtggaacctaagaaacagattgccgatgaattttacaggttcaccatcagaaaggatggcaaaaatctggggatgaacctggtgaagatcagggagaaaatcatcgatcggtttgcccgcgatctgcgagacaagaaacacgacccacacagacagaaaattcacgtgatcgccgatttcctgattttcagggccctgagtcagaatcaggaagtgatcgacaaaacagtcagccggctgaggctgaccaaggatgaagaggagaaagatagagtgtaccagaacgccgccgagctcgtgtggggaatggtgtccaactgtctgagcccatatttcaaagaccctaagaaatacatcatccagtataagtccggcggaaaaatcaaaatcttcgacgactggatcacctctaagatttccgccaaagatggcgagccatttgtgaaggtgctgtcctttctctgcaatttcctggagggcaaggagatcaacgagctgctgaccgcctacatccacaagttcgagtgcatccaggattttctgaaagtcatttccagcctgggcgagagggcccagtttcagcctcggttcgatctgttcaataagcctgactttgcacagaacgtggccgtgcagctgcgcatcctcgccagtattgggaaaatgaagcccgatctgactgaggccaaaaggcctctctacaaagctgccatccagatgctctgccctcctgagaaatgggagaaatacacaagcgacgagtggctggaggagaacatgctgctgaactccgaggataggcagaataaggagaagcgagagaaggtcaatccctttcggaatttcatcgccggcaatgtgattgagagccgccgatttatgtacctcgtgaggtactccaagcccaaggcagttcgggctctgatgaaaaatagatcagtggtgaactacgtgctgcacaggctgcctccagagcagatcaagacctactccagggtgttccctgatgacttcaatgatactgaagctgagatcgatttcctggtgaacaagctcagcgaattttccttcgagacattcatcaccagcagacagaccattctggcaaattccaagagggggttttctcctaaccggcgggagacagccgaggagatcgaaaggctgaaagccatcacaggcctgtacctcagcgtggcctacattgctatcaaaaatatcgtgaaagcaaatgcccgatactacatagccttcgccgtgttcgagcgggacaaggagctcgtgaaagccaaggacgccaggatacagactaccatccccaatacaacctacactaattacttctgcctgactcagtactacctggaccgtgacgaggagaaaaagtttcagggcgaccctcgggataaggaggctttctttgaacatctccgtaagaagaaaacacactttagcaagcagtggcgggagtggctgaatgagaagatcgctgatgccaagtccgcccaggaaaccggcctgctgctgaccgaggccaggaatgacgtggaacacctgaatgtgctgcgggccattcccgattatatccaggacttcaggcacggagataagggggaaactcctatgaacagctacttcgagctgtatcattacctgctgcagaggctcatgctgaaaaatcggctgctcgacctgtctagctggcgctcctggatcgagaggtctggccgacctgatcgcgacctgattcagatcgcatttgtgagcctggcttataatctgcctagatatcggaatctgaccaaggagcaccactttgatgataccgtgctgcagaagattagagaaaggaagagcctggacacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagtctcagaaccctacaggtttgtagagttatctggagaccacggcaggtctcattttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 38 - DNA, 34 bp, synthetic construct
  source 1..34: mol_type = other DNA
caacgtggttggagaactggatgtagatgggctg

SEQ ID NO: 39 - DNA, 34 bp, synthetic construct
  source 1..34: mol_type = other DNA
atctgtggttggagaactggatgtagatgggctg

SEQ ID NO: 40 - RNA, 21 nt, synthetic construct
  source 1..21: mol_type = other RNA
gcccatctacatccagttctc

SEQ ID NO: 41 - RNA, 21 nt, synthetic construct
  source 1..21: mol_type = other RNA
cagcccatctacatccagttc

SEQ ID NO: 42 - RNA, 30 nt, synthetic construct
  source 1..30: mol_type = other RNA
catgaaaaacttgagagttgctgggtctga

SEQ ID NO: 43 - RNA, 30 nt, synthetic construct
  source 1..30: mol_type = other RNA
gaattaagttagttagttgctcttctaaat

SEQ ID NO: 44 - RNA, 30 nt, synthetic construct
  source 1..30: mol_type = other RNA
cgatgttgaattaatgtccatggactacct

SEQ ID NO: 45 - RNA, 30 nt, synthetic construct
  source 1..30: mol_type = other RNA
gatagagaaatttctgtgggttcttgaata

SEQ ID NO: 46 - RNA, 30 nt, synthetic construct
  source 1..30: mol_type = other RNA
ctggagaaggtctttgatgctattatcttg

SEQ ID NO: 47 - RNA, 30 nt, synthetic construct
  source 1..30: mol_type = other RNA
cactatggagtatatcttctctaggcccaa

SEQ ID NO: 48 - RNA, 30 nt, synthetic construct
  source 1..30: mol_type = other RNA
ccacactcatcatgccaccaccagcctcct

SEQ ID NO: 49 - RNA, 30 nt, synthetic construct
  source 1..30: mol_type = other RNA
gaccatctaaaattgattcccacatcacaa

SEQ ID NO: 50 - DNA, 21 bp, synthetic construct
  source 1..21: mol_type = other DNA
ccagaacacccagaagtaact

SEQ ID NO: 51 - DNA, 24 bp, synthetic construct
  source 1..24: mol_type = other DNA
tctgtgggttcttgaatactagtc

SEQ ID NO: 52 - DNA, 9915 bp, synthetic construct
  source 1..9915: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaataaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagcgtcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcgccaccatggccatgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgatctatcgctgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgaaactccagaacacccagaagtaacttcacttaaaacttttgtagaaaaacaagataatagcatcaaagaccttctccagaccgtggaagaccaatataaacaattaaaccaacagcatagtcaaataaaagaaatagaaaatcagctcagaaggactagtattcaagaacccacagaaatttctctatcttccaagccaagagcaccaagaactactccctttcttcagttgaatgaaataagaaatgtaaaacatgatggcattcctgctgaatgtaccaccatttataacagaggtgaacatacaagtggcatgtatgccatcagacccagcaactctcaagtttttcatgtctactgtgatgttatatcaggtagtccatggacattaattcaacatcgaatagatggatcacaaaacttcaatgaaacgtgggagaactacaaatatggttttgggaggcttgatggagaattttggttgggcctagagaagatatactccatagtgaagcaatctaattatgttttacgaattgagttggaagactggaaagacaacaaacattatattgaatattctttttacttgggaaatcacgaaaccaactatacgctacatctagttgcgattactggcaatgtccccaatgcaatcccggaaaacaaagatttggtgttttctacttgggatcacaaagcaaaaggacacttcaactgtccagagggttattcaggaggctggtggtggcatgatgagtgtggagaaaacaacctaaatggtaaatataacaaaccaagagcaaaatctaagccagagaggagaagaggattatcttggaagtctcaaaatggaaggttatactctataaaatcaaccaaaatgttgatccatccaacagattcagaaagctttgaaggatccgctagcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcctaggcggccgcgcatgccctgcaggtgatctatcgatcggccggcccctctccctccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccgggccggatatcacgcgtgatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcaatgcatacatgtgtttaaacctcgacttaattaagtcgagggtcgacggtatcgataagctcgcttcacgagatcatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 53 - protein, 11 aa, synthetic construct
SPKKKRKVEAS

SEQ ID NO: 54 - protein, 11 aa, synthetic construct
GPKKKRKVAAA

SEQ ID NO: 55 - protein, 7 aa, synthetic construct
PKKKRKV

SEQ ID NO: 56 - protein, 16 aa, synthetic construct
KRPAATKKAGQAKKKK

SEQ ID NO: 57 - protein, 9 aa, synthetic construct
PAAKRVKLD

SEQ ID NO: 58 - protein, 11 aa, synthetic construct
RQRRNELKRSP

SEQ ID NO: 59 - protein, 38 aa, synthetic construct
NQSSNFGPMKGGNFGGRSSGPYGGGGQYFAKPRNQGGY

SEQ ID NO: 60 - protein, 42 aa, synthetic construct
RMRIZFKNKGKDTAELRRRRVEVSVELRKAKKDEQILKRRNV

SEQ ID NO: 61 - protein, 8 aa, synthetic construct
VSRKRPRP

SEQ ID NO: 62 - protein, 8 aa, synthetic construct
PPKKARED

SEQ ID NO: 63 - protein, 8 aa, synthetic construct
POPKKKPL

SEQ ID NO: 64 - protein, 12 aa, synthetic construct
SALIKKKKKMAP

SEQ ID NO: 65 - protein, 5 aa, synthetic construct
DRLRR

SEQ ID NO: 66 - protein, 7 aa, synthetic construct
PKQKKRK

SEQ ID NO: 67 - protein, 10 aa, synthetic construct
RKLKKKIKKL

SEQ ID NO: 68 - protein, 10 aa, synthetic construct
REKKKFLKRR

SEQ ID NO: 69 - protein, 20 aa, synthetic construct
KRKGDEVDGVDEVAKKKSKK

SEQ ID NO: 70 - protein, 17 aa, synthetic construct
RKCLQAGMNLEARKTKK

SEQ ID NO: 71 - protein, 9 aa, synthetic construct
PAAKKKKLD

SEQ ID NO: 72 - DNA, 6802 bp, synthetic construct
  source 1..6802: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgccggcagctaagaaaaagaaactggatggcagcgtcgacatgagcaaggacaagaaaaccaaggccaagagaatgggcgtgaaggccctgctggcccacggcgaggacaagctgaccatgaccaccttcggcaagggcaacagaagcaagatcgagttcaccgagggctaccacggcagagccctggagacacccaagcacttcggcatcagaggcttcgaggtgagaagaatcgacgagaacgtggacctgtgcggcgacctggaggagggcaagaccatcgaggccctgctggtgaaccccagcgagaaggtgggcgaggactacctgaagctgaagggcaccctggagaagagattcttcggcagagagttcccccacgacaacatcagaatccagctgatctacaacatcctggacatctacaagatcctgggcatgaacgtggccgacatcctgtacgccctgggcaacatgcaggacaccgagctggacatcgacatgttcggccagagcctgaacaacgaggacaacctgaaggagtgcctgaagagaatgaggccctacatgggctacttcggcgacatcttcaagatcagccccaagggcgagaacatcgccgacagagagcacaacaagaaggtgctgagatgcatcagcgtgctgagaaacgccaccgcccacgacaagcaggacgagtacccctggttcaagagcagcgacatctacgagacaaagatcttcaaggccgacatgtggaagatcatcaaggaccagtacagagagaagatcaagaaggtgaacaaggacttcctgagcaagaacgccgtgaacatggccatcctgttcgacctgctgaacgccagagacgtggagcagaagaagcagatcaccgacgagttctacagattcaccatcagaaaggacggcaagaacctgggcatgaacctggtgaagatcagagagatcatcatcgacagatacgccagcggcctgagagacaagaagcacgacccccacagacagaagatcaacgtgatcgccgacttcctgatcttcagagccctgagccagaaccagggcatcatcgacaagaccgtgagcagcctgagactgaccaaggacgaggaggagaaggaccacgtgtaccagaacgccgccgagctggtgtggggcatggtgagcaactgcctgaccccctacttcaacgaccccaagaacaagtacatcctgaagtacaaggacgccaagacccccggcgacttcgaggactggatcaccagcaagatcagcgaggacgacggcgagcccttcgtgaaggtgctgagcttcctgtgcaacttcctggagggcaaggagatcaacgagctgctgaccgcctacatccacaagttcgagtgcatccaggacttcctgaacgtgatcagcagcctgggcgagaacgtgcagttccagcccagattcgccctgttcaacaacgccagcttcgcccagaacgtggccgtgcagctgagaatcctggccagcatcggcaagatgaagcccgacctgaccgaggccaagaggcccctgtacaaggccgccatcagaatgctgtgcccccccgagaagtgggagaagtacaccagcgacgagtggctggagaagaacatgctgctgaacagcgaggacagaaagaacgacaagaagaagaagcaggtgaaccccttcagaaacttcatcgccggcaacgtgatcgagagcagaagattcatgtacctggtgagatacagcaagcccaaggccgtgagagccatcatgcagaacagaagcatcgtgaactacgtgctgcacagactgcccagcgagcaggtgcacagatacgccagcgtgttccccgagaacttcgccgacctggagcaggagatcgacttcctgaccaagaagctgttcgagttcagcttcgaggagctgctgcacgagaaggacgtgatcctgaacaacagcagaagccacaagcccagcctggagatcgagagactgaaggccatcaccggcctgtacctgagcgtggcctacatcgccatcaagaacatcgtgaaggccaacgccagatactacatcgccttcgccgtgttcgagagagacaaggagctggtgaaggccaaggacgccagaatccagaccaagatccccgagacagacttccccgactacttctgcctgacccagtactacctggacagagacgaggagaagaagttccccggcgaccccagagacaaggaggccttcttcgagcacctgagaaagaccaagagacacttcagcaagcagtggagagagtggctgaacgagaagatcgccgacgccaagagcagccaggccaccggcctgctgctgagagaggccagaaacgacgtggagcacctgaacgtgctgagagccatccccgactacatccaggacttcagacacggcgagaagggcgagacagccatgaacagctacttcgagctgtaccactacctgatgcagagactgatgctgaagaacaccgagctggacctgagccactggagcggctggatcatgagaagcggcagacccgacagagacttgatccagatcgccttcgtgagcctggcctacaacctgcccagatacagaaacctgaccaaggagcaccacttcgacgacaccgtgctgcagaagatcagagagaaggagagcctggacacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagataactctacaaacctgtagggttctgagactgggtgcagcctgggaccacttggcatggttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 73 - DNA, 29 bp, synthetic construct
  source 1..29: mol_type = other DNA
tgggtgcagcctgggaccacttggcatgg

SEQ ID NO: 74 - DNA, 18 bp, synthetic construct
  source 1..18: mol_type = other DNA
agaaacgccaccgcccac

SEQ ID NO: 75 - DNA, 18 bp, synthetic construct
  source 1..18: mol_type = other DNA
gcaaacgccaccgccgcc

SEQ ID NO: 76 - DNA, 18 bp, synthetic construct
  source 1..18: mol_type = other DNA
agaaagaccaagagacac

SEQ ID NO: 77 - DNA, 18 bp, synthetic construct
  source 1..18: mol_type = other DNA
gcaaagaccaagagagcc

SEQ ID NO: 78 - DNA, 18 bp, synthetic construct
  source 1..18: mol_type = other DNA
agaaacgacgtggagcac

SEQ ID NO: 79 - DNA, 18 bp, synthetic construct
  source 1..18: mol_type = other DNA
gcaaacgacgtggaggcc

SEQ ID NO: 80 - DNA, 36 bp, synthetic construct
  source 1..36: mol_type = other DNA
ccgcacagtccctacaggtttgtagagtcatcttcc

SEQ ID NO: 81 - DNA, 36 bp, synthetic construct
  source 1..36: mol_type = other DNA
ggaagatgactctacaaacctgtagggactgtgcgg

SEQ ID NO: 82 - DNA, 36 bp, synthetic construct
  source 1..36: mol_type = other DNA
ggtgtacagggtgcctggatttgacagggttacagc

SEQ ID NO: 83 - DNA, 36 bp, synthetic construct
  source 1..36: mol_type = other DNA
gctgtaaccctgtcaaatccaggcaccctgtacacc

SEQ ID NO: 84 - DNA, 36 bp, synthetic construct
  source 1..36: mol_type = other DNA
ggtgtacagggtgcctagatttgacagggttacagc

SEQ ID NO: 85 - DNA, 36 bp, synthetic construct
  source 1..36: mol_type = other DNA
gctgtaaccctgtcaaatctaggcaccctgtacacc

SEQ ID NO: 86 - DNA, 34 bp, synthetic construct
  source 1..34: mol_type = other DNA
ggaagaactctacaaacctgtagggttctgagac

SEQ ID NO: 87 - DNA, 36 bp, synthetic construct
  source 1..36: mol_type = other DNA
ggaagataactctacaaacctgtagagttctgagac

SEQ ID NO: 88 - DNA, 20 bp, synthetic construct
  source 1..20: mol_type = other DNA
acctccaccatgccaagtgg

SEQ ID NO: 89 - DNA, 20 bp, synthetic construct
  source 1..20: mol_type = other DNA
cagggtctcgattggatggc

SEQ ID NO: 90 - DNA, 30 bp, synthetic construct
  source 1..30: mol_type = other DNA
tgccgttcttctgcttgtcggccatgatat

SEQ ID NO: 91 - DNA, 7023 bp, synthetic construct
  source 1..7023: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgccggcagctaagaaaaagaaactggatggcagcgtcgacatcgagaagaaaaaatcctttgctaagggcatgggcgttaagagcactctggtgtcaggctctaaagtgtacatgaccacattcgccgagggctctgatgctcgcctggaaaaaattgtggaaggagactcaatcagatccgtgaacgagggggaggccttcagcgccgagatggccgacaagaatgccggatataagattggaaacgctaagttcagccatcccaagggatacgccgtggtcgccaacaatcctctgtacactggacctgtgcagcaggacatgttaggtctgaaggagactctggagaagaggtacttcggcgagtccgcagatggcaacgacaacatttgcattcaggtgatccataacatcctggacattgagaagatcctggccgagtatatcaccaacgcagcctacgctgtgaataatatatctggtctggataaggatatcattgggttcgggaaattttctaccgtgtacacctacgatgagtttaaagaccccgagcaccacagagccgccttcaacaacaacgacaagctgatcaatgccattaaggcacagtacgatgagttcgacaattttctggacaacccccggctgggctacttcggccaggcttttttttctaaggaaggccgcaattacatcatcaactacggcaacgagtgctatgacattctggcccttctgagcggcctgaggcattgggtggtgcataacaacgaagaggaatcccgcatctcaagaacatggctgtacaatctcgacaaaaacctggacaatgagtacatttccactctgaactatctgtacgacaggatcacgaatgagctgaccaattccttttccaagaatagcgccgccaatgtgaactacatcgccgaaaccctggggatcaatcccgccgagttcgccgagcagtattttcgattcagcatcatgaaagagcagaaaaatctgggctttaatatcaccaaactgagagaagtgatgctggacaggaaggatatgtctgagatcaggaagaaccacaaggtgttcgacagtatcagaaccaaggtgtatacaatgatggatttcgtgatctataggtactacattgaggaggacgccaaggtggccgccgccaacaaatccctgcctgataacgagaagtccctgtctgagaaggatatcttcgtcatcaacctgaggggcagcttcaatgatgaccagaaggacgctctttactacgacgaagccaaccggatctggcggaagctggaaaacatcatgcacaacatcaaggagttccgcgggaacaaaacacgggagtacaagaaaaaagacgctcccagactgcccagaatcctgcctgcaggcagggatgtgagcgcattctccaagctgatgtacgccctgacaatgttcctggatggcaaggaaatcaacgacctgctgaccactctgatcaacaagtttgataacattcagagctttctgaaagtgatgcccctgatcggagtgaatgcaaaattcgtcgaagaatacgctttcttcaaggattctgctaaaatcgccgacgagctgaggctgattaagtcctttgcaaggatgggggagccaatcgccgacgccagaagagccatgtatattgatgctatcagaatcctgggcaccaatctgtcctatgacgaactgaaggccctggctgacacattttctctggatgagaatggtaacaagctgaagaaggggaagcacggcatgcgaaactttattattaacaatgttatctctaataagaggttccattatctgattcgctacggcgaccccgcccatttgcacgagatcgccaagaatgaagcggtggtgaagttcgtcctgggacggatcgccgacatccagaaaaagcagggccagaatggcaaaaaccagatcgacagatattatgagacttgcatcggcaaggataagggtaaaagtgtctccgagaaagtggatgccctgactaagatcattacggggatgaattacgaccagtttgacaaaaagaggagcgtgatcgaggacaccggcagagagaatgccgagcgggagaagtttaagaagatcatcagcctgtatctgaccgtgatttaccatatcctgaagaatatcgtgaatatcaatgccagatacgtgatcggcttccactgcgtggaaagagacgcccagctctacaaagagaaaggctacgacatcaatctgaagaagctggaggaaaagggctttagcagcgtgacaaaactgtgtgcaggaatcgacgagactgcccctgacaagaggaaggacgtggagaaagagatggccgaacgcgccaaggagtctatcgactccctggagtccgccaaccccaagctgtacgccaactacatcaagtatagcgacgagaagaaggcagaagagttcacccggcagatcaatagagagaaggccaaaaccgcactgaatgcctatctgaggaatacaaagtggaacgtgattattagagaagatctgctgaggattgacaataagacctgtaccctattcaggaacaaggccgtgcatctggaggtggccaggtacgtgcatgcctatattaatgatatagctgaagtcaactcctatttccagctgtatcattacatcatgcagcgtattattatgaacgaaagatatgaaaagagctccggcaaagtgtctgagtacttcgatgccgtgaacgacgaaaaaaagtacaatgatcgcctgctgaagctgctgtgtgtgcccttcgggtactgtatccccagatttaagaatctgtccattgaagccctgttcgatagaaacgaagccgccaaattcgacaaggagaagaagaaggtgagcggcaatagtacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaagtaaacccctaccaactggtcggggtttgaaacgtggttggagaactggatgtagatgggctgttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 92 - DNA, 7394 bp, synthetic construct
  source 1..7394: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgccggcagctaagaaaaagaaactggatggcagcgtcgacatgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtggttggagaactggatgtagatgggctggttgtggaaggtccagttttgaggggctattacaacttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 93 - DNA, 6447 bp, synthetic construct
  source 1..6447: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgccggcagctaagaaaaagaaactggatggcagcgtcgacgcccaggtgtccaaacagaccagcaagaaaagggaactgagcatcgatgagtaccagggtgctagaaagtggtgcttcacaatcgcctttaacaaagctctggtcaatagggataaaaacgatggcctcttcgtggagtccctgctgagacacgagaagtacagcaaacacgactggtacgacgaggataccagagccctgatcaagtgctccactcaggccgccaatgcaaaggccgaggctctgagaaattatttcagccactaccggcactcccccgggtgtctgaccttcactgcagaggatgagcttaggacaattatggagcgcgcctacgagagggccatttttgagtgtcggcgaagagaaactgaggtgatcatcgagtttccatctctgtttgaaggggaccgcatcacaaccgccggagtggtattttttgtgagtttcttcgtggaacggagagtgctggaccggctgtacggggcagttagtggcctgaagaagaacgaggggcagtacaagctgacaaggaaagctctgagcatgtactgtctgaaagattccagatttacaaaggcatgggataagagagtgctcctctttcgcgatatcctggcccagctgggcagaatcccagctgaggcttacgagtactaccacggcgaacagggcgacaaaaagagggccaacgacaatgagggcaccaaccccaaaaggcacaaggataagtttattgagtttgcactgcactacctggaggcccagcactctgagatctgctttggcagaaggcacatcgtgagagaggaagctggcgccggcgatgagcacaagaagcacagaaccaaagggaaggtggtggtggacttctccaagaaggatgaggatcagtcttactacatcagtaagaacaacgtgattgtgaggatcgataagaatgccggccctcgctcctaccgcatggggctcaatgaactgaagtacctggtcctgctctctctgcaggggaagggcgatgacgctatcgccaagctgtataggtatcggcagcatgtggagaacattctggacgtggtgaaggtgactgataaagataaccacgtgttcctgccaaggtttgtgctcgaacagcacggaatcggccggaaggccttcaagcagagaattgacgggcgcgtgaaacatgtgcgcggggtgtgggaaaagaagaaggccgccacaaacgagatgacgctgcacgagaaggcccgcgacatcctccagtacgtgaatgagaattgcactcgcagcttcaatcctggcgagtataacaggctgctggtgtgcctggtgggtaaagacgtggagaacttccaggccggcctgaagcggcttcagctggctgagagaattgacggccgggtgtattctatcttcgcccagacttctaccatcaatgaaatgcaccaggtggtgtgtgaccagatcctcaatcggctgtgtcgaattggggatcagaagctgtacgactatgtgggcctgggcaagaaagatgagatcgactataaacagaaagtggcttggttcaaagagcacatcagtatcagacgcggctttctgaggaagaaattctggtacgactctaaaaagggcttcgctaagctggtggaggagcacctggaatccggaggcggacagcgggatgtggggctggataagaaatactaccacatcgatgccatcgggagattcgagggcgccaatcctgctctgtacgaaaccctggccagggacaggctgtgcctgatgatggcccagtactttctgggctccgttcgcaaagagctgggaaacaaaattgtgtggagcaacgactccatagagctgccggtggagggatctgtcgggaatgaaaaatcgatcgtgttctctgtgagcgattatggaaaactgtacgtcctggacgatgccgagtttctgggtagaatctgtgagtattttatgccacatgaaaagggcaagatcagataccacaccgtgtatgaaaaggggttcagagcctacaacgacctgcagaaaaaatgcgtggaggccgtgctggcattcgaagagaaggtggtgaaggccaagaaaatgtctgagaaagagggagcccactacatcgactttcgcgagatcctggcccagaccatgtgcaaggaagctgaaaagacagccgtcaataaggtgaggagggccttctttcaccaccaccttaaattcgtgatcgacgaatttggcctgttcagcgatgtgatgaaaaaatacggcatcgagaaggagtggaagtttccagtgaaaacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggttggagaactggatgtagatgggctggctggagcagcccccgatttgtggggtgattacagcttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 94 - DNA, 6492 bp, synthetic construct
  source 1..6492: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgccggcagctaagaaaaagaaactggatggcagcgtcgacaatggcatcgagctcaaaaaggaggaagccgcattttatttcaaccaggcagaactcaatctgaaggctattgaggataatatcttcgacaaggagagaagaaagaccctgctgaataaccctcagattctggccaagatggaaaattttatttttaactttagagacgtgaccaagaacgccaagggcgaaatcgactgcctgctcctgaagctgcgggagctgagaaatttctactcacattatgtgcacaaaagagatgtgagggaactgagcaagggggagaagccaattctggagaagtattatcagtttgccatcgagtctactggctctgagaacgtgaaactggagatcattgaaaacgacgcctggctggcagacgccggggtgctgttttttctgtgcatcttcctgaaaaagtcccaggcgaacaagttgatcagcggcatctctggttttaagagaaacgacgataccggccaacctcgcagaaacctgttcacttatttcagcatcagggaggggtacaaagtggtgccagagatgcagaaacacttcctgctgttctccctcgtgaatcacctgagcaaccaggacgattacattgagaaggcccaccagccatacgatatcggcgagggcctgttctttcatcgcatcgccagtaccttcctgaacatcagcggaatcctgaggaatatgaagttttacacctatcagagcaaacggctggtggagcagagaggcgagctgaagagagagaaggatattttcgcctgggaggagccatttcagggcaactcctacttcgagatcaatgggcacaaaggagtgattggcgaggacgagctgaaagagctctgctatgccttcctgatcggcaatcaggacgccaacaaggtggagggcaggattacacagtttctggagaagttcagaaacgccaatagcgtgcagcaggtgaaggacgatgagatgctgaaacccgaatacttccccgccaattacttcgcagagagcggggtgggacggatcaaggatcgagtgctgaataggctgaacaaggctatcaagtccaacaaggccaaaaaaggggagattatcgcctatgacaagatgagggaggtgatggccttcatcaacaactcactgcctgtggacgagaagctgaagcccaaagattacaagaggtatctgggcatggtccggttctgggacagggagaaagataatatcaagagggagtttgagactaaggagtggagcaagtatctgccctccaacttctggaccgccaagaacctggagcgcgtgtatggcctggcacgcgagaaaaacgccgaactgttcaataagctgaaagccgatgtggagaagatggacgagcgggagctggagaagtaccagaagatcaatgatgcaaaggacctggccaatttgagacggctcgcctccgactttggagtgaagtgggaggagaaagactgggatgaatacagcggacagatcaagaagcagattacagactcccagaaactgactatcatgaaacagaggatcaccgctggcctgaaaaaaaaacacggaatcgagaatctgaatctgaggatcaccatcgacattaacaaatcccggaaggcggtgctgaacagaattgccatcccccgggggttcgtgaagaggcacatcctgggttggcaggagagcgagaaagtgagcaagaagattagggaggctgaatgtgaaattctgctgtccaaggagtacgaggagctgagcaaacagtttttccagtctaaagactacgacaagatgaccaggataaacggactgtacgagaagaacaagctgatcgccctgatggccgtgtacctcatggggcagctgagaatcctgttcaaagagcacactaagctggacgacatcaccaagactaccgtggacttcaagatcagtgataaggtcaccgtgaaaatccccttcagcaattaccctagcctggtgtacacaatgtcctccaaatacgtggacaatatcgggaactacggattctccaacaaggataaggataagcccattctgggcaaaatcgatgtgatcgaaaaacagagaatggagttcatcaaggaagtgctggggtttgagaagtatcttttcgacgacaagattatcgataaaagtaagttcgcagacaccgccactcacatctcttttgccgagatcgtggaggagctggtggagaagggctgggacaaggaccgcctgactaagctcaaagacgctagaaacaaagccctgcacggcgagatcctgacgggcaccagctttgacgaaaccaagtctctgatcaatgagttgaagaagacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggttggagaactggatgtagatgggctggctgtgatagacctcgatttgtggggtagtaacagcttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 95 - DNA, 70 bp, synthetic construct
  source 1..70: mol_type = other DNA
tgccgttcttctgcttgtcggccatgatatagacgttgtggctgttgtagttgtactccagcttgtgccc

SEQ ID NO: 96 - DNA, 6793 bp, synthetic construct
  source 1..6793: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgccggcagctaagaaaaagaaactggatggcagcgtcgacatgagcaaggacaagaaaaccaaggccaagagaatgggcgtgaaggccctgctggcccacggcgaggacaagctgaccatgaccaccttcggcaagggcaacagaagcaagatcgagttcaccgagggctaccacggcagagccctggagacacccaagcacttcggcatcagaggcttcgaggtgagaagaatcgacgagaacgtggacctgtgcggcgacctggaggagggcaagaccatcgaggccctgctggtgaaccccagcgagaaggtgggcgaggactacctgaagctgaagggcaccctggagaagagattcttcggcagagagttcccccacgacaacatcagaatccagctgatctacaacatcctggacatctacaagatcctgggcatgaacgtggccgacatcctgtacgccctgggcaacatgcaggacaccgagctggacatcgacatgttcggccagagcctgaacaacgaggacaacctgaaggagtgcctgaagagaatgaggccctacatgggctacttcggcgacatcttcaagatcagccccaagggcgagaacatcgccgacagagagcacaacaagaaggtgctgagatgcatcagcgtgctggcaaacgccaccgccgccgacaagcaggacgagtacccctggttcaagagcagcgacatctacgagacaaagatcttcaaggccgacatgtggaagatcatcaaggaccagtacagagagaagatcaagaaggtgaacaaggacttcctgagcaagaacgccgtgaacatggccatcctgttcgacctgctgaacgccagagacgtggagcagaagaagcagatcaccgacgagttctacagattcaccatcagaaaggacggcaagaacctgggcatgaacctggtgaagatcagagagatcatcatcgacagatacgccagcggcctgagagacaagaagcacgacccccacagacagaagatcaacgtgatcgccgacttcctgatcttcagagccctgagccagaaccagggcatcatcgacaagaccgtgagcagcctgagactgaccaaggacgaggaggagaaggaccacgtgtaccagaacgccgccgagctggtgtggggcatggtgagcaactgcctgaccccctacttcaacgaccccaagaacaagtacatcctgaagtacaaggacgccaagacccccggcgacttcgaggactggatcaccagcaagatcagcgaggacgacggcgagcccttcgtgaaggtgctgagcttcctgtgcaacttcctggagggcaaggagatcaacgagctgctgaccgcctacatccacaagttcgagtgcatccaggacttcctgaacgtgatcagcagcctgggcgagaacgtgcagttccagcccagattcgccctgttcaacaacgccagcttcgcccagaacgtggccgtgcagctgagaatcctggccagcatcggcaagatgaagcccgacctgaccgaggccaagaggcccctgtacaaggccgccatcagaatgctgtgcccccccgagaagtgggagaagtacaccagcgacgagtggctggagaagaacatgctgctgaacagcgaggacagaaagaacgacaagaagaagaagcaggtgaaccccttcagaaacttcatcgccggcaacgtgatcgagagcagaagattcatgtacctggtgagatacagcaagcccaaggccgtgagagccatcatgcagaacagaagcatcgtgaactacgtgctgcacagactgcccagcgagcaggtgcacagatacgccagcgtgttccccgagaacttcgccgacctggagcaggagatcgacttcctgaccaagaagctgttcgagttcagcttcgaggagctgctgcacgagaaggacgtgatcctgaacaacagcagaagccacaagcccagcctggagatcgagagactgaaggccatcaccggcctgtacctgagcgtggcctacatcgccatcaagaacatcgtgaaggccaacgccagatactacatcgccttcgccgtgttcgagagagacaaggagctggtgaaggccaaggacgccagaatccagaccaagatccccgagacagacttccccgactacttctgcctgacccagtactacctggacagagacgaggagaagaagttccccggcgaccccagagacaaggaggccttcttcgagcacctggcaaagaccaagagagccttcagcaagcagtggagagagtggctgaacgagaagatcgccgacgccaagagcagccaggccaccggcctgctgctgagagaggccgcaaacgacgtggaggccctgaacgtgctgagagccatccccgactacatccaggacttcagacacggcgagaagggcgagacagccatgaacagctacttcgagctgtaccactacctgatgcagagactgatgctgaagaacaccgagctggacctgagccactggagcggctggatcatgagaagcggcagacccgacagagacttgatccagatcgccttcgtgagcctggcctacaacctgcccagatacagaaacctgaccaaggagcaccacttcgacgacaccgtgctgcagaagatcagagagaaggagagcctggacacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagataactctacaaacctgtagggttctgagacggagaccacggcaggtctcattttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 97 - DNA, 7830 bp, synthetic construct
  source 1..7830: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgagcaaggacaagaaaaccaaggccaagagaatgggcgtgaaggccctgctggcccacggcgaggacaagctgaccatgaccaccttcggcaagggcaacagaagcaagatcgagttcaccgagggctaccacggcagagccctggagacacccaagcacttcggcatcagaggcttcgaggtgagaagaatcgacgagaacgtggacctgtgcggcgacctggaggagggcaagaccatcgaggccctgctggtgaaccccagcgagaaggtgggcgaggactacctgaagctgaagggcaccctggagaagagattcttcggcagagagttcccccacgacaacatcagaatccagctgatctacaacatcctggacatctacaagatcctgggcatgaacgtggccgacatcctgtacgccctgggcaacatgcaggacaccgagctggacatcgacatgttcggccagagcctgaacaacgaggacaacctgaaggagtgcctgaagagaatgaggccctacatgggctacttcggcgacatcttcaagatcagccccaagggcgagaacatcgccgacagagagcacaacaagaaggtgctgagatgcatcagcgtgctggcaaacgccaccgccgccgacaagcaggacgagtacccctggttcaagagcagcgacatctacgagacaaagatcttcaaggccgacatgtggaagatcatcaaggaccagtacagagagaagatcaagaaggtgaacaaggacttcctgagcaagaacgccgtgaacatggccatcctgttcgacctgctgaacgccagagacgtggagcagaagaagcagatcaccgacgagttctacagattcaccatcagaaaggacggcaagaacctgggcatgaacctggtgaagatcagagagatcatcatcgacagatacgccagcggcctgagagacaagaagcacgacccccacagacagaagatcaacgtgatcgccgacttcctgatcttcagagccctgagccagaaccagggcatcatcgacaagaccgtgagcagcctgagactgaccaaggacgaggaggagaaggaccacgtgtaccagaacgccgccgagctggtgtggggcatggtgagcaactgcctgaccccctacttcaacgaccccaagaacaagtacatcctgaagtacaaggacgccaagacccccggcgacttcgaggactggatcaccagcaagatcagcgaggacgacggcgagcccttcgtgaaggtgctgagcttcctgtgcaacttcctggagggcaaggagatcaacgagctgctgaccgcctacatccacaagttcgagtgcatccaggacttcctgaacgtgatcagcagcctgggcgagaacgtgcagttccagcccagattcgccctgttcaacaacgccagcttcgcccagaacgtggccgtgcagctgagaatcctggccagcatcggcaagatgaagcccgacctgaccgaggccaagaggcccctgtacaaggccgccatcagaatgctgtgcccccccgagaagtgggagaagtacaccagcgacgagtggctggagaagaacatgctgctgaacagcgaggacagaaagaacgacaagaagaagaagcaggtgaaccccttcagaaacttcatcgccggcaacgtgatcgagagcagaagattcatgtacctggtgagatacagcaagcccaaggccgtgagagccatcatgcagaacagaagcatcgtgaactacgtgctgcacagactgcccagcgagcaggtgcacagatacgccagcgtgttccccgagaacttcgccgacctggagcaggagatcgacttcctgaccaagaagctgttcgagttcagcttcgaggagctgctgcacgagaaggacgtgatcctgaacaacagcagaagccacaagcccagcctggagatcgagagactgaaggccatcaccggcctgtacctgagcgtggcctacatcgccatcaagaacatcgtgaaggccaacgccagatactacatcgccttcgccgtgttcgagagagacaaggagctggtgaaggccaaggacgccagaatccagaccaagatccccgagacagacttccccgactacttctgcctgacccagtactacctggacagagacgaggagaagaagttccccggcgaccccagagacaaggaggccttcttcgagcacctggcaaagaccaagagagccttcagcaagcagtggagagagtggctgaacgagaagatcgccgacgccaagagcagccaggccaccggcctgctgctgagagaggccgcaaacgacgtggaggccctgaacgtgctgagagccatccccgactacatccaggacttcagacacggcgagaagggcgagacagccatgaacagctacttcgagctgtaccactacctgatgcagagactgatgctgaagaacaccgagctggacctgagccactggagcggctggatcatgagaagcggcagacccgacagagacttgatccagatcgccttcgtgagcctggcctacaacctgcccagatacagaaacctgaccaaggagcaccacttcgacgacaccgtgctgcagaagatcagagagaaggagagcctggacctgcctccacttgaaagactgacactgggatcccagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaggcaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgtaagaattccccttgagcatctgacttctggctaataaaggaaatttattttcattgcaatagtgtgttggaattttttgtgtctctcaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagataactctacaaacctgtagggttctgagactgccgttcttctgcttgtcggccatgatatagacgttgtggctgttgtagttgtactccagcttgtgcccttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 98 - DNA, 7892 bp, synthetic construct
  source 1..7892: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgagcaaggacaagaaaaccaaggccaagagaatgggcgtgaaggccctgctggcccacggcgaggacaagctgaccatgaccaccttcggcaagggcaacagaagcaagatcgagttcaccgagggctaccacggcagagccctggagacacccaagcacttcggcatcagaggcttcgaggtgagaagaatcgacgagaacgtggacctgtgcggcgacctggaggagggcaagaccatcgaggccctgctggtgaaccccagcgagaaggtgggcgaggactacctgaagctgaagggcaccctggagaagagattcttcggcagagagttcccccacgacaacatcagaatccagctgatctacaacatcctggacatctacaagatcctgggcatgaacgtggccgacatcctgtacgccctgggcaacatgcaggacaccgagctggacatcgacatgttcggccagagcctgaacaacgaggacaacctgaaggagtgcctgaagagaatgaggccctacatgggctacttcggcgacatcttcaagatcagccccaagggcgagaacatcgccgacagagagcacaacaagaaggtgctgagatgcatcagcgtgctggcaaacgccaccgccgccgacaagcaggacgagtacccctggttcaagagcagcgacatctacgagacaaagatcttcaaggccgacatgtggaagatcatcaaggaccagtacagagagaagatcaagaaggtgaacaaggacttcctgagcaagaacgccgtgaacatggccatcctgttcgacctgctgaacgccagagacgtggagcagaagaagcagatcaccgacgagttctacagattcaccatcagaaaggacggcaagaacctgggcatgaacctggtgaagatcagagagatcatcatcgacagatacgccagcggcctgagagacaagaagcacgacccccacagacagaagatcaacgtgatcgccgacttcctgatcttcagagccctgagccagaaccagggcatcatcgacaagaccgtgagcagcctgagactgaccaaggacgaggaggagaaggaccacgtgtaccagaacgccgccgagctggtgtggggcatggtgagcaactgcctgaccccctacttcaacgaccccaagaacaagtacatcctgaagtacaaggacgccaagacccccggcgacttcgaggactggatcaccagcaagatcagcgaggacgacggcgagcccttcgtgaaggtgctgagcttcctgtgcaacttcctggagggcaaggagatcaacgagctgctgaccgcctacatccacaagttcgagtgcatccaggacttcctgaacgtgatcagcagcctgggcgagaacgtgcagttccagcccagattcgccctgttcaacaacgccagcttcgcccagaacgtggccgtgcagctgagaatcctggccagcatcggcaagatgaagcccgacctgaccgaggccaagaggcccctgtacaaggccgccatcagaatgctgtgcccccccgagaagtgggagaagtacaccagcgacgagtggctggagaagaacatgctgctgaacagcgaggacagaaagaacgacaagaagaagaagcaggtgaaccccttcagaaacttcatcgccggcaacgtgatcgagagcagaagattcatgtacctggtgagatacagcaagcccaaggccgtgagagccatcatgcagaacagaagcatcgtgaactacgtgctgcacagactgcccagcgagcaggtgcacagatacgccagcgtgttccccgagaacttcgccgacctggagcaggagatcgacttcctgaccaagaagctgttcgagttcagcttcgaggagctgctgcacgagaaggacgtgatcctgaacaacagcagaagccacaagcccagcctggagatcgagagactgaaggccatcaccggcctgtacctgagcgtggcctacatcgccatcaagaacatcgtgaaggccaacgccagatactacatcgccttcgccgtgttcgagagagacaaggagctggtgaaggccaaggacgccagaatccagaccaagatccccgagacagacttccccgactacttctgcctgacccagtactacctggacagagacgaggagaagaagttccccggcgaccccagagacaaggaggccttcttcgagcacctggcaaagaccaagagagccttcagcaagcagtggagagagtggctgaacgagaagatcgccgacgccaagagcagccaggccaccggcctgctgctgagagaggccgcaaacgacgtggaggccctgaacgtgctgagagccatccccgactacatccaggacttcagacacggcgagaagggcgagacagccatgaacagctacttcgagctgtaccactacctgatgcagagactgatgctgaagaacaccgagctggacctgagccactggagcggctggatcatgagaagcggcagacccgacagagacttgatccagatcgccttcgtgagcctggcctacaacctgcccagatacagaaacctgaccaaggagcaccacttcgacgacaccgtgctgcagaagatcagagagaaggagagcctggacggatcccttcaactgcctccacttgaaagactgacactgggatcccagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaggcaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgtaagcggccgctcgagtctagagggcccgtttaaacccgctgatcagcctcgagaattccccttgagcatctgacttctggctaataaaggaaatttattttcattgcaatagtgtgttggaattttttgtgtctctcaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagataactctacaaacctgtagggttctgagactgccgttcttctgcttgtcggccatgatatagacgttgtggctgttgtagttgtactccagcttgtgcccttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 99 - DNA, 7943 bp, synthetic construct
  source 1..7943: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgagcaaggacaagaaaaccaaggccaagagaatgggcgtgaaggccctgctggcccacggcgaggacaagctgaccatgaccaccttcggcaagggcaacagaagcaagatcgagttcaccgagggctaccacggcagagccctggagacacccaagcacttcggcatcagaggcttcgaggtgagaagaatcgacgagaacgtggacctgtgcggcgacctggaggagggcaagaccatcgaggccctgctggtgaaccccagcgagaaggtgggcgaggactacctgaagctgaagggcaccctggagaagagattcttcggcagagagttcccccacgacaacatcagaatccagctgatctacaacatcctggacatctacaagatcctgggcatgaacgtggccgacatcctgtacgccctgggcaacatgcaggacaccgagctggacatcgacatgttcggccagagcctgaacaacgaggacaacctgaaggagtgcctgaagagaatgaggccctacatgggctacttcggcgacatcttcaagatcagccccaagggcgagaacatcgccgacagagagcacaacaagaaggtgctgagatgcatcagcgtgctggcaaacgccaccgccgccgacaagcaggacgagtacccctggttcaagagcagcgacatctacgagacaaagatcttcaaggccgacatgtggaagatcatcaaggaccagtacagagagaagatcaagaaggtgaacaaggacttcctgagcaagaacgccgtgaacatggccatcctgttcgacctgctgaacgccagagacgtggagcagaagaagcagatcaccgacgagttctacagattcaccatcagaaaggacggcaagaacctgggcatgaacctggtgaagatcagagagatcatcatcgacagatacgccagcggcctgagagacaagaagcacgacccccacagacagaagatcaacgtgatcgccgacttcctgatcttcagagccctgagccagaaccagggcatcatcgacaagaccgtgagcagcctgagactgaccaaggacgaggaggagaaggaccacgtgtaccagaacgccgccgagctggtgtggggcatggtgagcaactgcctgaccccctacttcaacgaccccaagaacaagtacatcctgaagtacaaggacgccaagacccccggcgacttcgaggactggatcaccagcaagatcagcgaggacgacggcgagcccttcgtgaaggtgctgagcttcctgtgcaacttcctggagggcaaggagatcaacgagctgctgaccgcctacatccacaagttcgagtgcatccaggacttcctgaacgtgatcagcagcctgggcgagaacgtgcagttccagcccagattcgccctgttcaacaacgccagcttcgcccagaacgtggccgtgcagctgagaatcctggccagcatcggcaagatgaagcccgacctgaccgaggccaagaggcccctgtacaaggccgccatcagaatgctgtgcccccccgagaagtgggagaagtacaccagcgacgagtggctggagaagaacatgctgctgaacagcgaggacagaaagaacgacaagaagaagaagcaggtgaaccccttcagaaacttcatcgccggcaacgtgatcgagagcagaagattcatgtacctggtgagatacagcaagcccaaggccgtgagagccatcatgcagaacagaagcatcgtgaactacgtgctgcacagactgcccagcgagcaggtgcacagatacgccagcgtgttccccgagaacttcgccgacctggagcaggagatcgacttcctgaccaagaagctgttcgagttcagcttcgaggagctgctgcacgagaaggacgtgatcctgaacaacagcagaagccacaagcccagcctggagatcgagagactgaaggccatcaccggcctgtacctgagcgtggcctacatcgccatcaagaacatcgtgaaggccaacgccagatactacatcgccttcgccgtgttcgagagagacaaggagctggtgaaggccaaggacgccagaatccagaccaagatccccgagacagacttccccgactacttctgcctgacccagtactacctggacagagacgaggagaagaagttccccggcgaccccagagacaaggaggccttcttcgagcacctggcaaagaccaagagagccttcagcaagcagtggagagagtggctgaacgagaagatcgccgacgccaagagcagccaggccaccggcctgctgctgagagaggccgcaaacgacgtggaggccctgaacgtgctgagagccatccccgactacatccaggacttcagacacggcgagaagggcgagacagccatgaacagctacttcgagctgtaccactacctgatgcagagactgatgctgaagaacaccgagctggacctgagccactggagcggctggatcatgagaagcggcagacccgacagagacttgatccagatcgccttcgtgagcctggcctacaacctgcccagatacagaaacctgaccaaggagcaccacttcgacgacaccgtgctgcagaagatcagagagaaggagagcctggacgccgaggccgccgccaaggaggccgccgccaaggaggccgccgccaaggccggatcccttcaactgcctccacttgaaagactgacactgggatcccagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaggcaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgtaagcggccgctcgagtctagagggcccgtttaaacccgctgatcagcctcgagaattccccttgagcatctgacttctggctaataaaggaaatttattttcattgcaatagtgtgttggaattttttgtgtctctcaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagataactctacaaacctgtagggttctgagactgccgttcttctgcttgtcggccatgatatagacgttgtggctgttgtagttgtactccagcttgtgcccttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 100 - DNA, 7937 bp, synthetic construct
  source 1..7937: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgagcaaggacaagaaaaccaaggccaagagaatgggcgtgaaggccctgctggcccacggcgaggacaagctgaccatgaccaccttcggcaagggcaacagaagcaagatcgagttcaccgagggctaccacggcagagccctggagacacccaagcacttcggcatcagaggcttcgaggtgagaagaatcgacgagaacgtggacctgtgcggcgacctggaggagggcaagaccatcgaggccctgctggtgaaccccagcgagaaggtgggcgaggactacctgaagctgaagggcaccctggagaagagattcttcggcagagagttcccccacgacaacatcagaatccagctgatctacaacatcctggacatctacaagatcctgggcatgaacgtggccgacatcctgtacgccctgggcaacatgcaggacaccgagctggacatcgacatgttcggccagagcctgaacaacgaggacaacctgaaggagtgcctgaagagaatgaggccctacatgggctacttcggcgacatcttcaagatcagccccaagggcgagaacatcgccgacagagagcacaacaagaaggtgctgagatgcatcagcgtgctggcaaacgccaccgccgccgacaagcaggacgagtacccctggttcaagagcagcgacatctacgagacaaagatcttcaaggccgacatgtggaagatcatcaaggaccagtacagagagaagatcaagaaggtgaacaaggacttcctgagcaagaacgccgtgaacatggccatcctgttcgacctgctgaacgccagagacgtggagcagaagaagcagatcaccgacgagttctacagattcaccatcagaaaggacggcaagaacctgggcatgaacctggtgaagatcagagagatcatcatcgacagatacgccagcggcctgagagacaagaagcacgacccccacagacagaagatcaacgtgatcgccgacttcctgatcttcagagccctgagccagaaccagggcatcatcgacaagaccgtgagcagcctgagactgaccaaggacgaggaggagaaggaccacgtgtaccagaacgccgccgagctggtgtggggcatggtgagcaactgcctgaccccctacttcaacgaccccaagaacaagtacatcctgaagtacaaggacgccaagacccccggcgacttcgaggactggatcaccagcaagatcagcgaggacgacggcgagcccttcgtgaaggtgctgagcttcctgtgcaacttcctggagggcaaggagatcaacgagctgctgaccgcctacatccacaagttcgagtgcatccaggacttcctgaacgtgatcagcagcctgggcgagaacgtgcagttccagcccagattcgccctgttcaacaacgccagcttcgcccagaacgtggccgtgcagctgagaatcctggccagcatcggcaagatgaagcccgacctgaccgaggccaagaggcccctgtacaaggccgccatcagaatgctgtgcccccccgagaagtgggagaagtacaccagcgacgagtggctggagaagaacatgctgctgaacagcgaggacagaaagaacgacaagaagaagaagcaggtgaaccccttcagaaacttcatcgccggcaacgtgatcgagagcagaagattcatgtacctggtgagatacagcaagcccaaggccgtgagagccatcatgcagaacagaagcatcgtgaactacgtgctgcacagactgcccagcgagcaggtgcacagatacgccagcgtgttccccgagaacttcgccgacctggagcaggagatcgacttcctgaccaagaagctgttcgagttcagcttcgaggagctgctgcacgagaaggacgtgatcctgaacaacagcagaagccacaagcccagcctggagatcgagagactgaaggccatcaccggcctgtacctgagcgtggcctacatcgccatcaagaacatcgtgaaggccaacgccagatactacatcgccttcgccgtgttcgagagagacaaggagctggtgaaggccaaggacgccagaatccagaccaagatccccgagacagacttccccgactacttctgcctgacccagtactacctggacagagacgaggagaagaagttccccggcgaccccagagacaaggaggccttcttcgagcacctggcaaagaccaagagagccttcagcaagcagtggagagagtggctgaacgagaagatcgccgacgccaagagcagccaggccaccggcctgctgctgagagaggccgcaaacgacgtggaggccctgaacgtgctgagagccatccccgactacatccaggacttcagacacggcgagaagggcgagacagccatgaacagctacttcgagctgtaccactacctgatgcagagactgatgctgaagaacaccgagctggacctgagccactggagcggctggatcatgagaagcggcagacccgacagagacttgatccagatcgccttcgtgagcctggcctacaacctgcccagatacagaaacctgaccaaggagcaccacttcgacgacaccgtgctgcagaagatcagagagaaggagagcctggacggtggcggaggttctggtggcggaggttctggtggcggaggttctggatcccttcaactgcctccacttgaaagactgacactgggatcccagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaggcaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgtaagcggccgctcgagtctagagggcccgtttaaacccgctgatcagcctcgagaattccccttgagcatctgacttctggctaataaaggaaatttattttcattgcaatagtgtgttggaattttttgtgtctctcaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagataactctacaaacctgtagggttctgagactgccgttcttctgcttgtcggccatgatatagacgttgtggctgttgtagttgtactccagcttgtgcccttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 101 - DNA, 4828 bp, synthetic construct; identical to SEQ ID NO: 13
  source 1..4828: mol_type = other DNA

SEQ ID NO: 102 - DNA, 7141 bp, synthetic construct
  source 1..7141: mol_type = other DNA
ttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacctgccgttcttctgcttgtcggccatgatatagacgttgtggctgttgtagttgtactccagcttgtgcccgttgtggaaggtccagttttgaggggctattacaacttttttgaattctcgacctcgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggccgcggctcgaggggg

SEQ ID NO: 103 - DNA, 16 bp, synthetic construct
  source 1..16: mol_type = other DNA
agggcgaggagctgtt

SEQ ID NO: 104 - DNA, 19 bp, synthetic construct
  source 1..19: mol_type = other DNA
gtacagctcgtccatgccg

SEQ ID NO: 105 - DNA, 33 bp, synthetic construct
  source 1..33: mol_type = other DNA
aaccaaggccaaggcaatgggcgtgaaggccct

SEQ ID NO: 106 - DNA, 33 bp, synthetic construct
  source 1..33: mol_type = other DNA
attgccttggccttggttttcttgtccttgctc

SEQ ID NO: 107 - DNA, 33 bp, synthetic construct
  source 1..33: mol_type = other DNA
cgccagaagcaagatcgagttcaccgagggcta

SEQ ID NO: 108 - DNA, 35 bp, synthetic construct
  source 1..35: mol_type = other DNA
tcgatcttgcttctggcgcccttgccgaaggtggt

SEQ ID NO: 109 - DNA, 33 bp, synthetic construct
  source 1..33: mol_type = other DNA
caacgcaagcaagatcgagttcaccgagggcta

SEQ ID NO: 110 - DNA, 33 bp, synthetic construct
  source 1..33: mol_type = other DNA
tcgatcttgcttgcgttgcccttgccgaaggtg

SEQ ID NO: 111 - DNA, 33 bp, synthetic construct
  source 1..33: mol_type = other DNA
taccacggcgcagccctggagacacccaagcac

SEQ ID NO: 112 - DNA, 33 bp, synthetic construct
  source 1..33: mol_type = other DNA
cagggctgcgccgtggtagccctcggtgaactc

SEQ ID NO: 113 - DNA, 40 bp, synthetic construct
  source 1..40: mol_type = other DNA
aagcacttcggcatcgcaggcttcgaggtgagaagaatcg

SEQ ID NO: 114 - DNA, 33 bp, synthetic construct
  source 1..33: mol_type = other DNA
tgcgatgccgaagtgcttgggtgtctccagggc

SEQ ID NO: 115 - DNA, 34 bp, synthetic construct
  source 1..34: mol_type = other DNA
ttcgaggtgagcagaatcgacgagaacgtggacc

SEQ ID NO: 116 - DNA, 33 bp, synthetic construct
  source 1..33: mol_type = other DNA
gattctgctcacctcgaagcctctgatgccgaa

SEQ ID NO: 117 - DNA, 33 bp, synthetic construct
  source 1..33: mol_type = other DNA
aggtgagaagcaatcgacgagaacgtggacctg

SEQ ID NO: 118 - DNA, 33 bp, synthetic construct
  source 1..33: mol_type = other DNA
gtcgattgcttctcacctcgaagcctctgatgc

SEQ ID NO: 119 - DNA, 33 bp, synthetic construct
  source 1..33: mol_type = other DNA
aagaatcgacgaggccgtggacctgtgcggcga

SEQ ID NO: 120 - DNA, 33 bp, synthetic construct
  source 1..33: mol_type = other DNA
acggcctcgtcgattcttctcacctcgaagcct

SEQ ID NO: 121 - DNA, 33 bp, synthetic construct
  source 1..33: mol_type = other DNA
tggtggcccccagcgagaaggtgggcgaggact

SEQ ID NO: 122 - DNA, 33 bp, synthetic construct
  source 1..33: mol_type = other DNA
tctcgctgggggccaccagcagggcctcgatgg

SEQ ID NO: 123 - DNA, 34 bp, synthetic construct
  source 1..34: mol_type = other DNA
aagaacgccgtggccatggccatcctgttcgacc

SEQ ID NO: 124 - DNA, 33 bp, synthetic construct
  source 1..33: mol_type = other DNA
catggccacggcgttcttgctcaggaagtcctt

SEQ ID NO: 125 - DNA, 34 bp, synthetic construct
  source 1..34: mol_type = other DNA
tgttcgacctgctggccgccagagacgtggagca

SEQ ID NO: 126 - DNA, 33 bp, synthetic construct
  source 1..33: mol_type = other DNA
cggccagcaggtcgaacaggatggccatgttca

SEQ ID NO: 127 - DNA, 33 bp, synthetic construct
  source 1..33: mol_type = other DNA
cgcagacgtggagcagaagaagcagatcaccga

SEQ ID NO: 128 - DNA, 36 bp, synthetic construct
  source 1..36: mol_type = other DNA
ttctgctccacgtctgcggcgttcagcaggtcgaac

SEQ ID NO: 129 - DNA, 33 bp, synthetic construct
  source 1..33: mol_type = other DNA
gagttctacgcattcaccatcagaaaggacggc

SEQ ID NO: 130 - DNA, 33 bp, synthetic construct
  source 1..33: mol_type = other DNA
ggtgaatgcgtagaactcgtcggtgatctgctt

SEQ ID NO: 131 - DNA, 33 bp, synthetic construct
  source 1..33: mol_type = other DNA
ttcaccatcgcaaaggacggcaagaacctgggc

SEQ ID NO: 132 - DNA, 34 bp, synthetic construct
  source 1..34: mol_type = other DNA
gtcctttgcgatggtgaatctgtagaactcgtcg

SEQ ID NO: 133 - DNA, 33 bp, synthetic construct
  source 1..33: mol_type = other DNA
caaggccctgggcatgaacctggtgaagatcag

SEQ ID NO: 134 - DNA, 34 bp, synthetic construct
  source 1..34: mol_type = other DNA
ttcatgcccagggccttgccgtcctttctgatgg

SEQ ID NO: 135 - DNA, 33 bp, synthetic construct
  source 1..33: mol_type = other DNA
catggccctggtgaagatcagagagatcatcat

SEQ ID NO: 136 - DNA, 33 bp, synthetic construct
  source 1..33: mol_type = other DNA
atcttcaccagggccatgcccaggttcttgccg

SEQ ID NO: 137 - DNA, 35 bp, synthetic construct
  source 1..35: mol_type = other DNA
gtgaagatcgcagagatcatcatcgacagatacgc

SEQ ID NO: 138 - DNA, 33 bp, synthetic construct
  source 1..33: mol_type = other DNA
gatctctgcgatcttcaccaggttcatgcccag

SEQ ID NO: 139 - DNA, 33 bp, synthetic construct
  source 1..33: mol_type = other DNA
tcatcatcgacgcatacgccagcggcctgagag

SEQ ID NO: 140 - DNA, 33 bp, synthetic construct
  source 1..33: mol_type = other DNA
cgtatgcgtcgatgatgatctctctgatcttca

SEQ ID NO: 141 - DNA, 33 bp, synthetic construct
  source 1..33: mol_type = other DNA
cctggcagacaagaagcacgacccccacagaca

SEQ ID NO: 142 - DNA, 33 bp, synthetic construct
  source 1..33: mol_type = other DNA
tgcttcttgtctgccaggccgctggcgtatctg

SEQ ID NO: 143 - DNA, 33 bp, synthetic construct
  source 1..33: mol_type = other DNA
ccacgcacagaagatcaacgtgatcgccgactt

SEQ ID NO: 144 - DNA, 33 bp, synthetic construct
  source 1..33: mol_type = other DNA
ttgatcttctgtgcgtgggggtcgtgcttcttg

SEQ ID NO: 145 - DNA, 33 bp, synthetic construct
  source 1..33: mol_type = other DNA
acagaagatcgccgtgatcgccgacttcctgat

SEQ ID NO: 146 - DNA, 33 bp, synthetic construct
  source 1..33: mol_type = other DNA
atcacggcgatcttctgtctgtgggggtcgtgc

SEQ ID NO: 147 - DNA, 36 bp, synthetic construct
  source 1..36: mol_type = other DNA
acttcctgatcttcgcagccctgagccagaaccagg

SEQ ID NO: 148 - DNA, 32 bp, synthetic construct
  source 1..32: mol_type = other DNA
ctgcgaagatcaggaagtcggcgatcacgttg

SEQ ID NO: 149 - DNA, 33 bp, synthetic construct
  source 1..33: mol_type = other DNA
ccaggcccagggcatcatcgacaagaccgtgag

SEQ ID NO: 150 - DNA, 34 bp, synthetic construct
  source 1..34: mol_type = other DNA
atgatgccctgggcctggctcagggctctgaaga

SEQ ID NO: 151 - DNA, 33 bp, synthetic construct
  source 1..33: mol_type = other DNA
cagcctggcactgaccaaggacgaggaggagaa

SEQ ID NO: 152 - DNA, 33 bp, synthetic construct
  source 1..33: mol_type = other DNA
ttggtcagtgccaggctgctcacggtcttgtcg

SEQ ID NO: 153 - DNA, 34 bp, synthetic construct
  source 1..34: mol_type = other DNA
accacgtgtaccaggccgccgccgagctggtgtg

SEQ ID NO: 154 - DNA, 33 bp, synthetic construct
  source 1..33: mol_type = other DNA
cggcctggtacacgtggtccttctcctcctcgt

SEQ ID NO: 155 - DNA, 33 bp, synthetic construct
  source 1..33: mol_type = other DNA
tggtgagcgcctgcctgaccccctacttcaacg

SEQ ID NO: 156 - DNA, 33 bp, synthetic construct
  source 1..33: mol_type = other DNA
tcaggcaggcgctcaccatgccccacaccagct

SEQ ID NO: 157 - DNA, 33 bp, synthetic construct
  source 1..33: mol_type = other DNA
ctacttcgccgaccccaagaacaagtacatcct

SEQ ID NO: 158 - DNA, 33 bp, synthetic construct
  source 1..33: mol_type = other DNA
ttggggtcggcgaagtagggggtcaggcagttg

SEQ ID NO: 159 - DNA, 33 bp, synthetic construct
  source 1..33: mol_type = other DNA
caaggccaagtacatcctgaagtacaaggacgc

SEQ ID NO: 160 - DNA, 35 bp, synthetic construct
  source 1..35: mol_type = other DNA
aggatgtacttggccttggggtcgttgaagtaggg

SEQ ID NO: 161 - DNA, 6798 bp, synthetic construct
  source 1..6798: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgccggcagctaagaaaaagaaactggatggcagcgtcgacatgagcaaggacaagaaaaccaaggccaagagaatgggcgtgaaggccctgctggcccacggcgaggacaagctgaccatgaccaccttcggcaagggcaacagaagcaagatcgagttcaccgagggctaccacggcagagccctggagacacccaagcacttcggcatcagaggcttcgaggtgagaagaatcgacgagaacgtggacctgtgcggcgacctggaggagggcaagaccatcgaggccctgctggtgaaccccagcgagaaggtgggcgaggactacctgaagctgaagggcaccctggagaagagattcttcggcagagagttcccccacgacaacatcagaatccagctgatctacaacatcctggacatctacaagatcctgggcatgaacgtggccgacatcctgtacgccctgggcaacatgcaggacaccgagctggacatcgacatgttcggccagagcctgaacaacgaggacaacctgaaggagtgcctgaagagaatgaggccctacatgggctacttcggcgacatcttcaagatcagccccaagggcgagaacatcgccgacagagagcacaacaagaaggtgctgagatgcatcagcgtgctgagaaacgccaccgcccacgacaagcaggacgagtacccctggttcaagagcagcgacatctacgagacaaagatcttcaaggccgacatgtggaagatcatcaaggaccagtacagagagaagatcaagaaggtgaacaaggacttcctgagcaagaacgccgtgaacatggccatcctgttcgacctgctgaacgccagagacgtggagcagaagaagcagatcaccgacgagttctacagattcaccatcagaaaggacggcaagaacctgggcatgaacctggtgaagatcagagagatcatcatcgacagatacgccagcggcctgagagacaagaagcacgacccccacagacagaagatcaacgtgatcgccgacttcctgatcttcagagccctgagccagaaccagggcatcatcgacaagaccgtgagcagcctgagactgaccaaggacgaggaggagaaggaccacgtgtaccagaacgccgccgagctggtgtggggcatggtgagcaactgcctgaccccctacttcaacgaccccaagaacaagtacatcctgaagtacaaggacgccaagacccccggcgacttcgaggactggatcaccagcaagatcagcgaggacgacggcgagcccttcgtgaaggtgctgagcttcctgtgcaacttcctggagggcaaggagatcaacgagctgctgaccgcctacatccacaagttcgagtgcatccaggacttcctgaacgtgatcagcagcctgggcgagaacgtgcagttccagcccagattcgccctgttcaacaacgccagcttcgcccagaacgtggccgtgcagctgagaatcctggccagcatcggcaagatgaagcccgacctgaccgaggccaagaggcccctgtacaaggccgccatcagaatgctgtgcccccccgagaagtgggagaagtacaccagcgacgagtggctggagaagaacatgctgctgaacagcgaggacagaaagaacgacaagaagaagaagcaggtgaaccccttcagaaacttcatcgccggcaacgtgatcgagagcagaagattcatgtacctggtgagatacagcaagcccaaggccgtgagagccatcatgcagaacagaagcatcgtgaactacgtgctgcacagactgcccagcgagcaggtgcacagatacgccagcgtgttccccgagaacttcgccgacctggagcaggagatcgacttcctgaccaagaagctgttcgagttcagcttcgaggagctgctgcacgagaaggacgtgatcctgaacaacagcagaagccacaagcccagcctggagatcgagagactgaaggccatcaccggcctgtacctgagcgtggcctacatcgccatcaagaacatcgtgaaggccaacgccagatactacatcgccttcgccgtgttcgagagagacaaggagctggtgaaggccaaggacgccagaatccagaccaagatccccgagacagacttccccgactacttctgcctgacccagtactacctggacagagacgaggagaagaagttccccggcgaccccagagacaaggaggccttcttcgagcacctgagaaagaccaagagacacttcagcaagcagtggagagagtggctgaacgagaagatcgccgacgccaagagcagccaggccaccggcctgctgctgagagaggccagaaacgacgtggagcacctgaacgtgctgagagccatccccgactacatccaggacttcagacacggcgagaagggcgagacagccatgaacagctacttcgagctgtaccactacctgatgcagagactgatgctgaagaacaccgagctggacctgagccactggagcggctggatcatgagaagcggcagacccgacagagacttgatccagatcgccttcgtgagcctggcctacaacctgcccagatacagaaacctgaccaaggagcaccacttcgacgacaccgtgctgcagaagatcagagagaaggagagcctggacacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagataactctacaaacctgtagggttctgagacataacatttccgaagacgacaagatttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 162 - DNA, 25 bp, synthetic construct
  source 1..25: mol_type = other DNA
ataacatttccgaagacgacaagat

SEQ ID NO: 163 - DNA, 31 bp, synthetic construct
  source 1..31: mol_type = other DNA
taccacggcgcagccctggagacacccaagc

SEQ ID NO: 164 - DNA, 32 bp, synthetic construct
  source 1..32: mol_type = other DNA
ggtgaatgcgtagaactcgtcggtgatctgct

SEQ ID NO: 165 - DNA, 33 bp, synthetic construct; identical to SEQ ID NO: 129
  source 1..33: mol_type = other DNA

SEQ ID NO: 166 - DNA, 27 bp, synthetic construct
  source 1..27: mol_type = other DNA
cagggctgcgccgtggtagccctcggt

SEQ ID NO: 167 - DNA, 31 bp, synthetic construct; identical to SEQ ID NO: 163
  source 1..31: mol_type = other DNA

SEQ ID NO: 168 - DNA, 33 bp, synthetic construct; identical to SEQ ID NO: 140
  source 1..33: mol_type = other DNA

SEQ ID NO: 169 - DNA, 31 bp, synthetic construct
  source 1..31: mol_type = other DNA
tcatcatcgacgcatacgccagcggcctgag

SEQ ID NO: 170 - DNA, 27 bp, synthetic construct
  source 1..27: mol_type = other DNA
cagggctgcgccgtggtagccctcggt

SEQ ID NO: 171 - DNA, 33 bp, synthetic construct; identical to SEQ ID NO: 129
  source 1..33: mol_type = other DNA

SEQ ID NO: 172 - DNA, 33 bp, synthetic construct; identical to SEQ ID NO: 140
  source 1..33: mol_type = other DNA

SEQ ID NO: 173 - DNA, 31 bp, synthetic construct; identical to SEQ ID NO: 169
  source 1..31: mol_type = other DNA

SEQ ID NO: 174 - DNA, 32 bp, synthetic construct; identical to SEQ ID NO: 164
  source 1..32: mol_type = other DNA

SEQ ID NO: 175 - DNA, 31 bp, synthetic construct; identical to SEQ ID NO: 163
  source 1..31: mol_type = other DNA

SEQ ID NO: 176 - DNA, 32 bp, synthetic construct; identical to SEQ ID NO: 164
  source 1..32: mol_type = other DNA

SEQ ID NO: 177 - DNA, 33 bp, synthetic construct; identical to SEQ ID NO: 129
  source 1..33: mol_type = other DNA

SEQ ID NO: 178 - DNA, 33 bp, synthetic construct; identical to SEQ ID NO: 140
  source 1..33: mol_type = other DNA

SEQ ID NO: 179 - DNA, 31 bp, synthetic construct; identical to SEQ ID NO: 169
  source 1..31: mol_type = other DNA

SEQ ID NO: 180 - DNA, 27 bp, synthetic construct
  source 1..27: mol_type = other DNA
cagggctgcgccgtggtagccctcggt

SEQ ID NO: 181 - DNA, 33 bp, synthetic construct; identical to SEQ ID NO: 119
  source 1..33: mol_type = other DNA

SEQ ID NO: 182 - DNA, 31 bp, synthetic construct
  source 1..31: mol_type = other DNA
gatctctgcgatcttcaccaggttcatgccc

SEQ ID NO: 183 - DNA, 35 bp, synthetic construct; identical to SEQ ID NO: 137
  source 1..35: mol_type = other DNA

SEQ ID NO: 184 - DNA, 27 bp, synthetic construct
  source 1..27: mol_type = other DNA
acggcctcgtcgattcttctcacctcg

SEQ ID NO: 185 - DNA, 33 bp, synthetic construct; identical to SEQ ID NO: 119
  source 1..33: mol_type = other DNA

SEQ ID NO: 186 - DNA, 35 bp, synthetic construct; identical to SEQ ID NO: 160
  source 1..35: mol_type = other DNA

SEQ ID NO: 187 - DNA, 32 bp, synthetic construct
  source 1..32: mol_type = other DNA
caaggccaagtacatcctgaagtacaaggacg

SEQ ID NO: 188 - DNA, 27 bp, synthetic construct
  source 1..27: mol_type = other DNA
acggcctcgtcgattcttctcacctcg

SEQ ID NO: 189 - DNA, 33 bp, synthetic construct; identical to SEQ ID NO: 119
  source 1..33: mol_type = other DNA

SEQ ID NO: 190 - DNA, 31 bp, synthetic construct
  source 1..31: mol_type = other DNA
tctcgctgggggccaccagcagggcctcgat

SEQ ID NO: 191 - DNA, 25 bp, synthetic construct
  source 1..25: mol_type = other DNA
tggtggcccccagcgagaaggtggg

SEQ ID NO: 192 - DNA, 27 bp, synthetic construct
  source 1..27: mol_type = other DNA
acggcctcgtcgattcttctcacctcg

SEQ ID NO: 193 - DNA, 34 bp, synthetic construct; identical to SEQ ID NO: 123
  source 1..34: mol_type = other DNA

SEQ ID NO: 194 - DNA, 31 bp, synthetic construct; identical to SEQ ID NO: 182
  source 1..31: mol_type = other DNA

SEQ ID NO: 195 - DNA, 35 bp, synthetic construct; identical to SEQ ID NO: 137
  source 1..35: mol_type = other DNA

SEQ ID NO: 196 - DNA, 25 bp, synthetic construct
  source 1..25: mol_type = other DNA
catggccacggcgttcttgctcagg

SEQ ID NO: 197 - DNA, 34 bp, synthetic construct; identical to SEQ ID NO: 123
  source 1..34: mol_type = other DNA

SEQ ID NO: 198 - DNA, 35 bp, synthetic construct; identical to SEQ ID NO: 160
  source 1..35: mol_type = other DNA

SEQ ID NO: 199 - DNA, 32 bp, synthetic construct; identical to SEQ ID NO: 187
  source 1..32: mol_type = other DNA

SEQ ID NO: 200 - DNA, 25 bp, synthetic construct
  source 1..25: mol_type = other DNA
catggccacggcgttcttgctcagg

SEQ ID NO: 201 - DNA, 25 bp, synthetic construct
  source 1..25: mol_type = other DNA
tggtggcccccagcgagaaggtggg

SEQ ID NO: 202 - DNA, 25 bp, synthetic construct
  source 1..25: mol_type = other DNA
catggccacggcgttcttgctcagg

SEQ ID NO: 203 - DNA, 34 bp, synthetic construct; identical to SEQ ID NO: 123
  source 1..34: mol_type = other DNA

SEQ ID NO: 204 - DNA, 31 bp, synthetic construct; identical to SEQ ID NO: 190
  source 1..31: mol_type = other DNA

SEQ ID NO: 205 - DNA, 33 bp, synthetic construct; identical to SEQ ID NO: 119
  source 1..33: mol_type = other DNA

SEQ ID NO: 206 - DNA, 25 bp, synthetic construct
  source 1..25: mol_type = other DNA
catggccacggcgttcttgctcagg

SEQ ID NO: 207 - DNA, 34 bp, synthetic construct; identical to SEQ ID NO: 123
  source 1..34: mol_type = other DNA

SEQ ID NO: 208 - DNA, 31 bp, synthetic construct; identical to SEQ ID NO: 182
  source 1..31: mol_type = other DNA

SEQ ID NO: 209 - DNA, 35 bp, synthetic construct; identical to SEQ ID NO: 137
  source 1..35: mol_type = other DNA

SEQ ID NO: 210 - DNA, 27 bp, synthetic construct
  source 1..27: mol_type = other DNA
acggcctcgtcgattcttctcacctcg

SEQ ID NO: 211 - DNA, 33 bp, synthetic construct; identical to SEQ ID NO: 119
  source 1..33: mol_type = other DNA

SEQ ID NO: 212 - DNA, 31 bp, synthetic construct; identical to SEQ ID NO: 190
  source 1..31: mol_type = other DNA

SEQ ID NO: 213 - DNA, 25 bp, synthetic construct
  source 1..25: mol_type = other DNA
tggtggcccccagcgagaaggtggg

SEQ ID NO: 214 - DNA, 25 bp, synthetic construct
  source 1..25: mol_type = other DNA
catggccacggcgttcttgctcagg

SEQ ID NO: 215 - DNA, 34 bp, synthetic construct; identical to SEQ ID NO: 123
  source 1..34: mol_type = other DNA

SEQ ID NO: 216 - DNA, 27 bp, synthetic construct
  source 1..27: mol_type = other DNA
acggcctcgtcgattcttctcacctcg

SEQ ID NO: 217 - DNA, 32 bp, synthetic construct
  source 1..32: mol_type = other DNA
acaagaaatccaaggccaagagaatgggcgtg

SEQ ID NO: 218 - DNA, 32 bp, synthetic construct
  source 1..32: mol_type = other DNA
ggccttggatttcttgtccttgctcatgtcga

SEQ ID NO: 219 - DNA, 34 bp, synthetic construct
  source 1..34: mol_type = other DNA
agaatgggcgtgaagtccctgctggcccacggcg

SEQ ID NO: 220 - DNA, 32 bp, synthetic construct
  source 1..32: mol_type = other DNA
gacttcacgcccattctcttggccttggtttt

SEQ ID NO: 221 - DNA, 32 bp, synthetic construct
  source 1..32: mol_type = other DNA
cttcctggaaaagaacgccgtgaacatggcca

SEQ ID NO: 222 - DNA, 34 bp, synthetic construct
  source 1..34: mol_type = other DNA
cgttcttttccaggaagtccttgttcaccttctt

SEQ ID NO: 223 - DNA, 33 bp, synthetic construct
  source 1..33: mol_type = other DNA
gcaagaacaaggtgaacatggccatcctgttcg

SEQ ID NO: 224 - DNA, 33 bp, synthetic construct
  source 1..33: mol_type = other DNA
gttcaccttgttcttgctcaggaagtccttgtt

SEQ ID NO: 225 - DNA, 32 bp, synthetic construct
  source 1..32: mol_type = other DNA
gtgaacatagccatcctgttcgacctgctgaa

SEQ ID NO: 226 - DNA, 32 bp, synthetic construct
  source 1..32: mol_type = other DNA
aggatggctatgttcacggcgttcttgctcag

SEQ ID NO: 227 - DNA, 37 bp, synthetic construct
  source 1..37: mol_type = other DNA
tgttcgacctgctgaaggccagagacgtggagcagaa

SEQ ID NO: 228 - DNA, 32 bp, synthetic construct
  source 1..32: mol_type = other DNA
cttcagcaggtcgaacaggatggccatgttca

SEQ ID NO: 229 - DNA, 35 bp, synthetic construct
  source 1..35: mol_type = other DNA
cgacgagtactacagattcaccatcagaaaggacg

SEQ ID NO: 230 - DNA, 32 bp, synthetic construct
  source 1..32: mol_type = other DNA
atctgtagtactcgtcggtgatctgcttcttc

SEQ ID NO: 231 - DNA, 37 bp, synthetic construct
  source 1..37: mol_type = other DNA
aacctgggcttcaacctggtgaagatcagagagatca

SEQ ID NO: 232 - DNA, 32 bp, synthetic construct
  source 1..32: mol_type = other DNA
aggttgaagcccaggttcttgccgtcctttct

SEQ ID NO: 233 - DNA, 32 bp, synthetic construct
  source 1..32: mol_type = other DNA
catgagcctggtgaagatcagagagatcatca

SEQ ID NO: 234 - DNA, 32 bp, synthetic construct
  source 1..32: mol_type = other DNA
tcttcaccaggctcatgcccaggttcttgccg

SEQ ID NO: 235 - DNA, 37 bp, synthetic construct
  source 1..37: mol_type = other DNA
gcatgaacatcgtgaagatcagagagatcatcatcga

SEQ ID NO: 236 - DNA, 32 bp, synthetic construct
  source 1..32: mol_type = other DNA
cttcacgatgttcatgcccaggttcttgccgt

SEQ ID NO: 237 - DNA, 40 bp, synthetic construct
  source 1..40: mol_type = other DNA
ggcatgaacctgaagaagatcagagagatcatcatcgaca

SEQ ID NO: 238 - DNA, 33 bp, synthetic construct
  source 1..33: mol_type = other DNA
cttcttcaggttcatgcccaggttcttgccgtc

SEQ ID NO: 239 - DNA, 32 bp, synthetic construct
  source 1..32: mol_type = other DNA
gagatcatgatcgacagatacgccagcggcct

SEQ ID NO: 240 - DNA, 34 bp, synthetic construct
  source 1..34: mol_type = other DNA
ctgtcgatcatgatctctctgatcttcaccaggt

SEQ ID NO: 241 - DNA, 32 bp, synthetic construct
  source 1..32: mol_type = other DNA
atcatcgagagatacgccagcggcctgagaga

SEQ ID NO: 242 - DNA, 34 bp, synthetic construct
  source 1..34: mol_type = other DNA
gcgtatctctcgatgatgatctctctgatcttca

SEQ ID NO: 243 - DNA, 32 bp, synthetic construct
  source 1..32: mol_type = other DNA
acaagaagtacgacccccacagacagaagatc

SEQ ID NO: 244 - DNA, 32 bp, synthetic construct
  source 1..32: mol_type = other DNA
ggggtcgtacttcttgtctctcaggccgctgg

SEQ ID NO: 245 - DNA, 36 bp, synthetic construct
  source 1..36: mol_type = other DNA
aagaagcacgactcccacagacagaagatcaacgtg

SEQ ID NO: 246 - DNA, 32 bp, synthetic construct
  source 1..32: mol_type = other DNA
tgggagtcgtgcttcttgtctctcaggccgct

SEQ ID NO: 247 - DNA, 33 bp, synthetic construct
  source 1..33: mol_type = other DNA
ccgtcagacagaagatcaacgtgatcgccgact

SEQ ID NO: 248 - DNA, 37 bp, synthetic construct
  source 1..37: mol_type = other DNA
tgatcttctgtctgacggggtcgtgcttcttgtctct

SEQ ID NO: 249 - DNA, 32 bp, synthetic construct
  source 1..32: mol_type = other DNA
gaagatctacgtgatcgccgacttcctgatct

SEQ ID NO: 250 - DNA, 32 bp, synthetic construct
  source 1..32: mol_type = other DNA
cgatcacgtagatcttctgtctgtgggggtcg

SEQ ID NO: 251 - DNA, 32 bp, synthetic construct
  source 1..32: mol_type = other DNA
caggaccagggcatcatcgacaagaccgtgag

SEQ ID NO: 252 - DNA, 34 bp, synthetic construct
  source 1..34: mol_type = other DNA
atgatgccctggtcctggctcagggctctgaaga

SEQ ID NO: 253 - DNA, 34 bp, synthetic construct
  source 1..34: mol_type = other DNA
tcatcgacaagctcgtgagcagcctgagactgac

SEQ ID NO: 254 - DNA, 33 bp, synthetic construct
  source 1..33: mol_type = other DNA
tcacgagcttgtcgatgatgccctggttctggc

SEQ ID NO: 255 - DNA, 32 bp, synthetic construct
  source 1..32: mol_type = other DNA
tgagactgtccaaggacgaggaggagaaggac

SEQ ID NO: 256 - DNA, 32 bp, synthetic construct
  source 1..32: mol_type = other DNA
gtccttggacagtctcaggctgctcacggtct

SEQ ID NO: 257 - DNA, 37 bp, synthetic construct
  source 1..37: mol_type = other DNA
aaggacgaggaggacaaggaccacgtgtaccagaacg

SEQ ID NO: 258 - DNA, 32 bp, synthetic construct
  source 1..32: mol_type = other DNA
ttgtcctcctcgtccttggtcagtctcaggct

SEQ ID NO: 259 - DNA, 35 bp, synthetic construct
  source 1..35: mol_type = other DNA
acgtgtaccagaacgaggccgagctggtgtggggc

SEQ ID NO: 260 - DNA, 33 bp, synthetic construct
  source 1..33: mol_type = other DNA
cctcgttctggtacacgtggtccttctcctcct

SEQ ID NO: 261 - DNA, 32 bp, synthetic construct
  source 1..32: mol_type = other DNA
aaggtgagcaactgcctgaccccctacttcaa

SEQ ID NO: 262 - DNA, 34 bp, synthetic construct
  source 1..34: mol_type = other DNA
aggcagttgctcaccttgccccacaccagctcgg

SEQ ID NO: 263 - DNA, 32 bp, synthetic construct
  source 1..32: mol_type = other DNA
atggtgaggaactgcctgaccccctacttcaa

SEQ ID NO: 264 - DNA, 32 bp, synthetic construct
  source 1..32: mol_type = other DNA
aggcagttcctcaccatgccccacaccagctc

SEQ ID NO: 265 - DNA, 33 bp, synthetic construct
  source 1..33: mol_type = other DNA
caagaacgggtacatcctgaagtacaaggacgc

SEQ ID NO: 266 - DNA, 34 bp, synthetic construct
  source 1..34: mol_type = other DNA
aggatgtacccgttcttggggtcgttgaagtagg

SEQ ID NO: 267 - DNA, 32 bp, synthetic construct
  source 1..32: mol_type = other DNA
agaacaaggacatcctgaagtacaaggacgcc

SEQ ID NO: 268 - DNA, 32 bp, synthetic construct
  source 1..32: mol_type = other DNA
caggatgtccttgttcttggggtcgttgaagt

SEQ ID NO: 269 - DNA, 33 bp, synthetic construct
  source 1..33: mol_type = other DNA
agtacaagctcgccaagacccccggcgacttcg

SEQ ID NO: 270 - DNA, 38 bp, synthetic construct
  source 1..38: mol_type = other DNA
tcttggcgagcttgtacttcaggatgtacttgttcttg

SEQ ID NO: 271 - DNA, 32 bp, synthetic construct
  source 1..32: mol_type = other DNA
ttcgaggagtggatcaccagcaagatcagcga

SEQ ID NO: 272 - DNA, 32 bp, synthetic construct
  source 1..32: mol_type = other DNA
gtgatccactcctcgaagtcgccgggggtctt

SEQ ID NO: 273 - DNA, 33 bp, synthetic construct
  source 1..33: mol_type = other DNA
caagatcaaggaggacgacggcgagcccttcgt

SEQ ID NO: 274 - DNA, 33 bp, synthetic construct
  source 1..33: mol_type = other DNA
tcgtcctccttgatcttgctggtgatccagtcc

SEQ ID NO: 275 - DNA, 19 bp, synthetic construct
  source 1..19: mol_type = other DNA
ccagggaccatgttttgcc

SEQ ID NO: 276 - DNA, 21 bp, synthetic construct
  source 1..21: mol_type = other DNA
cgaagacgacaagatggacaa

SEQ ID NO: 277 - DNA, 36 bp, synthetic construct
  source 1..36: mol_type = other DNA
  variation 26: note = n is a or g
ggaagatnactctacaaacctgtagngnnnnnnnnn

SEQ ID NO: 278 - DNA, 36 bp, synthetic construct; identical to SEQ ID NO: 277
  source 1..36: mol_type = other DNA
  variation 8: note = n is a or g
  variation 26: note = n is a or g
  variation 28: note = n is a or t
  variation 29: note = n is c or t
  variation 30: note = n is c or t
  variation 31: note = n is g or t
  variation 32: note = n is g or t
  variation 33: note = n is a or g
  variation 34: note = n is c or g
  variation 35: note = n is a or g
  variation 36: note = n is c or g

SEQ ID NO: 279 - protein, 17 aa, synthetic construct
AEAAAKEAAAKEAAAKA

SEQ ID NO: 280 - protein, 15 aa, synthetic construct
GGGGSGGGGSGGGGS

SEQ ID NO: 281 - DNA, 36 bp, synthetic construct
  source 1..36: mol_type = other DNA
ggaagataactctacaaacctgtagggttctgagac

SEQ ID NO: 282 - RNA, 36 nt, synthetic construct
  source 1..36: mol_type = other RNA
ggaagataactctacaaacctgtagagttctgagac

SEQ ID NO: 283 - protein, 25 aa, synthetic construct
LGHKLEYNYNSHNVYIMADKQKNGI

SEQ ID NO: 284 - DNA, 73 bp, synthetic construct
  source 1..73: mol_type = other DNA
tggggcacaagctggagtacaactacaacagccacaacgtctatatcatggccgacaagcagaagaacggcat